SEQUENCE LISTING (209 sequences)

SEQ ID NO: 1 - protein, 117 aa, synthetic construct
  REGION 1..117: note = Description of Artificial Sequence: Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, synthetic construct
  REGION 1..107: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 9 aa, synthetic construct
  REGION 1..9: note = Description of Artificial Sequence: Synthetic peptide
GSFSGYYWS

SEQ ID NO: 4 - protein, 16 aa, synthetic construct
  REGION 1..16: note = Description of Artificial Sequence: Synthetic peptide
EIDHSGSTNYNPSLKS

SEQ ID NO: 5 - protein, 11 aa, synthetic construct
  REGION 1..11: note = Description of Artificial Sequence: Synthetic peptide
ARARGPWSFDP

SEQ ID NO: 6 - protein, 117 aa, synthetic construct; identical to SEQ ID NO: 1
  REGION 1..117: note = Description of Artificial Sequence: Synthetic polypeptide

SEQ ID NO: 7 - protein, 108 aa, synthetic construct
  REGION 1..108: note = Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 8 - protein, 117 aa, synthetic construct; identical to SEQ ID NO: 1
  REGION 1..117: note = Description of Artificial Sequence: Synthetic polypeptide

SEQ ID NO: 9 - protein, 106 aa, synthetic construct
  REGION 1..106: note = Description of Artificial Sequence: Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 10 - protein, 117 aa, synthetic construct; identical to SEQ ID NO: 1
  REGION 1..117: note = Description of Artificial Sequence: Synthetic polypeptide

SEQ ID NO: 11 - protein, 106 aa, synthetic construct
  REGION 1..106: note = Description of Artificial Sequence: Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 12 - protein, 117 aa, synthetic construct; identical to SEQ ID NO: 1
  REGION 1..117: note = Description of Artificial Sequence: Synthetic polypeptide

SEQ ID NO: 13 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 14 - protein, 117 aa, synthetic construct
  REGION 1..117: note = Description of Artificial Sequence: Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 15 - protein, 107 aa, synthetic construct
  REGION 1..107: note = Description of Artificial Sequence: Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 16 - protein, 117 aa, synthetic construct; identical to SEQ ID NO: 1
  REGION 1..117: note = Description of Artificial Sequence: Synthetic polypeptide

SEQ ID NO: 17 - protein, 107 aa, synthetic construct
  REGION 1..107: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 18 - protein, 117 aa, synthetic construct; identical to SEQ ID NO: 1
  REGION 1..117: note = Description of Artificial Sequence: Synthetic polypeptide

SEQ ID NO: 19 - protein, 107 aa, synthetic construct
  REGION 1..107: note = Description of Artificial Sequence: Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 20 - protein, 117 aa, synthetic construct; identical to SEQ ID NO: 1
  REGION 1..117: note = Description of Artificial Sequence: Synthetic polypeptide

SEQ ID NO: 21 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 22 - protein, 117 aa, synthetic construct; identical to SEQ ID NO: 1
  REGION 1..117: note = Description of Artificial Sequence: Synthetic polypeptide

SEQ ID NO: 23 - protein, 106 aa, synthetic construct
  REGION 1..106: note = Description of Artificial Sequence: Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 24 - protein, 117 aa, synthetic construct; identical to SEQ ID NO: 1
  REGION 1..117: note = Description of Artificial Sequence: Synthetic polypeptide

SEQ ID NO: 25 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 26 - protein, 117 aa, synthetic construct; identical to SEQ ID NO: 1
  REGION 1..117: note = Description of Artificial Sequence: Synthetic polypeptide

SEQ ID NO: 27 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 28 - protein, 117 aa, synthetic construct; identical to SEQ ID NO: 1
  REGION 1..117: note = Description of Artificial Sequence: Synthetic polypeptide

SEQ ID NO: 29 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 30 - protein, 117 aa, synthetic construct; identical to SEQ ID NO: 1
  REGION 1..117: note = Description of Artificial Sequence: Synthetic polypeptide

SEQ ID NO: 31 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 32 - protein, 117 aa, synthetic construct; identical to SEQ ID NO: 1
  REGION 1..117: note = Description of Artificial Sequence: Synthetic polypeptide

SEQ ID NO: 33 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 34 - protein, 117 aa, synthetic construct; identical to SEQ ID NO: 1
  REGION 1..117: note = Description of Artificial Sequence: Synthetic polypeptide

SEQ ID NO: 35 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 36 - protein, 117 aa, synthetic construct; identical to SEQ ID NO: 1
  REGION 1..117: note = Description of Artificial Sequence: Synthetic polypeptide

SEQ ID NO: 37 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 38 - protein, 117 aa, synthetic construct; identical to SEQ ID NO: 1
  REGION 1..117: note = Description of Artificial Sequence: Synthetic polypeptide

SEQ ID NO: 39 - protein, 106 aa, synthetic construct
  REGION 1..106: note = Description of Artificial Sequence: Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 40 - protein, 117 aa, synthetic construct; identical to SEQ ID NO: 1
  REGION 1..117: note = Description of Artificial Sequence: Synthetic polypeptide

SEQ ID NO: 41 - protein, 107 aa, synthetic construct
  REGION 1..107: note = Description of Artificial Sequence: Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 42 - protein, 117 aa, synthetic construct; identical to SEQ ID NO: 1
  REGION 1..117: note = Description of Artificial Sequence: Synthetic polypeptide

SEQ ID NO: 43 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 44 - protein, 125 aa, synthetic construct
  REGION 1..125: note = Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 45 - protein, 9 aa, synthetic construct
  REGION 1..9: note = Description of Artificial Sequence: Synthetic peptide
GTFSSYAIS

SEQ ID NO: 46 - protein, 17 aa, synthetic construct
  REGION 1..17: note = Description of Artificial Sequence: Synthetic peptide
GIIPIFGTANYAQKFQG

SEQ ID NO: 47 - protein, 18 aa, synthetic construct
  REGION 1..18: note = Description of Artificial Sequence: Synthetic peptide
ARGDSSIRHAYYYYGMDV

SEQ ID NO: 48 - protein, 113 aa, synthetic construct
  REGION 1..113: note = Description of Artificial Sequence: Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 49 - protein, 17 aa, synthetic construct
  REGION 1..17: note = Description of Artificial Sequence: Synthetic peptide
KSSQSVLYSSNNKNYLA

SEQ ID NO: 50 - protein, 7 aa, synthetic construct
  REGION 1..7: note = Description of Artificial Sequence: Synthetic peptide
WASTRES

SEQ ID NO: 51 - protein, 9 aa, synthetic construct
  REGION 1..9: note = Description of Artificial Sequence: Synthetic peptide
QQYYSTPIT

SEQ ID NO: 52 - protein, 121 aa, synthetic construct
  REGION 1..121: note = Description of Artificial Sequence: Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 53 - protein, 11 aa, synthetic construct
  REGION 1..11: note = Description of Artificial Sequence: Synthetic peptide
GSISSSSYYWG

SEQ ID NO: 54 - protein, 16 aa, synthetic construct
  REGION 1..16: note = Description of Artificial Sequence: Synthetic peptide
SIYYSGSTYYNPSLKS

SEQ ID NO: 55 - protein, 13 aa, synthetic construct
  REGION 1..13: note = Description of Artificial Sequence: Synthetic peptide
ARGSDRFHPYFDY

SEQ ID NO: 56 - protein, 107 aa, synthetic construct
  REGION 1..107: note = Description of Artificial Sequence: Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 57 - protein, 11 aa, synthetic construct
  REGION 1..11: note = Description of Artificial Sequence: Synthetic peptide
RASQSVSRYLA

SEQ ID NO: 58 - protein, 7 aa, synthetic construct
  REGION 1..7: note = Description of Artificial Sequence: Synthetic peptide
DASNRAT

SEQ ID NO: 59 - protein, 9 aa, synthetic construct
  REGION 1..9: note = Description of Artificial Sequence: Synthetic peptide
QQFDTWPPT

SEQ ID NO: 60 - protein, 117 aa, synthetic construct; identical to SEQ ID NO: 1
  REGION 1..117: note = Description of Artificial Sequence: Synthetic polypeptide

SEQ ID NO: 61 - protein, 106 aa, synthetic construct
  REGION 1..106: note = Description of Artificial Sequence: Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 62 - protein, 126 aa, synthetic construct
  REGION 1..126: note = Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 63 - protein, 9 aa, synthetic construct
  REGION 1..9: note = Description of Artificial Sequence: Synthetic peptide
GTFSSYAIS

SEQ ID NO: 64 - protein, 17 aa, synthetic construct
  REGION 1..17: note = Description of Artificial Sequence: Synthetic peptide
GIIPIFGTANYAQKFQG

SEQ ID NO: 65 - protein, 19 aa, synthetic construct
  REGION 1..19: note = Description of Artificial Sequence: Synthetic peptide
ARRGRKASGSFYYYYGMDV

SEQ ID NO: 66 - protein, 113 aa, synthetic construct [sequence omitted]

SEQ ID NO: 67 - protein, 17 aa, synthetic construct
  REGION 1..17: note = Description of Artificial Sequence: Synthetic peptide
ESSQSLLNSGNQKNYLT

SEQ ID NO: 68 - protein, 7 aa, synthetic construct
  REGION 1..7: note = Description of Artificial Sequence: Synthetic peptide
WASTRES

SEQ ID NO: 69 - protein, 9 aa, synthetic construct
  REGION 1..9: note = Description of Artificial Sequence: Synthetic peptide
QNDYSYPYT

SEQ ID NO: 70 - protein, 126 aa, synthetic construct
  REGION 1..126: note = Description of Artificial Sequence: Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 71 - protein, 9 aa, synthetic construct
  REGION 1..9: note = Description of Artificial Sequence: Synthetic peptide
YTFTSYYMH

SEQ ID NO: 72 - protein, 17 aa, synthetic construct
  REGION 1..17: note = Description of Artificial Sequence: Synthetic peptide
IINPSGGSTSYAQKFQG

SEQ ID NO: 73 - protein, 19 aa, synthetic construct
  REGION 1..19: note = Description of Artificial Sequence: Synthetic peptide
ARGAPNYGDTTHDYYYMDV

SEQ ID NO: 74 - protein, 107 aa, synthetic construct
  REGION 1..107: note = Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 75 - protein, 11 aa, synthetic construct
  REGION 1..11: note = Description of Artificial Sequence: Synthetic peptide
RASQSVSSNLA

SEQ ID NO: 76 - protein, 7 aa, synthetic construct
  REGION 1..7: note = Description of Artificial Sequence: Synthetic peptide
GASTRAT

SEQ ID NO: 77 - protein, 9 aa, synthetic construct
  REGION 1..9: note = Description of Artificial Sequence: Synthetic peptide
QQYDDWPFT

SEQ ID NO: 78 - protein, 124 aa, synthetic construct
  REGION 1..124: note = Description of Artificial Sequence: Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 79 - protein, 9 aa, synthetic construct
  REGION 1..9: note = Description of Artificial Sequence: Synthetic peptide
YTFTGYYMH

SEQ ID NO: 80 - protein, 17 aa, synthetic construct
  REGION 1..17: note = Description of Artificial Sequence: Synthetic peptide
WINPNSGGTNYAQKFQG

SEQ ID NO: 81 - protein, 17 aa, synthetic construct
  REGION 1..17: note = Description of Artificial Sequence: Synthetic peptide
ARDTGEYYDTDDHGMDV

SEQ ID NO: 82 - protein, 107 aa, synthetic construct
  REGION 1..107: note = Description of Artificial Sequence: Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 83 - protein, 50 aa, synthetic construct
  REGION 1..50: note = Description of Artificial Sequence: Synthetic polypeptide
GGSGGGGSGGGGSGGGGSGGGGSGGGGSGGGGSGGGGSGGGGSGGGGSGG

SEQ ID NO: 84 - protein, 50 aa, synthetic construct
  REGION 1..50: note = Description of Artificial Sequence: Synthetic polypeptide
GGGGSGGGGSGGGGSGGGGSGGGGSGGGGSGGGGSGGGGSGGGGSGGGGS

SEQ ID NO: 85 - protein, 9 aa, synthetic construct
  REGION 1..9: note = Description of Artificial Sequence: Synthetic peptide
QQDDYWPPT

SEQ ID NO: 86 - protein, 121 aa, synthetic construct
  REGION 1..121: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 87 - protein, 9 aa, synthetic construct
  REGION 1..9: note = Description of Artificial Sequence: Synthetic peptide
FTFSSYAMS

SEQ ID NO: 88 - protein, 17 aa, synthetic construct
  REGION 1..17: note = Description of Artificial Sequence: Synthetic peptide
AISGSGGSTYYADSVKG

SEQ ID NO: 89 - protein, 14 aa, synthetic construct
  REGION 1..14: note = Description of Artificial Sequence: Synthetic peptide
AKDGGYYDSGAGDY

SEQ ID NO: 90 - protein, 107 aa, synthetic construct
  REGION 1..107: note = Description of Artificial Sequence: Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 91 - protein, 11 aa, synthetic construct
  REGION 1..11: note = Description of Artificial Sequence: Synthetic peptide
RASQGIDSWLA

SEQ ID NO: 92 - protein, 7 aa, synthetic construct
  REGION 1..7: note = Description of Artificial Sequence: Synthetic peptide
AASSLQS

SEQ ID NO: 93 - protein, 9 aa, synthetic construct
  REGION 1..9: note = Description of Artificial Sequence: Synthetic peptide
QQGVSYPRT

SEQ ID NO: 94 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 95 - protein, 9 aa, synthetic construct
  REGION 1..9: note = Description of Artificial Sequence: Synthetic peptide
FTFSSYSMN

SEQ ID NO: 96 - protein, 17 aa, synthetic construct
  REGION 1..17: note = Description of Artificial Sequence: Synthetic peptide
SISSSSSYIYYADSVKG

SEQ ID NO: 97 - protein, 15 aa, synthetic construct
  REGION 1..15: note = Description of Artificial Sequence: Synthetic peptide
ARGAPMGAAAGWFDP

SEQ ID NO: 98 - protein, 107 aa, synthetic construct
  REGION 1..107: note = Description of Artificial Sequence: Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 99 - protein, 11 aa, synthetic construct
  REGION 1..11: note = Description of Artificial Sequence: Synthetic peptide
RASQGISSWLA

SEQ ID NO: 100 - protein, 7 aa, synthetic construct
  REGION 1..7: note = Description of Artificial Sequence: Synthetic peptide
AASSLQS

SEQ ID NO: 101 - protein, 9 aa, synthetic construct
  REGION 1..9: note = Description of Artificial Sequence: Synthetic peptide
QQGVSFPRT

SEQ ID NO: 102 - protein, 125 aa, synthetic construct
  REGION 1..125: note = Description of Artificial Sequence: Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 103 - protein, 40 aa, synthetic construct
  REGION 1..40: note = Description of Artificial Sequence: Synthetic polypeptide
GGGSGGGSGGGSGGGSGGGSGGGSGGGSGGGSGGGSGGGS

SEQ ID NO: 104 - protein, 40 aa, synthetic construct
  REGION 1..40: note = Description of Artificial Sequence: Synthetic polypeptide
GGSGGGSGGGSGGGSGGGSGGGSGGGSGGGSGGGSGGGSG

SEQ ID NO: 105 - protein, 18 aa, synthetic construct
  REGION 1..18: note = Description of Artificial Sequence: Synthetic peptide
AREGAGFAYGMDYYYMDV

SEQ ID NO: 106 - protein, 107 aa, synthetic construct
  REGION 1..107: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 107 - protein, 11 aa, synthetic construct
  REGION 1..11: note = Description of Artificial Sequence: Synthetic peptide
RASQSVSSYLA

SEQ ID NO: 108 - protein, 7 aa, synthetic construct
  REGION 1..7: note = Description of Artificial Sequence: Synthetic peptide
DASNRAT

SEQ ID NO: 109 - protein, 9 aa, synthetic construct
  REGION 1..9: note = Description of Artificial Sequence: Synthetic peptide
QQSDNWPFT

SEQ ID NO: 110 - protein, 121 aa, Homo sapiens
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 111 - protein, 110 aa, Homo sapiens
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 112 - protein, 115 aa, Homo sapiens
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 113 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 114 - protein, 120 aa, synthetic construct [sequence omitted]

SEQ ID NO: 115 - protein, 7 aa, synthetic construct
  REGION 1..7: note = Description of Artificial Sequence: Synthetic peptide
GFNIKDT

SEQ ID NO: 116 - protein, 6 aa, synthetic construct
  REGION 1..6: note = Description of Artificial Sequence: Synthetic peptide
YPTNGY

SEQ ID NO: 117 - protein, 11 aa, synthetic construct
  REGION 1..11: note = Description of Artificial Sequence: Synthetic peptide
WGGDGFYAMDY

SEQ ID NO: 118 - protein, 107 aa, synthetic construct [sequence omitted]

SEQ ID NO: 119 - protein, 8 aa, synthetic construct
  REGION 1..8: note = Description of Artificial Sequence: Synthetic peptide
QDVNTAVA

SEQ ID NO: 120 - protein, 7 aa, synthetic construct
  REGION 1..7: note = Description of Artificial Sequence: Synthetic peptide
SASFLYS

SEQ ID NO: 121 - protein, 9 aa, synthetic construct
  REGION 1..9: note = Description of Artificial Sequence: Synthetic peptide
QQHYTTPPT

SEQ ID NO: 122 - protein, 120 aa, synthetic construct
  REGION 1..120: note = Description of Artificial Sequence: Synthetic polypeptide
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSA

SEQ ID NO: 123 - protein, 7 aa, synthetic construct
  REGION 1..7: note = Description of Artificial Sequence: Synthetic peptide
GFTFTDY

SEQ ID NO: 124 - protein, 6 aa, synthetic construct
  REGION 1..6: note = Description of Artificial Sequence: Synthetic peptide
NPNSGG

SEQ ID NO: 125 - protein, 10 aa, synthetic construct
  REGION 1..10: note = Description of Artificial Sequence: Synthetic peptide
NLGPSFYFDY

SEQ ID NO: 126 - protein, 108 aa, synthetic construct
  REGION 1..108: note = Description of Artificial Sequence: Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 127 - protein, 8 aa, synthetic construct
  REGION 1..8: note = Description of Artificial Sequence: Synthetic peptide
QDVSIGVA

SEQ ID NO: 128 - protein, 7 aa, synthetic construct
  REGION 1..7: note = Description of Artificial Sequence: Synthetic peptide
SASYRYT

SEQ ID NO: 129 - protein, 9 aa, synthetic construct
  REGION 1..9: note = Description of Artificial Sequence: Synthetic peptide
QQYYIYPYT

SEQ ID NO: 130 - protein, 120 aa, synthetic construct
  REGION 1..120: note = Description of Artificial Sequence: Synthetic polypeptide
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 131 - protein, 7 aa, synthetic construct
  REGION 1..7: note = Description of Artificial Sequence: Synthetic peptide
GFNIKDT

SEQ ID NO: 132 - protein, 6 aa, synthetic construct
  REGION 1..6: note = Description of Artificial Sequence: Synthetic peptide
YPTNGY

SEQ ID NO: 133 - protein, 11 aa, synthetic construct
  REGION 1..11: note = Description of Artificial Sequence: Synthetic peptide
WGGDGFYAMDY

SEQ ID NO: 134 - protein, 107 aa, synthetic construct
  REGION 1..107: note = Description of Artificial Sequence: Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 135 - protein, 8 aa, synthetic construct
  REGION 1..8: note = Description of Artificial Sequence: Synthetic peptide
QDVNTAVA

SEQ ID NO: 136 - protein, 7 aa, synthetic construct
  REGION 1..7: note = Description of Artificial Sequence: Synthetic peptide
SASFRYT

SEQ ID NO: 137 - protein, 9 aa, synthetic construct
  REGION 1..9: note = Description of Artificial Sequence: Synthetic peptide
QQHYTTPPT

SEQ ID NO: 138 - protein, 1255 aa, Homo sapiens [sequence omitted]

SEQ ID NO: 139 - protein, 247 aa, synthetic construct
  REGION 1..247: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 140 - protein, 475 aa, synthetic construct
  REGION 1..475: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 141 - protein, 451 aa, synthetic construct
  REGION 1..451: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 142 - protein, 210 aa, synthetic construct
  REGION 1..210: note = Description of Artificial Sequence: Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 20 aa, synthetic construct
  REGION 1..20: note = Description of Artificial Sequence: Synthetic peptide
GGGGSGGGGSGGGGSGGGGS

SEQ ID NO: 144 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 145 - protein, 451 aa, synthetic construct
  REGION 1..451: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 146 - protein, 475 aa, synthetic construct
  REGION 1..475: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 147 - protein, 451 aa, synthetic construct
  REGION 1..451: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 450 aa, synthetic construct
  REGION 1..450: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 214 aa, synthetic construct
  REGION 1..214: note = Description of Artificial Sequence: Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 15 aa, synthetic construct
  REGION 1..15: note = Description of Artificial Sequence: Synthetic peptide
GGGGSGGGGSGGGGS

SEQ ID NO: 151 - protein, 450 aa, synthetic construct
  REGION 1..450: note = Description of Artificial Sequence: Synthetic polypeptide
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 152 - protein, 100 aa, synthetic construct
  REGION 1..100: note = Description of Artificial Sequence: Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 153 - protein, 214 aa, synthetic construct
  REGION 1..214: note = Description of Artificial Sequence: Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 100 aa, synthetic construct
  REGION 1..100: note = Description of Artificial Sequence: Synthetic polypeptide
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 155 - protein, 450 aa, synthetic construct
  REGION 1..450: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 156 - protein, 475 aa, synthetic construct
  REGION 1..475: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 157 - protein, 451 aa, synthetic construct
  REGION 1..451: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 158 - protein, 5 aa, synthetic construct
  REGION 1..5: note = Description of Artificial Sequence: Synthetic peptide
SYAIS

SEQ ID NO: 159 - protein, 16 aa, synthetic construct
  REGION 1..16: note = Description of Artificial Sequence: Synthetic peptide
GDSSIRHAYYYYGMDV

SEQ ID NO: 160 - protein, 7 aa, synthetic construct
  REGION 1..7: note = Description of Artificial Sequence: Synthetic peptide
SSSYYWG

SEQ ID NO: 161 - protein, 11 aa, synthetic construct
  REGION 1..11: note = Description of Artificial Sequence: Synthetic peptide
GSDRFHPYFDY

SEQ ID NO: 162 - protein, 5 aa, synthetic construct
  REGION 1..5: note = Description of Artificial Sequence: Synthetic peptide
SYYMH

SEQ ID NO: 163 - protein, 17 aa, synthetic construct
  REGION 1..17: note = Description of Artificial Sequence: Synthetic peptide
GAPNYGDTTHDYYYMDV

SEQ ID NO: 164 - protein, 5 aa, synthetic construct
  REGION 1..5: note = Description of Artificial Sequence: Synthetic peptide
GYYMH

SEQ ID NO: 165 - protein, 15 aa, synthetic construct
  REGION 1..15: note = Description of Artificial Sequence: Synthetic peptide
DTGEYYDTDDHGMDV

SEQ ID NO: 166 - protein, 5 aa, synthetic construct
  REGION 1..5: note = Description of Artificial Sequence: Synthetic peptide
SYAMS

SEQ ID NO: 167 - protein, 12 aa, synthetic construct
  REGION 1..12: note = Description of Artificial Sequence: Synthetic peptide
DGGYYDSGAGDY

SEQ ID NO: 168 - protein, 5 aa, synthetic construct
  REGION 1..5: note = Description of Artificial Sequence: Synthetic peptide
SYSMN

SEQ ID NO: 169 - protein, 13 aa, synthetic construct
  REGION 1..13: note = Description of Artificial Sequence: Synthetic peptide
GAPMGAAAGWFDP

SEQ ID NO: 170 - protein, 16 aa, synthetic construct
  REGION 1..16: note = Description of Artificial Sequence: Synthetic peptide
EGAGFAYGMDYYYMDV

SEQ ID NO: 171 - protein, 249 aa, synthetic construct
  REGION 1..249: note = Description of Artificial Sequence: Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSA

SEQ ID NO: 172 - protein, 15 aa, synthetic construct
  REGION 1..15: note = Description of Artificial Sequence: Synthetic peptide
ARGAPIGAAAGWFDP

SEQ ID NO: 173 - protein, 13 aa, synthetic construct
  REGION 1..13: note = Description of Artificial Sequence: Synthetic peptide
GAPIGAAAGWFDP

SEQ ID NO: 174 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 175 - protein, 15 aa, synthetic construct
  REGION 1..15: note = Description of Artificial Sequence: Synthetic peptide
ARGAPQGAAAGWFDP

SEQ ID NO: 176 - protein, 13 aa, synthetic construct
  REGION 1..13: note = Description of Artificial Sequence: Synthetic peptide
GAPQGAAAGWFDP

SEQ ID NO: 177 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 178 - protein, 15 aa, synthetic construct
  REGION 1..15: note = Description of Artificial Sequence: Synthetic peptide
ARGAPLGAAAGWFDP

SEQ ID NO: 179 - protein, 13 aa, synthetic construct
  REGION 1..13: note = Description of Artificial Sequence: Synthetic peptide
GAPLGAAAGWFDP

SEQ ID NO: 180 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 181 - protein, 15 aa, synthetic construct
  REGION 1..15: note = Description of Artificial Sequence: Synthetic peptide
ARGAPFGAAAGWFDP

SEQ ID NO: 182 - protein, 13 aa, synthetic construct
  REGION 1..13: note = Description of Artificial Sequence: Synthetic peptide
GAPFGAAAGWFDP

SEQ ID NO: 183 - protein, 122 aa, synthetic construct
  REGION 1..122: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 184 - protein, 15 aa, synthetic construct
  REGION 1..15: note = Description of Artificial Sequence: Synthetic peptide
ARGAPVGAAAGWFDP

SEQ ID NO: 185 - protein, 13 aa, synthetic construct
  REGION 1..13: note = Description of Artificial Sequence: Synthetic peptide
GAPVGAAAGWFDP

SEQ ID NO: 186 - protein, 122 aa, synthetic construct
  REGION 1..122: note = Description of Artificial Sequence: Synthetic polypeptide
  MOD_RES 102: note = M, L, I, V, Q, or F
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 187 - protein, 15 aa, synthetic construct
  REGION 1..15: note = Description of Artificial Sequence: Synthetic peptide
  MOD_RES 6: note = M, L, I, V, Q, or F
ARGAPXGAAAGWFDP

SEQ ID NO: 188 - protein, 13 aa, synthetic construct
  REGION 1..13: note = Description of Artificial Sequence: Synthetic peptide
  MOD_RES 4: note = M, L, I, V, Q, or F
GAPXGAAAGWFDP

SEQ ID NO: 189 - protein, 248 aa, synthetic construct
  REGION 1..248: note = Description of Artificial Sequence: Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSA

SEQ ID NO: 190 - protein, 476 aa, synthetic construct
  REGION 1..476: note = Description of Artificial Sequence: Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 191 - protein, 476 aa, synthetic construct
  REGION 1..476: note = Description of Artificial Sequence: Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 192 - protein, 477 aa, synthetic construct
  REGION 1..477: note = Description of Artificial Sequence: Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 193 - protein, 477 aa, synthetic construct
  REGION 1..477: note = Description of Artificial Sequence: Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 194 - protein, 451 aa, synthetic construct
  REGION 1..451: note = Description of Artificial Sequence: Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 195 - protein, 120 aa, synthetic construct
  REGION 1..120: note = Description of Artificial Sequence: Synthetic polypeptide
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 196 - protein, 107 aa, synthetic construct
  REGION 1..107: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIK

SEQ ID NO: 197 - protein, 120 aa, synthetic construct
  REGION 1..120: note = Description of Artificial Sequence: Synthetic polypeptide
EVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSA

SEQ ID NO: 198 - protein, 108 aa, synthetic construct
  REGION 1..108: note = Description of Artificial Sequence: Synthetic polypeptide
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKR

SEQ ID NO: 199 - protein, 121 aa, synthetic construct
  REGION 1..121: note = Description of Artificial Sequence: Synthetic polypeptide
QVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSA

SEQ ID NO: 200 - protein, 108 aa, synthetic construct
  REGION 1..108: note = Description of Artificial Sequence: Synthetic polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKR

SEQ ID NO: 201 - protein, 20 aa, synthetic construct
  REGION 1..20: note = Description of Artificial Sequence: Synthetic peptide
GSGSGSGSGSGSGSGSGSGS

SEQ ID NO: 202 - protein, 30 aa, synthetic construct
  REGION 1..30: note = Description of Artificial Sequence: Synthetic polypeptide
GGSGGSGGSGGSGGSGGSGGSGGSGGSGGS

SEQ ID NO: 203 - skipped

SEQ ID NO: 204 - protein, 40 aa, synthetic construct
  REGION 1..40: note = Description of Artificial Sequence: Synthetic polypeptide
  REGION 1..40: note = MISC_FEATURE - This sequence may encompass 1-20 "Gly Ser" repeating units
GSGSGSGSGSGSGSGSGSGSGSGSGSGSGSGSGSGSGSGS

SEQ ID NO: 205 - protein, 60 aa, synthetic construct
  REGION 1..60: note = Description of Artificial Sequence: Synthetic polypeptide
  REGION 1..60: note = MISC_FEATURE - This sequence may encompass 1-20 "Gly Gly Ser" repeating units
GGSGGSGGSGGSGGSGGSGGSGGSGGSGGSGGSGGSGGSGGSGGSGGSGGSGGSGGSGGS

SEQ ID NO: 206 - protein, 80 aa, synthetic construct
  REGION 1..80: note = Description of Artificial Sequence: Synthetic polypeptide
  REGION 1..80: note = MISC_FEATURE - This sequence may encompass 1-20 "Gly Gly Gly Ser" repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 207 - protein, 80 aa, synthetic construct
  REGION 1..80: note = Description of Artificial Sequence: Synthetic polypeptide
  REGION 1..80: note = MISC_FEATURE - This sequence may encompass 1-20 "Gly Gly Ser Gly" repeating units
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 208 - protein, 100 aa, synthetic construct; identical to SEQ ID NO: 154
  REGION 1..100: note = Description of Artificial Sequence: Synthetic polypeptide
  REGION 1..100: note = MISC_FEATURE - This sequence may encompass 1-20 "Gly Gly Ser Gly Gly" repeating units

SEQ ID NO: 209 - protein, 100 aa, synthetic construct; identical to SEQ ID NO: 152
  REGION 1..100: note = Description of Artificial Sequence: Synthetic polypeptide
  REGION 1..100: note = MISC_FEATURE - This sequence may encompass 1-20 "Gly Gly Gly Gly Ser" repeating units

(19) United States
(12) Patent Application Publication
(10) Pub. No.: US 2025/0206827 A1
(43) Pub. Date: Jun. 26, 2025

(54) MULTI-SPECIFIC BINDING PROTEINS THAT BIND HER2, NKG2D, AND CD16, AND METHODS OF USE

(71) Applicant: Dragonfly Therapeutics, Inc., Waltham, MA (US)

(72) Inventors: Gregory P. Chang, Medford, MA (US); Ann F. Cheung, Lincoln, MA (US); Daniel Fallon, Winchester, MA (US); Asya Grinberg, Lexington, MA (US); William Haney, Wayland, MA (US); Steven O'Neil, Wayland, MA (US); Nicolai Wagtmann, Concord, MA (US); Ronnie Wei, Weston, MA (US); Bradley M. Lunde, Lebanon, NH (US); Bianka Prinz, Lebanon, NH (US)

(21) Appl. No.: 19/076,034

(22) Filed: Mar. 11, 2025

Related U.S. Application Data
(63) Continuation of application No. 17/265,876, filed on Feb. 4, 2021, now Pat. No. 12,275,791.
(60) Provisional application No. 62/716,259, filed on Aug. 8, 2018.

(51) Int. Cl. C07K 16/28; C07K 16/32

(52) U.S. Cl. CPC C07K 16/2851; C07K 16/283; C07K 16/32; C07K 2317/21; C07K 2317/31; C07K 2317/55; C07K 2317/622; C07K 2317/73; C07K 2317/92

(57) ABSTRACT

The present disclosure provides multi-specific binding proteins that bind to and kill human cancer cells expressing epidermal growth factor receptor 2 (HER2 or ErbB2), but does not kill non-cancerous healthy human cells expressing HER2 are described, as well as pharmaceutical compositions and therapeutic methods useful for the treatment of HER2 expressing cancer.

### CROSS-REFERENCE TO RELATED APPLICATIONS

This application is a continuation of U.S. patent application Ser. No. 17/265,876, filed on Feb. 4, 2021, which is a U.S. national stage application, filed under 35 U.S.C. § 371, of International Application No. PCT/US2019/045561, filed on Aug. 7, 2019, which claims the benefit of and priority to U.S. Provisional Patent Application No. 62/716,259, filed on Aug. 8, 2018, the disclosure of each of which is hereby incorporated by reference in its entirety for all purposes.

### SEQUENCE LISTING

The instant application contains a Sequence Listing which has been submitted electronically in XML format and is hereby incorporated by reference in its entirety. Said XML copy, created on Mar. 4, 2025, is named DFY-057USC1_ST26.xml and is 288,382 bytes in size.

### FIELD OF THE INVENTION

The invention relates to multi-specific binding proteins that bind to NKG2D, CD16, and epidermal growth factor receptor 2 (HER2 or ErbB2). These multi-specific binding proteins are useful for killing human cancer cells expressing Her2 without significant cytotoxicity to non-cancerous healthy human cells expressing HER2.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, e.g., WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-γ and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals.

HER2 (ErbB2) is a transmembrane glycoprotein, which belongs to the epidermal growth factor receptor family. It is a receptor tyrosine kinase and regulates cell survival, proliferation, and growth. HER2 plays an important role in human malignancies. The ERBB2 gene is amplified or overexpressed in approximately 30% of human breast cancers. Patients with HER2-overexpressing breast cancer have substantially lower overall survival rates and shorter disease-free intervals than patients whose cancer does not overexpress HER2. Moreover, overexpression of HER2 leads to increased breast cancer metastasis. Over-expression of HER2 is also known to occur in many other cancer types, including breast, ovarian, esophageal, bladder and gastric cancer, salivary duct carcinoma, adenocarcinoma of the lung and aggressive forms of uterine cancer, such as uterine serous endometrial carcinoma.

### SUMMARY

The invention relates to multi-specific binding proteins that bind to and kill human cancer cells expressing epidermal growth factor receptor 2 (HER2 or ErbB2). The invention provides multi-specific binding proteins that bind to HER2 on a cancer cell and to the NKG2D receptor and CD16 receptor on natural killer cells. Such proteins can engage more than one kind of NK activating receptor, and may block the binding of natural ligands to NKG2D. In certain embodiments, the proteins can agonize NK cells in humans, and in other species such as rodents and cynomolgus monkeys. In certain embodiments, the proteins can stimulate T cells in humans, and in other species such as human, rodents and cynomolgus monkeys. Various aspects and embodiments of the invention are described in further detail below.

In one aspect, the present invention provides a protein (e.g., a multi-specific binding protein) comprising: (a) a first antigen-binding site comprising an Fab fragment that binds NKG2D; (b) a second antigen-binding site comprising a single-chain variable fragment (scFv) that binds HER2; and (c) an antibody Fc domain or a portion thereof sufficient to bind CD16, or a third antigen-binding site that binds CD16.

In certain embodiments, the scFv is linked to the antibody Fc domain or a portion thereof sufficient to bind CD16, or the third antigen-binding site that binds CD16, via a hinge comprising Ala-Ser. In certain embodiments, the scFv is linked to the antibody Fc domain.

In certain embodiments, the scFv comprises a heavy chain variable domain and a light chain variable domain. In certain embodiments, the heavy chain variable domain of the scFv forms a disulfide bridge with the light chain variable domain of the scFv. In certain embodiments, the disulfide bridge is formed between C44 of the heavy chain variable domain and C100 of the light chain variable domain.

In certain embodiments, the light chain variable domain of the scFv is linked to the heavy chain variable domain of the scFv via a flexible linker. In certain embodiments, the flexible linker comprises the amino acid sequence of SEQ ID NO:143. In certain embodiments, the flexible linker consists of the amino acid sequence of SEQ ID NO:143. In certain embodiments, the light chain variable domain is positioned to the N-terminus or C-terminus to the heavy chain variable domain. In certain embodiments, the light chain variable domain is positioned to the N-terminus of the heavy chain variable domain.

In certain embodiments, the Fab fragment is linked to the antibody Fc domain or a portion thereof sufficient to bind CD16, or the third antigen-binding site that binds CD16.

In certain embodiments, the first antigen-binding site that binds NKG2D comprises a heavy chain variable domain comprising complementarity-determining region 1 (CDR1), complementarity-determining region 2 (CDR2), and complementarity-determining region 3 (CDR3) sequences represented by the amino acid sequences of SEQ ID NOs: 168, 96, and 169, respectively; and a light chain variable domain comprising CDR1, CDR2, and CDR3 sequences represented by the amino acid sequences of SEQ ID NOs: 99, 100, and 101, respectively. In certain embodiments, the first antigen-binding site that binds NKG2D comprises a heavy chain variable domain comprising CDR1, CDR2, and CDR3 sequences represented by the amino acid sequences of SEQ ID NOs: 95, 96, and 97, respectively; and a light chain variable domain comprising CDR1, CDR2, and CDR3 sequences represented by the amino acid sequences of SEQ ID NOs: 99, 100, and 101, respectively.

In certain embodiments, a protein of the present invention comprising a first antigen-binding that binds NKG2D, comprises:
(a) a heavy chain variable domain comprising complementarity-determining region 1 (CDR1), complementarity-determining region 2 (CDR2), and complementarity-determining region 3 (CDR3) sequences represented by the amino acid sequences of SEQ ID NOs: 168, 96, and 169, respectively; and a light chain variable domain comprising CDR1, CDR2, and CDR3 sequences represented by the amino acid sequences of SEQ ID NOs: 99, 100, and 101, respectively;
(b) a heavy chain variable domain comprising CDR1, CDR2, and CDR3 sequences represented by the amino acid sequences of SEQ ID NOs: 168, 96, and 173, respectively; and a light chain variable domain comprising CDR1, CDR2, and CDR3 sequences represented by the amino acid sequences of SEQ ID NOs: 99, 100, and 101, respectively;
(c) a heavy chain variable domain comprising CDR1, CDR2, and CDR3 sequences represented by the amino acid sequences of SEQ ID NOs: 95, 96, and 97, respectively; and a light chain variable domain comprising CDR1, CDR2, and CDR3 sequences represented by the amino acid sequences of SEQ ID NOs: 99, 100, and 101, respectively;
(d) a heavy chain variable domain comprising CDR1, CDR2, and CDR3 sequences represented by the amino acid sequences of SEQ ID NOs: 166, 88, and 167, respectively; and a light chain variable domain comprising CDR1, CDR2, and CDR3 sequences represented by the amino acid sequences of SEQ ID NOs: 91, 92, and 93, respectively;
(e) a heavy chain variable domain comprising CDR1, CDR2, and CDR3 sequences represented by the amino acid sequences of SEQ ID NOs: 162, 72, and 170, respectively; and a light chain variable domain comprising CDR1, CDR2, and CDR3 sequences represented by the amino acid sequences of SEQ ID NOs: 107, 108, and 109, respectively;
(f) a heavy chain variable domain comprising CDR1, CDR2, and CDR3 sequences represented by the amino acid sequences of SEQ ID NOs: 162, 72, and 163, respectively; and a light chain variable domain comprising CDR1, CDR2, and CDR3 sequences represented by the amino acid sequences of SEQ ID NOs: 75, 76, and 77, respectively;
(g) a heavy chain variable domain comprising CDR1, CDR2, and CDR3 sequences represented by the amino acid sequences of SEQ ID NOs: 164, 80, and 165, respectively; and a light chain variable domain comprising CDR1, CDR2, and CDR3 sequences represented by the amino acid sequences of SEQ ID NOs: 75, 76, and 85, respectively;
(h) a heavy chain variable domain comprising CDR1, CDR2, and CDR3 sequences represented by the amino acid sequences of SEQ ID NOs: 168, 96, and 176, respectively; and a light chain variable domain comprising CDR1, CDR2, and CDR3 sequences represented by the amino acid sequences of SEQ ID NOs: 99, 100, and 101, respectively;
(i) a heavy chain variable domain comprising CDR1, CDR2, and CDR3 sequences represented by the amino acid sequences of SEQ ID NOs: 168, 96, and 179, respectively; and a light chain variable domain comprising CDR1, CDR2, and CDR3 sequences represented by the amino acid sequences of SEQ ID NOs: 99, 100, and 101, respectively;
(j) a heavy chain variable domain comprising CDR1, CDR2, and CDR3 sequences represented by the amino acid sequences of SEQ ID NOs: 168, 96, and 182, respectively; and a light chain variable domain comprising CDR1, CDR2, and CDR3 sequences represented by the amino acid sequences of SEQ ID NOs: 99, 100, and 101, respectively;
(k) a heavy chain variable domain comprising CDR1, CDR2, and CDR3 sequences represented by the amino acid sequences of SEQ ID NOs: 168, 96, and 185, respectively; and a light chain variable domain comprising CDR1, CDR2, and CDR3 sequences represented by the amino acid sequences of SEQ ID NOs: 99, 100, and 101, respectively; or
(l) a heavy chain variable domain comprising CDR1, CDR2, and CDR3 sequences represented by the amino acid sequences of SEQ ID NOs: 168, 96, and 188, respectively; and a light chain variable domain comprising CDR1, CDR2, and CDR3 sequences represented by the amino acid sequences of SEQ ID NOs: 99, 100, and 101, respectively; and a second antigen-binding site comprising an scFv that binds HER2, comprises:
(a) a heavy chain variable domain comprising CDR1, CDR2, and CDR3 sequences represented by the amino acid sequences of SEQ ID NOs: 115, 116, and 117, respectively, and a light chain variable domain comprising CDR1, CDR2, and CDR3 sequences represented by the amino acid sequences of SEQ ID NOs: 119, 120, and 121, respectively;
(b) a heavy chain variable domain comprising CDR1, CDR2, and CDR3 sequences represented by the amino acid sequences of SEQ ID NOs: 123, 124, and 125, respectively, and a light chain variable domain comprising CDR1, CDR2, and CDR3 sequences represented by the amino acid sequences of SEQ ID NOs: 127, 128, and 129, respectively; or
(c) a heavy chain variable domain comprising CDR1, CDR2, and CDR3 sequences represented by the amino acid sequences of SEQ ID NOs: 131, 132, and 133, respectively, and a light chain variable domain comprising CDR1, CDR2, and CDR3 sequences represented by the amino acid sequences of SEQ ID NOs: 135, 136, and 137, respectively.

In some embodiments, the first antigen-binding site comprises a heavy chain variable domain related to SEQ ID NO:94 and a light chain variable domain related to SEQ ID NO: 98. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:94, and/or incorporate amino acid sequences identical to the CDR1 (SEQ ID NO:95 or 168), CDR2 (SEQ ID NO:96), and CDR3 (SEQ ID NO:97 or 169) sequences of SEQ ID NO:94. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO: 101) sequences of SEQ ID NO:98.

In some embodiments, the first antigen-binding site comprises a heavy chain variable domain related to SEQ ID NO:144 and a light chain variable domain related to SEQ ID NO: 98. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:144, and/or incorporate amino acid sequences identical to the CDR1 (SEQ ID NO:95 or 168), CDR2 (SEQ ID NO:96), and CDR3 (SEQ ID NO:172 or 173) sequences of SEQ ID NO:144. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO: 101) sequences of SEQ ID NO:98.

In some embodiments, the first antigen-binding site comprises a heavy chain variable domain related to SEQ ID NO:174 and a light chain variable domain related to SEQ ID NO: 98. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:174, and/or incorporate amino acid sequences identical to the CDR1 (SEQ ID NO:95 or 168), CDR2 (SEQ ID NO:96), and CDR3 (SEQ ID NO:175 or 176) sequences of SEQ ID NO:174. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO: 101) sequences of SEQ ID NO:98.

In some embodiments, the first antigen-binding site comprises a heavy chain variable domain related to SEQ ID NO:177 and a light chain variable domain related to SEQ ID NO: 98. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:177, and/or incorporate amino acid sequences identical to the CDR1 (SEQ ID NO:95 or 168), CDR2 (SEQ ID NO:96), and CDR3 (SEQ ID NO:178 or 179) sequences of SEQ ID NO:177. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO: 101) sequences of SEQ ID NO:98.

In some embodiments, the first antigen-binding site comprises a heavy chain variable domain related to SEQ ID NO:180 and a light chain variable domain related to SEQ ID NO: 98. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:180, and/or incorporate amino acid sequences identical to the CDR1 (SEQ ID NO:95 or 168), CDR2 (SEQ ID NO:96), and CDR3 (SEQ ID NO:181 or 182) sequences of SEQ ID NO:180. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO: 101) sequences of SEQ ID NO:98.

In some embodiments, the first antigen-binding site comprises a heavy chain variable domain related to SEQ ID NO:183 and a light chain variable domain related to SEQ ID NO: 98. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:183, and/or incorporate amino acid sequences identical to the CDR1 (SEQ ID NO:95 or 168), CDR2 (SEQ ID NO:96), and CDR3 (SEQ ID NO:184 or 185) sequences of SEQ ID NO:183. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO: 101) sequences of SEQ ID NO:98.

In some embodiments, the first antigen-binding site comprises a heavy chain variable domain related to SEQ ID NO:186 and a light chain variable domain related to SEQ ID NO: 98. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:186, and/or incorporate amino acid sequences identical to the CDR1 (SEQ ID NO:95 or 168), CDR2 (SEQ ID NO:96), and CDR3 (SEQ ID NO:187 or 188) sequences of SEQ ID NO:186. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO: 101) sequences of SEQ ID NO:98.

In some embodiments, the first antigen-binding site comprises a heavy chain variable domain related to SEQ ID NO:86 and a light chain variable domain related to SEQ ID NO: 90. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:87 or 166), CDR2 (SEQ ID NO:88), and CDR3 (SEQ ID NO:89 or 167) sequences of SEQ ID NO:86. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:90, and/or incorporate amino acid sequences identical to the CDR1 (SEQ ID NO:91), CDR2 (SEQ ID NO:92), and CDR3 (SEQ ID NO: 93) sequences of SEQ ID NO:90.

In some embodiments, the first antigen-binding site comprises a heavy chain variable domain related to SEQ ID NO:102 and a light chain variable domain related to SEQ ID NO: 106. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:102, and/or incorporate amino acid sequences identical to the CDR1 (SEQ ID NO:71 or 162), CDR2 (SEQ ID NO:72), and CDR3 (SEQ ID NO:105 or 170) sequences of SEQ ID NO:102. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:106, and/or incorporate amino acid sequences identical to the CDR1 (SEQ ID NO:107), CDR2 (SEQ ID NO:108), and CDR3 (SEQ ID NO: 109) sequences of SEQ ID NO:106.

In some embodiments, the first antigen-binding site comprises a heavy chain variable domain related to SEQ ID NO:70 and a light chain variable domain related to SEQ ID NO: 74. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:71 or 162), CDR2 (SEQ ID NO:72), and CDR3 (SEQ ID NO:73 or 163) sequences of SEQ ID NO:70. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:74, and/or incorporate amino acid sequences identical to the CDR1 (SEQ ID NO:75), CDR2 (SEQ ID NO:76), and CDR3 (SEQ ID NO: 77) sequences of SEQ ID NO:74.

In some embodiments, the first antigen-binding site comprises a heavy chain variable domain related to SEQ ID NO:70 and a light chain variable domain related to SEQ ID NO: 74. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:71 or 162), CDR2 (SEQ ID NO:72), and CDR3 (SEQ ID NO:73 or 163) sequences of SEQ ID NO:70. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:74, and/or incorporate amino acid sequences identical to the CDR1 (SEQ ID NO:75), CDR2 (SEQ ID NO:76), and CDR3 (SEQ ID NO: 77) sequences of SEQ ID NO:74.

In some embodiments, the first antigen-binding site comprises a heavy chain variable domain related to SEQ ID NO:78 and a light chain variable domain related to SEQ ID NO: 82. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:78, and/or incorporate amino acid sequences identical to the CDR1 (SEQ ID NO:79 or 164), CDR2 (SEQ ID NO:80), and CDR3 (SEQ ID NO:81 or 165) sequences of SEQ ID NO:78. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:82, and/or incorporate amino acid sequences identical to the CDR1 (SEQ ID NO:75), CDR2 (SEQ ID NO:76), and CDR3 (SEQ ID NO: 77) sequences of SEQ ID NO:82.

In certain embodiments, the first antigen-binding site binds to NKG2D with a K_{D} of 2 nM to 120 nM, as measured by surface plasmon resonance. In certain embodiments, the protein binds to NKG2D with a K_{D} of 2 nM to 120 nM, as measured by surface plasmon resonance.

In some embodiments, the second antigen-binding site binding to HER2 comprises a heavy chain variable domain related to SEQ ID NO:195 and a light chain variable domain related to SEQ ID NO:196. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:195, and/or incorporate amino acid sequences identical to the CDR1 (SEQ ID NO:115), CDR2 (SEQ ID NO:116), and CDR3 (SEQ ID NO:117) sequences of SEQ ID NO:195. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:196, and/or incorporate amino acid sequences identical to the CDR1 (SEQ ID NO:119), CDR2 (SEQ ID NO:120), and CDR3 (SEQ ID NO:121) sequences of SEQ ID NO:196. In some embodiments, the second antigen-binding site binding to HER2 comprises an scFv comprising the amino acid sequence of SEQ ID NO:139.

Alternatively, the second antigen-binding site binding to HER2 comprises a heavy chain variable domain related to SEQ ID NO:197 and a light chain variable domain related to SEQ ID NO:198. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:197, and/or incorporate amino acid sequences identical to the CDR1 (SEQ ID NO:123), CDR2 (SEQ ID NO:124), and CDR3 (SEQ ID NO: 125) sequences of SEQ ID NO:197. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:198, and/or incorporate amino acid sequences identical to the CDR1 (SEQ ID NO:127), CDR2 (SEQ ID NO:128), and CDR3 (SEQ ID NO:129) sequences of SEQ ID NO:198. In some embodiments, the second antigen-binding site binding to HER2 comprises an scFv comprising the amino acid sequence of SEQ ID NO: 189.

Alternatively, the second antigen-binding site binding to HER2 comprises a heavy chain variable domain related to SEQ ID NO:199 and a light chain variable domain related to SEQ ID NO:200. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:199, and/or incorporate amino acid sequences identical to the CDR1 (SEQ ID NO:131), CDR2 (SEQ ID NO:132), and CDR3 (SEQ ID NO: 133) sequences of SEQ ID NO:199. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:200, and/or incorporate amino acid sequences identical to the CDR1 (SEQ ID NO:135), CDR2 (SEQ ID NO:136), and CDR3 (SEQ ID NO:137) sequences of SEQ ID NO:200. In some embodiments, the second antigen-binding site binding to HER2 comprises an scFv comprising the amino acid sequence of SEQ ID NO: 171.

In certain embodiments, the antibody Fc domain comprises hinge and CH2 domains of a human IgG1 antibody. In certain embodiments, the antibody Fc domain comprises an amino acid sequence at least 90% identical to amino acids 234-332 of a human IgG1 antibody.

In certain embodiments, the antibody Fc domain comprises an Fc domain comprising an amino acid sequence at least 90% identical to the Fc domain of human IgG1 and differs at one or more positions selected from the group consisting of Q347, Y349, T350, L351, S354, E356, E357, K360, Q362, S364, T366, L368, K370, N390, K392, T394, D399, S400, D401, F405, Y407, K409, T411, K439.

In certain embodiments, the antibody Fc domain comprises an Fc domain of a human IgG1 comprising Q347R, D399V, and F405T substitutions, e.g., in an Fc domain linked to an scFv. In certain embodiments, the antibody Fc domain comprises an Fc domain of a human IgG1 comprising K360E and K409W substitutions, e.g., in an Fc domain linked to an Fab fragment.

In certain embodiments, the antibody Fc domain comprises an Fc domain of a human IgG1 comprising a T366W substitution, e.g., in an Fc domain linked to an Fab fragment. In certain embodiments, the antibody Fc domain comprises an Fc domain of a human IgG1 comprising T366S, L368A, and Y407V substitutions, e.g., in an Fc domain linked to an scFv.

In certain embodiments, the protein comprises a sequence of SEQ ID NO:141, SEQ ID NO:145, SEQ ID NO:147, SEQ ID NO:194, SEQ ID NO: 155, or SEQ ID NO:148. In certain embodiments, this sequence represents the heavy chain portion of the Fab fragment linked to the antibody Fc domain.

In certain embodiments, the protein comprises a sequence of SEQ ID NO:140 or SEQ ID NO:146. In certain embodiments, this sequence represents the scFv linked to the antibody Fc domain.

In another aspect, the instant disclosure provides a protein (e.g., a multi-specific binding protein) comprising: (a) a first polypeptide comprising an amino acid sequence at least 90% (e.g., 90%, 91%, 92%, 93%, 94%, 95%, 96%, 97%, 98%, 99%, or 100%) identical to SEQ ID NO:141; (b) a second polypeptide comprising an amino acid sequence at least 90% (e.g., 90%, 91%, 92%, 93%, 94%, 95%, 96%, 97%, 98%, 99%, or 100%) identical to SEQ ID NO:140, SEQ ID NO:190, or SEQ ID NO: 192; and (c) a third polypeptide comprising an amino acid sequence at least 90% (e.g., 90%, 91%, 92%, 93%, 94%, 95%, 96%, 97%, 98%, 99%, or 100%) identical to SEQ ID NO:142. In certain embodiments, the second polypeptide comprises an amino acid sequence at least 90% (e.g., 90%, 91%, 92%, 93%, 94%, 95%, 96%, 97%, 98%, 99%, or 100%) identical to SEQ ID NO:140.

In another aspect, the instant disclosure provides a protein (e.g., a multi-specific binding protein) comprising: (a) a first polypeptide comprising an amino acid sequence at least 95% (e.g., 95%, 96%, 97%, 98%, 99%, or 100%) identical to SEQ ID NO:141; (b) a second polypeptide comprising an amino acid sequence at least 95% (e.g., 95%, 96%, 97%, 98%, 99%, or 100%) identical to SEQ ID NO:140, SEQ ID NO:190, or SEQ ID NO: 192; and (c) a third polypeptide comprising an amino acid sequence at least 95% (e.g., 95%, 96%, 97%, 98%, 99%, or 100%) identical to SEQ ID NO:142. In certain embodiments, the second polypeptide comprises an amino acid sequence at least 95% (e.g., 95%, 96%, 97%, 98%, 99%, or 100%) identical to SEQ ID NO:140.

In another aspect, the instant disclosure provides a protein (e.g., a multi-specific binding protein) comprising: (a) a first polypeptide comprising the amino acid sequence of SEQ ID NO:141; (b) a second polypeptide comprising the amino acid sequence of SEQ ID NO: 140, SEQ ID NO:190, or SEQ ID NO:192; and (c) a third polypeptide comprising the amino acid sequence of SEQ ID NO:142. In certain embodiments, the protein comprises (a) a first polypeptide comprising the amino acid sequence of SEQ ID NO:141; (b) a second polypeptide comprising the amino acid sequence of SEQ ID NO:140; and (c) a third polypeptide comprising the amino acid sequence of SEQ ID NO:142.

In another aspect, the instant disclosure provides a formulation comprising a protein disclosed herein and a pharmaceutically acceptable carrier.

In another aspect, the instant disclosure provides a cell comprising one or more nucleic acids expressing a protein disclosed herein.

In another aspect, the instant disclosure provides a method of directly and/or indirectly enhancing tumor cell death, the method comprising exposing a tumor and natural killer cells to a protein disclosed herein.

In another aspect, the instant disclosure provides a method of treating cancer, wherein the method comprises administering a protein or a formulation disclosed herein to a patient in need thereof. In certain embodiments, the cancer is selected from the group consisting of breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, lung squamous, mesothelioma, liver cancer, mesothelioma, sarcoma, and gall bladder cancer.

### BRIEF DESCRIPTION OF THE DRAWINGS

FIG. 1 illustrates a trispecific antibody (TriNKET) that contains an HER2-binding scFv, a NKG2D-targeting Fab, and a heterodimerized antibody constant region/domain (“CD domain”) that binds CD16 (scFv-Fab format). In an exemplary embodiment, the Fc domain linked to the Fab fragment comprises mutations of K360E and K409W and the Fc domain linked to the scFv comprises matching mutations Q347R, D399V, and F405T for forming an Fc heterodimer (shown as a triangular lock-and-key format in the Fc domains in FIG. 1). The antibody format is referred herein as F3′-TriNKET. In another exemplary embodiment, the Fc domain linked to the Fab fragment comprises the mutations of Q347R, D399V, and F405T, and the Fc domain linked to the scFv comprises matching mutations K360E and K409W for forming a heterodimer.

FIG. 2 is a representation of a “knob-in-hole” (KiH) TriNKET, which includes an scFv that binds HER2 linked to an Fc domain via a hinge comprising Ala-Ser, and an NKG2D-binding Fab fragment including (a) a heavy chain portion comprising a heavy chain variable domain and a CH1 domain connected to an Fc domain, and (b) a light chain portion comprising a light chain variable domain and a CL domain. In an exemplary embodiment, the Fc domain linked to the Fab fragment comprises a knob mutation T366W, and the Fc domain linked to the scFv comprises matching “hole” mutations T366S, L368A, Y407V (shown as a triangular lock-and-key format in the Fc domains in FIG. 2). In an exemplary embodiment, the Fc domain linked to the Fab fragment comprises knob mutations T366S, L368A, Y407V, and the Fc domain linked to the scFv comprises a “hole” mutation T366W.

FIG. 3 are line graphs demonstrating HER2-targeted TriNKETs are more potent than trastuzumab on a HER2+ (low) cell line.

FIG. 4 are line graphs demonstrating HER2-targeted TriNKETs are more potent than trastuzumab on a HER2++ cell line.

FIG. 5 are line graphs demonstrating HER2-targeted TriNKETs are more potent than trastuzumab on a HER2+++ cell line.

FIG. 6 shows HER2-targeted TriNKETs outperform trastuzumab in a long-term killing assay.

FIG. 7 shows HER2-targeted TriNKETs outperform trastuzumab in a long-term killing assay.

FIGS. 8A to 8F are FACS showing that HER2-Targeted TriNKETs show minimal binding to immune cells in human blood. FIG. 8A shows that HER2-Targeted TriNKETs show minimal binding to NK cells in human blood; FIG. 8B shows that HER2-Targeted TriNKETs show minimal binding to CD8+ T cells; FIG. 8C shows that HER2-Targeted TriNKETs show minimal binding to CD4⁺ T cells; FIG. 8D shows that HER2-Targeted TriNKETs show minimal binding to B cells; FIG. 8E shows that HER2-Targeted TriNKETs show minimal binding to monocytes; and FIG. 8F shows that HER2-Targeted TriNKETs show minimal binding to granulocytes (Dash-dotted line-secondary control; dashed line-Trastuzumab; Solid line-HER2-F3′-TriNKET-A49).

FIG. 9 are line graphs demonstrating binding of A49-F3′-TriNKET-Trastuzumab to human cardiomyocytes, SKBR3, H661 and 786-O cancer cells.

FIG. 10A shows that A49-F3′-TriNKET-Trastuzumab-mediated human PBMC killing of SKBR3 cancer after 3 days in co-culture at PBMC to target cell ratio (E:T) of 1:1.

FIG. 10B shows that A49-F3′-TriNKET-Trastuzumab does not kill non-malignant healthy cardiomyocytes even after 3 days in co-culture at PBMC to target cell ratio (E:T) of 1:1.

FIG. 11A shows A49-F3′-TriNKET-Trastuzumab-mediated human PBMC killing of SKBR3 cancer cells after 3 days in co-culture at PBMC to target cell ratio (E:T) of 20:1.

FIG. 11B shows that A49-F3′-TriNKET-Trastuzumab does not kill non-malignant healthy cardiomyocytes even after 3 days in co-culture at PBMC to target cell ratio (E:T) of 20:1.

FIGS. 12A to 12B show that CD8+ T cells generated with conA stimulation and cultured with IL-15 were of high purity (99% of CD3+CD8+ cells) (FIG. 12A), and all expressed NKG2D (FIG. 12B), but not CD16 (FIG. 12C).

FIGS. 13A to 13B are graphs showing cytotoxic activity of CD8+ T cells in the presence of A49-F3′-TriNKET-Trastuzumab, after culturing with IL-15. FIG. 13A shows enhanced killing of SkBr-3 tumor cells in short-term co-cultures by A49-F3′-TriNKET-Trastuzumab. HER2-targeting A49-F3′-TriNKET-Trastuzumab triggered dose-dependent lysis of SkBr-3 target cells by IL-15 stimulated CD8+ T cell. FIG. 13B shows that 67 nM of HER2-targeting TriNKET triggered lysis of SkBr-3 target cells by IL-2 stimulated CD8+ T cell. Dotted line indicates the effect with only CD8+ T cells co-cultured with SkBr-3 tumor cells (untreated).

FIG. 14A shows percent growth levels of SkBr-3 cells cultured alone, co-cultured with CD8+ T cells, co-cultured with CD8+ T cells and A49-F3′-TriNKET-Trastuzumab, each in the presence of anti-CD3.

FIG. 14B shows percent growth level of SkBr-3 cells cultured alone, co-cultured with CD8+ T cells, co-cultured with CD8+ T cells and A49-F3′-TriNKET-Trastuzumab, each in the absence of anti-CD3.

FIGS. 15A to 15B show TriNKET binding to SkBr-3 cell line that has a high level of HER2 expression, as measured by flow cytometry of SkBr-3 cells incubated with a series of concentrations of TriNKET or trastuzumab and a secondary antibody conjugated with a fluorophore. FIG. 15A shows the level of binding as percentage values of median fluorescence intensity (MFI) relative to the maximum MFI observed with the cells incubated with 670 nM of TriNKET. FIG. 15B shows the level of binding as fold over background (FOB) values of MFI relative to the background MFI observed with the cells incubated with the secondary antibody only.

FIGS. 16A to 16B show TriNKET binding to NCI-H661 cell line that has a moderate level of HER2 expression, as measured by flow cytometry of NCI-H661 cells incubated with a series of concentrations of TriNKET or trastuzumab and a secondary antibody conjugated with a fluorophore. FIG. 16A shows the level of binding as percentage values of median fluorescence intensity (MFI) relative to the maximum MFI observed with the cells incubated with 670 nM of TriNKET. FIG. 16B shows the level of binding as fold over background (FOB) values of MFI relative to the background MFI observed with the cells incubated with the secondary antibody only.

FIGS. 17A to 17B show TriNKET binding to 786-O cell line that has a low level of HER2 expression, as measured by flow cytometry of 786-O cells incubated with a series of concentrations of TriNKET or trastuzumab and a secondary antibody conjugated with a fluorophore. FIG. 16A shows the level of binding as percentage values of median fluorescence intensity (MFI) relative to the maximum MFI observed with the cells incubated with 670 nM of TriNKET. FIG. 16B shows the level of binding as fold over background (FOB) values of MFI relative to the background MFI observed with the cells incubated with the secondary antibody only.

FIGS. 18A to 18B show HER2-targeted TriNKETs binding to hNKG2D-expressing EL4 cells, as measured by flow cytometry of EL4 cells incubated with a series of concentrations of TriNKETs or trastuzumab and a secondary antibody conjugated with a fluorophore. The levels of binding are shown as fold over background (FOB) values of MFI relative to the background MFI observed with the cells incubated with the secondary antibody only.

FIG. 19 shows TriNKETs are more potent and effective in mediating NK-cell killing of 786-O target cells than the combination of Fc-silent TriNKET and trastuzumab.

FIG. 20 shows TriNKETs are more potent and effective in mediating NK-cell killing of H661 target cells than the combination of Fc-silent TriNKET and trastuzumab.

FIG. 21 is a line graph showing the potency of TriNKET A and TriNKET A*(in which M102 of A49-F3′-TriNKET-Trastuzumab is substituted with I) in mediating cytotoxicity of NK cells against SKBR-3 target cells.

### DETAILED DESCRIPTION

The invention provides multi-specific binding proteins that bind HER2 on a cancer cell and the NKG2D receptor and CD16 receptor on natural killer cells, pharmaceutical compositions comprising such multi-specific binding proteins, and therapeutic methods using such multi-specific proteins and pharmaceutical compositions, including for the treatment of cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering.

The term “tumor-associated antigen” as used herein means any antigen including but not limited to a protein, glycoprotein, ganglioside, carbohydrate, or lipid that is associated with cancer. Such an antigen can be expressed on malignant cells or in the tumor microenvironment, such as on tumor-associated blood vessels, extracellular matrix, mesenchymal stroma, or immune infiltrates.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. PROTEINS

The invention provides multi-specific binding proteins that bind HER2 on a cancer cell and the NKG2D receptor and CD16 receptor on natural killer cells to activate the natural killer cell. The multi-specific binding proteins are useful in the pharmaceutical compositions and therapeutic methods described herein. Binding of the multi-specific binding protein to the NKG2D receptor and CD16 receptor on natural killer cell enhances the activity of the natural killer cell toward destruction of a cancer cell. Binding of the multi-specific binding protein to HER2 on a cancer cell brings the cancer cell into proximity with the natural killer cell, which facilitates direct and indirect destruction of the cancer cell by the natural killer cell. Further description of exemplary multi-specific binding proteins is provided below.

The first component of the multi-specific binding proteins binds to NKG2D receptor-expressing cells, which can include but are not limited to NK cells, NKT cells, γδ T cells and CD8+αβ T cells. Upon NKG2D binding, the multi-specific binding proteins may block natural ligands, such as ULBP6 and MICA, from binding to NKG2D and activating NK cells.

The second component of the multi-specific binding proteins binds to HER2-expressing cells, which can include but are limited to breast, ovarian, esophageal, bladder and gastric cancer, salivary duct carcinoma, adenocarcinoma of the lung and aggressive forms of uterine cancer, such as uterine serous endometrial carcinoma.

The third component for the multi-specific binding proteins binds to cells expressing CD16, an Fc receptor on the surface of leukocytes including natural killer cells, macrophages, neutrophils, eosinophils, mast cells, and follicular dendritic cells.

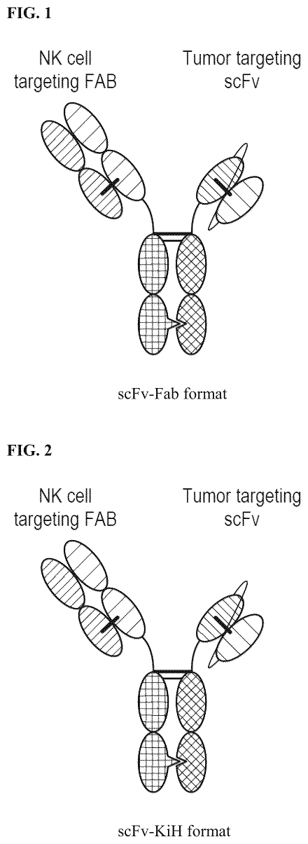

The multi-specific binding proteins described herein can take various formats. For example, one format involves a heterodimeric, multi-specific antibody including a first immunoglobulin heavy chain, a second immunoglobulin heavy chain and an immunoglobulin light chain (FIG. 1). The first immunoglobulin heavy chain includes a first Fc (hinge-CH2-CH3) domain fused via either a linker or an antibody hinge to an Fab fragment composed of a heavy chain portion comprising a heavy chain variable domain and a heavy chain CH1 domain, and a light chain portion comprising a light chain variable domain and a light chain constant domain (CL), wherein the heavy chain and light chain portions of the Fab fragment pair and bind NKG2D. The second immunoglobulin heavy chain includes a second Fc (hinge-CH2-CH3) domain fused via either a linker or an antibody hinge to a single-chain variable fragment (scFv) composed of a heavy chain variable domain and light chain variable domain which pair and bind the HER2 antigen.

In some embodiments, the single-chain variable fragment (scFv) described above is linked to the antibody constant domain via a hinge sequence. In some embodiments, the hinge comprises amino acids Ala-Ser. In some other embodiments, the hinge comprises amino acids Ala-Ser and Thr-Lys-Gly. The hinge sequence can provide flexibility of binding to the target antigen, and balance between flexibility and optimal geometry.

In some embodiments, the single-chain variable fragment (scFv) described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. In some embodiments, the heavy chain variable domain is linked to the light chain variable domain via a flexible linker. Any suitable linker can be used, for example, the (G4S)₄ linker (SEQ ID NO: 143). In some embodiments of the scFv, the heavy chain variable domain is positioned at the N-terminus of the light chain variable domain. In some embodiments of the scFv, the heavy chain variable domain is positioned at the C terminus of the light chain variable domain.

The multi-specific binding proteins described herein can further include one or more additional antigen-binding sites. The additional antigen-binding site(s) may be fused to the C-terminus of the constant region CH2 domain or to the C-terminus of the constant region CH3 domain, optionally via a linker sequence. In certain embodiments, the additional antigen-binding site(s) takes the form of a single-chain variable region (scFv) that is optionally disulfide-stabilized, resulting in a tetravalent or trivalent multi-specific binding protein. For example, a multi-specific binding protein includes an NKG2D-binding site, a HER2-binding site, a third antigen-binding site that binds a tumor-associated antigen, and an antibody constant region or a portion thereof sufficient to bind CD16, or a fourth antigen-binding site that binds CD16. Any one of these antigen-binding sites can either take the form of an Fab or an scFv, such as the scFv described above. In some embodiments, the third antigen-binding site binds a different tumor-associated antigen from HER2. In some embodiments, the third antigen-binding site binds to the same tumor-associated antigen HER2, and the exemplary formats are shown in FIGS. 2C and 2D. Accordingly, the multi-specific binding proteins can provide bivalent engagement of HER2. Bivalent engagement of HER2 by the multi-specific proteins can stabilize the HER2 on cancer cell surface, and enhance cytotoxicity of NK cells towards the cancer cells. Bivalent engagement of HER2 by the multi-specific proteins can confer stronger binding of the multi-specific proteins to the cancer cells, thereby facilitating stronger cytotoxic response of NK cells towards the cancer cells, especially towards cancer cells expressing a low level of HER2.

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793): 267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In some embodiments, the antibody constant domain comprises a CH2 domain and a CH3 domain of an IgG antibody, for example, a human IgG1 antibody. In some embodiments, mutations are introduced in the antibody constant domain to enable heterdimerization with another antibody constant domain. For example, if the antibody constant domain is derived from the constant domain of a human IgG1, the antibody constant domain can comprise an amino acid sequence at least 90% identical to amino acids 234-332 of a human IgG1 antibody, and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

In some embodiments, the antibody constant domain can comprise an amino acid sequence at least 90% identical to amino acids 234-332 of a human IgG1 antibody, and differs by one or more substitutions selected from the group consisting of Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E.

Individual components of the multi-specific binding proteins are described in more detail below.

### NKG2D-Binding Site

Upon binding to the NKG2D receptor and CD16 receptor on natural killer cells, and a tumor-associated antigen on cancer cells, the multi-specific binding proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys.

Table 1 lists peptide sequences of heavy chain variable domains and light chain variable domains that, in combination, can bind to NKG2D. In some embodiments, the heavy chain variable domain and the light chain variable domain are arranged in Fab format. In some embodiments, the heavy chain variable domain and the light chain variable domain are fused together to from an scFv.

The NKG2D binding domains listed in Table 1 can vary in their binding affinity to NKG2D, nevertheless, they all activate human NK cells.

Unless indicated otherwise, the CDR sequences provided in Table 1 are determined under Kabat.

TABLE 1

| | Heavy chain variable | Light chain variable |
|---|---|---|
| Clones | region amino acid sequence | region amino acid sequence |
| ADI- | QVQLQQWGAGLLKPSETLSLTCA | DIQMTQSPSTLSASVGDRVTI |
| 27705 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYNSYPITFGGGTKV |
| | (SEQ ID NO: 1) | EIK |
| | CDR1 (SEQ ID NO: 3) - | (SEQ ID NO: 2) |
| | GSFSGYYWS | |
| | CDR2 (SEQ ID NO: 4) - | |
| | EIDHSGSTNYNPSLKS | |
| | CDR3 (SEQ ID NO: 5) - | |
| | ARARGPWSFDP | |
| ADI- | QVQLQQWGAGLLKPSETLSLTCA | EIVLTQSPGTLSLSPGERATLS |
| 27724 | VYGGSFSGYYWSWIRQPPGKGLE | CRASQSVSSSYLAWYQQKPG |
| | WIGEIDHSGSTNYNPSLKSRVTISV | QAPRLLIYGASSRATGIPDRES |
| | DTSKNQFSLKLSSVTAADTAVYYC | GSGSGTDFTLTISRLEPEDFA |
| | ARARGPWSFDPWGQGTLVTVSS | VYYCQQYGSSPITFGGGTKV |
| | (SEQ ID NO: 6) | EIK |
| | | (SEQ ID NO: 7) |
| ADI- | QVQLQQWGAGLLKPSETLSLTCA | DIQMTQSPSTLSASVGDRVTI |
| 27740 | VYGGSFSGYYWSWIRQPPGKGLE | TCRASQSIGSWLAWYQQKPG |
| (A40) | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYHSFYTFGGGTKVE |
| | (SEQ ID NO: 8) | IK |
| | | (SEQ ID NO: 9) |
| ADI- | QVQLQQWGAGLLKPSETLSLTCA | DIQMTQSPSTLSASVGDRVTI |
| 27741 | VYGGSFSGYYWSWIRQPPGKGLE | TCRASQSIGSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQSNSYYTFGGGTKVE |
| | (SEQ ID NO: 10) | IK |
| | | (SEQ ID NO: 11) |
| ADI- | QVQLQQWGAGLLKPSETLSLTCA | DIQMTQSPSTLSASVGDRVTI |
| 27743 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYNSYPTFGGGTKVE |
| | (SEQ ID NO: 12) | IK |
| | | (SEQ ID NO: 13) |
| ADI- | QVQLQQWGAGLLKPSETLSLTCA | ELQMTQSPSSLSASVGDRVTI |
| 28153 | VYGGSFSGYYWSWIRQPPGKGLE | TCRTSQSISSYLNWYQQKPG |
| | WIGEIDHSGSTNYNPSLKSRVTISV | QPPKLLIYWASTRESGVPDRF |
| | DTSKNQFSLKLSSVTAADTAVYYC | SGSGSGTDFTLTISSLQPEDSA |
| | ARARGPWGFDPWGQGTLVTVSS | TYYCQQSYDIPYTFGQGTKL |
| | (SEQ ID NO: 14) | EIK |
| | | (SEQ ID NO: 15) |
| ADI- | QVQLQQWGAGLLKPSETLSLTCA | DIQMTQSPSTLSASVGDRVTI |
| 28226 | VYGGSFSGYYWSWIRQPPGKGLE | TCRASQSISSWLAWYQQKPG |
| (C26) | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYGSFPITFGGGTKVE |
| | (SEQ ID NO: 16) | IK |
| | | (SEQ ID NO: 17) |
| ADI- | QVQLQQWGAGLLKPSETLSLTCA | DIQMTQSPSTLSASVGDRVTI |
| 28154 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTDFTLTISSLQPDDFA |
| | ARARGPWSFDPWGQGTLVTVSS | TYYCQQSKEVPWTFGQGTK |
| | (SEQ ID NO: 18) | VEIK |
| | | (SEQ ID NO: 19) |
| ADI- | QVQLQQWGAGLLKPSETLSLTCA | DIQMTQSPSTLSASVGDRVTI |
| 29399 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYNSFPTFGGGTKVEI |
| | (SEQ ID NO: 20) | K |
| | | (SEQ ID NO: 21) |
| ADI- | QVQLQQWGAGLLKPSETLSLTCA | DIQMTQSPSTLSASVGDRVTI |
| 29401 | VYGGSFSGYYWSWIRQPPGKGLE | TCRASQSIGSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYDIYPTFGGGTKVEI |
| | (SEQ ID NO: 22) | K |
| | | (SEQ ID NO: 23) |
| ADI- | QVQLQQWGAGLLKPSETLSLTCA | DIQMTQSPSTLSASVGDRVTI |
| 29403 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYDSYPTFGGGTKVE |
| | (SEQ ID NO: 24) | IK |
| | | (SEQ ID NO: 25) |
| ADI- | QVQLQQWGAGLLKPSETLSLTCA | DIQMTQSPSTLSASVGDRVTI |
| 29405 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYGSFPTFGGGTKVEI |
| | (SEQ ID NO: 26) | K |
| | | (SEQ ID NO: 27) |
| ADI- | QVQLQQWGAGLLKPSETLSLTCA | DIQMTQSPSTLSASVGDRVTI |
| 29407 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYQSFPTFGGGTKVEI |
| | (SEQ ID NO: 28) | K |
| | | (SEQ ID NO: 29) |
| ADI- | QVQLQQWGAGLLKPSETLSLTCA | DIQMTQSPSTLSASVGDRVTI |
| 29419 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYSSFSTFGGGTKVEI |
| | (SEQ ID NO: 30) | K |
| | | (SEQ ID NO: 31) |
| ADI- | QVQLQQWGAGLLKPSETLSLTCA | DIQMTQSPSTLSASVGDRVTI |
| 29421 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYESYSTFGGGTKVE |
| | (SEQ ID NO: 32) | IK |
| | | (SEQ ID NO: 33) |
| ADI- | QVQLQQWGAGLLKPSETLSLTCA | DIQMTQSPSTLSASVGDRVTI |
| 29424 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYDSFITFGGGTKVEI |
| | (SEQ ID NO: 34) | K |
| | | (SEQ ID NO: 35) |
| ADI- | QVQLQQWGAGLLKPSETLSLTCA | DIQMTQSPSTLSASVGDRVTI |
| 29425 | VYGGSFSGYYWSWIRQPPGKGLE | TCRASQSISSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYQSYPTFGGGTKVE |
| | (SEQ ID NO: 36) | IK |
| | | (SEQ ID NO: 37) |
| ADI- | QVQLQQWGAGLLKPSETLSLTCA | DIQMTQSPSTLSASVGDRVTI |
| 29426 | VYGGSFSGYYWSWIRQPPGKGLE | TCRASQSIGSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYHSFPTFGGGTKVEI |
| | (SEQ ID NO: 38) | K |
| | | (SEQ ID NO: 39) |
| ADI- | QVQLQQWGAGLLKPSETLSLTCA | DIQMTQSPSTLSASVGDRVTI |
| 29429 | VYGGSFSGYYWSWIRQPPGKGLE | TCRASQSIGSWLAWYQQKPG |
| | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYELYSYTFGGGTKV |
| | (SEQ ID NO: 40) | EIK |
| | | (SEQ ID NO: 41) |
| ADI- | QVQLQQWGAGLLKPSETLSLTCA | DIQMTQSPSTLSASVGDRVTI |
| 29447 | VYGGSFSGYYWSWIRQPPGKGLE | TCRASQSISSWLAWYQQKPG |
| (F47) | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCQQYDTFITFGGGTKVEI |
| | (SEQ ID NO: 42) | K |
| | | (SEQ ID NO: 43) |
| ADI- | QVQLVQSGAEVKKPGSSVKVSCK | DIVMTQSPDSLAVSLGERATI |
| 27727 | ASGGTFSSYAISWVRQAPGQGLE | NCKSSQSVLYSSNNKNYLAW |
| | WMGGIIPIFGTANYAQKFQGRVTI | YQQKPGQPPKLLIYWASTRE |
| | TADESTSTAYMELSSLRSEDTAVY | SGVPDRFSGSGSGTDFTLTISS |
| | YCARGDSSIRHAYYYYGMDVWG | LQAEDVAVYYCQQYYSTPIT |
| | QGTTVTVSS | FGGGTKVEIK |
| | (SEQ ID NO: 44) | (SEQ ID NO: 48) |
| | CDR1 (SEQ ID NO: 45) - | CDR1 (SEQ ID NO: 49) - |
| | GTFSSYAIS (non-Kabat) or SYAIS | KSSQSVLYSSNNKNYLA |
| | (SEQ ID NO: 158) | CDR2 (SEQ ID NO: 50) - |
| | CDR2 (SEQ ID NO: 46) - | WASTRES |
| | GIIPIFGTANYAQKFQG | CDR3 (SEQ ID NO: 51) - |
| | CDR3 (SEQ ID NO: 47) - | QQYYSTPIT |
| | ARGDSSIRHAYYYYGMDV (non- | |
| | Kabat) or GDSSIRHAYYYYGMDV | |
| | (SEQ ID NO: 159) | |
| ADI- | QLQLQESGPGLVKPSETLSLTCTVS | EIVLTQSPATLSLSPGERATLS |
| 29443 | GGSISSSSYYWGWIRQPPGKGLEW | CRASQSVSRYLAWYQQKPG |
| (F43) | IGSIYYSGSTYYNPSLKSRVTISVDT | QAPRLLIYDASNRATGIPARF |
| | SKNQFSLKLSSVTAADTAVYYCAR | SGSGSGTDFTLTISSLEPEDFA |
| | GSDRFHPYFDYWGQGTLVTVSS | VYYCQQFDTWPPTFGGGTKV |
| | (SEQ ID NO: 52) | EIK |
| | CDR1 (SEQ ID NO: 53) - | (SEQ ID NO: 56) |
| | GSISSSSYYWG (non-Kabat) or | CDR1 (SEQ ID NO: 57) - |
| | SSSYYWG (SEQ ID NO: 160) CDR2 | RASQSVSRYLA |
| | (SEQ ID NO: 54) - | CDR2 (SEQ ID NO: 58) - |
| | SIYYSGSTYYNPSLKS | DASNRAT |
| | CDR3 (SEQ ID NO: 55) - | CDR3 (SEQ ID NO: 59) - |
| | ARGSDRFHPYFDY (non-Kabat) or | QQFDTWPPT |
| | GSDRFHPYFDY (SEQ ID NO: 161) | |
| ADI- | QVQLQQWGAGLLKPSETLSLTCA | DIQMTQSPSTLSASVGDRVTI |
| 29404 | VYGGSFSGYYWSWIRQPPGKGLE | TCRASQSISSWLAWYQQKPG |
| (F04) | WIGEIDHSGSTNYNPSLKSRVTISV | KAPKLLIYKASSLESGVPSRF |
| | DTSKNQFSLKLSSVTAADTAVYYC | SGSGSGTEFTLTISSLQPDDFA |
| | ARARGPWSFDPWGQGTLVTVSS | TYYCEQYDSYPTFGGGTKVE |
| | (SEQ ID NO: 60) | IK |
| | | (SEQ ID NO: 61) |
| ADI- | QVQLVQSGAEVKKPGSSVKVSCK | DIVMTQSPDSLAVSLGERATI |
| 28200 | ASGGTESSYAISWVRQAPGQGLE | NCESSQSLLNSGNQKNYLTW |
| | WMGGIIPIFGTANYAQKFQGRVTI | YQQKPGQPPKPLIYWASTRES |
| | TADESTSTAYMELSSLRSEDTAVY | GVPDRFSGSGSGTDFTLTISSL |
| | YCARRGRKASGSFYYYYGMDVW | QAEDVAVYYCONDYSYPYT |
| | GQGTTVTVSS | FGQGTKLEIK |
| | (SEQ ID NO: 62) | (SEQ ID NO: 66) |
| | CDR1 (SEQ ID NO: 63) - | CDR1 (SEQ ID NO: 67) - |
| | GTFSSYAIS (non-Kabat) or SYAIS | ESSQSLLNSGNQKNYLT |
| | (SEQ ID NO: 158) CDR2 (SEQ ID | CDR2 (SEQ ID NO: 68) - |
| | NO: 64) - GIIPIFGTANYAQKFQG | WASTRES |
| | CDR3 (SEQ ID NO: 65) - | CDR3 (SEQ ID NO: 69) - |
| | ARRGRKASGSFYYYYGMDV | QNDYSYPYT |
| ADI- | QVQLVQSGAEVKKPGASVKVSCK | EIVMTQSPATLSVSPGERATL |
| 29379 | ASGYTFTSYYMHWVRQAPGQGLE | SCRASQSVSSNLAWYQQKPG |
| (E79) | WMGIINPSGGSTSYAQKFQGRVT | |
| | MTRDTSTSTVYMELSSLRSEDTAV | QAPRLLIYGASTRATGIPARF |
| | YYCARGAPNYGDTTHDYYYMDV | SGSGSGTEFTLTISSLQSEDFA |
| | WGKGTTVTVSS | VYYCQQYDDWPFTFGGGTK |
| | (SEQ ID NO: 70) | VEIK |
| | CDR1 (SEQ ID NO: 71) - | (SEQ ID NO: 74) |
| | YTFTSYYMH (non-Kabat) or | CDR1 (SEQ ID NO: 75) - |
| | SYYMH (SEQ ID NO: 162) | RASQSVSSNLA |
| | CDR2 (SEQ ID NO: 72) - | CDR2 (SEQ ID NO: 76) - |
| | IINPSGGSTSYAQKFQG | GASTRAT |
| | CDR3 (SEQ ID NO: 73) - | CDR3 (SEQ ID NO: 77) - |
| | ARGAPNYGDTTHDYYYMDV (non- | QQYDDWPFT |
| | Kabat) or | |
| | GAPNYGDTTHDYYYMDV (SEQ ID | |
| | NO: 163) | |
| ADI- | QVQLVQSGAEVKKPGASVKVSCK | EIVLTQSPGTLSLSPGERATLS |
| 29463 | ASGYTFTGYYMHWVRQAPGQGL | CRASQSVSSNLAWYQQKPGQ |
| (F63) | EWMGWINPNSGGTNYAQKFQGR | APRLLIYGASTRATGIPARFS |
| | VTMTRDTSISTAYMELSRLRSDDT | GSGSGTEFTLTISSLQSEDFAV |
| | AVYYCARDTGEYYDTDDHGMDV | YYCQQDDYWPPTFGGGTKV |
| | WGQGTTVTVSS | EIK |
| | (SEQ ID NO: 78) | (SEQ ID NO: 82) |
| | CDR1 (SEQ ID NO: 79) - | CDR1 (SEQ ID NO: 75) - |
| | YTFTGYYMH (non-Kabat) or | RASQSVSSNLA |
| | GYYMH (SEQ ID NO: 164) | CDR2 (SEQ ID NO: 76) - |
| | CDR2 (SEQ ID NO: 80) - | GASTRAT |
| | WINPNSGGTNYAQKFQG | CDR3 (SEQ ID NO: 85) - |
| | CDR3 (SEQ ID NO: 81) - | QQDDYWPPT |
| | ARDTGEYYDTDDHGMDV (non- | |
| | Kabat) or DTGEYYDTDDHGMDV | |
| | (SEQ ID NO: 165) | |
| ADI- | EVQLLESGGGLVQPGGSLRLSCAA | DIQMTQSPSSVSASVGDRVTI |
| 27744 | SGFTFSSYAMSWVRQAPGKGLEW | TCRASQGIDSWLAWYQQKP |
| (A44) | VSAISGSGGSTYYADSVKGRFTISR | GKAPKLLIYAASSLQSGVPSR |
| | DNSKNTLYLQMNSLRAEDTAVYY | FSGSGSGTDFTLTISSLQPEDF |
| | CAKDGGYYDSGAGDYWGQGTLV | ATYYCQQGVSYPRTFGGGTK |
| | TVSS | VEIK |
| | (SEQ ID NO: 86) | (SEQ ID NO: 90) |
| | CDR1 (SEQ ID NO: 87) - | CDR1 (SEQ ID NO: 91) - |
| | FTFSSYAMS (non-Kabat) or SYAMS | RASQGIDSWLA |
| | (SEQ ID NO: 166) | CDR2 (SEQ ID NO: 92) - |
| | CDR2 (SEQ ID NO: 88) - | AASSLQS |
| | AISGSGGSTYYADSVKG | CDR3 (SEQ ID NO: 93) - |
| | CDR3 (SEQ ID NO: 89) - | QQGVSYPRT |
| | AKDGGYYDSGAGDY (non-Kabat) | |
| | or DGGYYDSGAGDY (SEQ ID | |
| | NO: 167) | |
| ADI- | EVQLVESGGGLVKPGGSLRLSCAA | DIQMTQSPSSVSASVGDRVTI |
| 27749 | SGFTFSSYSMNWVRQAPGKGLEW | TCRASQGISSWLAWYQQKPG |
| (A49) | VSSISSSSSYIYYADSVKGRFTISRD | KAPKLLIYAASSLQSGVPSRF |
| | NAKNSLYLQMNSLRAEDTAVYYC | SGSGSGTDFTLTISSLOPEDFA |
| | ARGAPMGAAAGWFDPWGQGTLV | TYYCQQGVSFPRTFGGGTKV |
| | TVSS | EIK |
| | (SEQ ID NO: 94) | (SEQ ID NO: 98) |
| | CDR1 (SEQ ID NO: 95) - | CDR1 (SEQ ID NO: 99) - |
| | FTFSSYSMN (non-Kabat) or SYSMN | RASQGISSWLA |
| | (SEQ ID NO: 168) | CDR2 (SEQ ID NO: 100) - |
| | CDR2 (SEQ ID NO: 96) - | AASSLQS |
| | SISSSSSYIYYADSVKG | CDR3 (SEQ ID NO: 101) - |
| | CDR3 (SEQ ID NO: 97) - | QQGVSFPRT |
| | ARGAPMGAAAGWFDP (non-Kabat) | |
| | or GAPMGAAAGWFDP (SEQ ID | |
| | NO: 169) | |
| ADI- | QVQLVQSGAEVKKPGASVKVSCK | EIVLTQSPATLSLSPGERATLS |
| 29378 | ASGYTFTSYYMHWVRQAPGQGLE | CRASQSVSSYLAWYQQKPGQ |
| (E78) | WMGIINPSGGSTSYAQKFQGRVT | APRLLIYDASNRATGIPARFS |
| | MTRDTSTSTVYMELSSLRSEDTAV | GSGSGTDFTLTISSLEPEDFAV |
| | YYCAREGAGFAYGMDYYYMDV | YYCQQSDNWPFTFGGGTKVE |
| | WGKGTTVTVSS | IK |
| | (SEQ ID NO: 102) | (SEQ ID NO: 106) |
| | CDR1 (SEQ ID NO: 71) - | CDR1 (SEQ ID NO: 107) - |
| | YTFTSYYMH (non-Kabat) or | RASQSVSSYLA |
| | SYYMH (SEQ ID NO: 162) | CDR2 (SEQ ID NO: 108) - |
| | CDR2 (SEQ ID NO: 72) - | DASNRAT |
| | IINPSGGSTSYAQKFQG | CDR3 (SEQ ID NO: 109) - |
| | CDR3 (SEQ ID NO: 105) - | QQSDNWPFT |
| | AREGAGFAYGMDYYYMDV (non- | |
| | Kabat) or EGAGFAYGMDYYYMDV | |
| | (SEQ ID NO: 170) | |
| A49MI | EVQLVESGGGLVKPGGSLRLSCAA | DIQMTQSPSSVSASVGDRVTI |
| | SGFTFSSYSMNWVRQAPGKGLEW | TCRASQGISSWLAWYQQKPG |
| | VSSISSSSSYIYYADSVKGRFTISRD | KAPKLLIYAASSLQSGVPSRF |
| | NAKNSLYLQMNSLRAEDTAVYYC | SGSGSGTDFTLTISSLQPEDFA |
| | ARGAPIGAAAGWFDPWGQGTLVT | TYYCQQGVSFPRTFGGGTKV |
| | VSS | EIK |
| | (SEQ ID NO: 144) | (SEQ ID NO: 98) |
| | CDR1 (SEQ ID NO: 95) - | CDR1 (SEQ ID NO: 99) - |
| | FTFSSYSMN (non-Kabat) or SYSMN | RASQGISSWLA |
| | (SEQ ID NO: 168) | CDR2 (SEQ ID NO: 100) - |
| | CDR2 (SEQ ID NO: 96) - | AASSLQS |
| | SISSSSSYIYYADSVKG | CDR3 (SEQ ID NO: 101) - |
| | CDR3: (non-Kabat) | QQGVSFPRT |
| | ARGAPIGAAAGWFDP (SEQ ID | |
| | NO: 172) or GAPIGAAAGWFDP | |
| | (SEQ ID NO: 173) | |
| A49MQ | EVQLVESGGGLVKPGGSLRLSCAA | DIQMTQSPSSVSASVGDRVTI |
| | SGFTFSSYSMNWVRQAPGKGLEW | TCRASQGISSWLAWYQQKPG |
| | VSSISSSSSYIYYADSVKGRFTISRD | KAPKLLIYAASSLQSGVPSRF |
| | NAKNSLYLQMNSLRAEDTAVYYC | SGSGSGTDFTLTISSLQPEDFA |
| | ARGAPQGAAAGWFDPWGQGTLV | TYYCQQGVSFPRTFGGGTKV |
| | TVSS | EIK |
| | (SEQ ID NO: 174) | (SEQ ID NO: 98) |
| | CDR1 (SEQ ID NO: 95) - | CDR1 (SEQ ID NO: 99) - |
| | FTFSSYSMN (non-Kabat) or SYSMN | RASQGISSWLA |
| | (SEQ ID NO: 168) | CDR2 (SEQ ID NO: 100) - |
| | CDR2 (SEQ ID NO: 96) - | AASSLQS |
| | SISSSSSYIYYADSVKG | CDR3 (SEQ ID NO: 101) - |
| | CDR3 (non-Kabat) (SEQ ID NO: 175) - | QQGVSFPRT |
| | ARGAPQGAAAGWFDP or CDR3 | |
| | (SEQ ID NO: 176) - | |
| | GAPQGAAAGWFDP | |
| A49ML | EVQLVESGGGLVKPGGSLRLSCAA | DIQMTQSPSSVSASVGDRVTI |
| | SGFTFSSYSMNWVRQAPGKGLEW | TCRASQGISSWLAWYQQKPG |
| | VSSISSSSSYIYYADSVKGRFTISRD | KAPKLLIYAASSLQSGVPSRF |
| | NAKNSLYLQMNSLRAEDTAVYYC | SGSGSGTDFTLTISSLQPEDFA |
| | ARGAPLGAAAGWFDPWGQGTLV | TYYCQQGVSFPRTFGGGTKV |
| | TVSS | EIK |
| | (SEQ ID NO: 177) | (SEQ ID NO: 98) |
| | CDR1 (SEQ ID NO: 95) - | CDR1 (SEQ ID NO: 99) - |
| | FTFSSYSMN (non-Kabat) or SYSMN | RASQGISSWLA |
| | (SEQ ID NO: 168) | CDR2 (SEQ ID NO: 100) - |
| | CDR2 (SEQ ID NO: 96) - | AASSLQS |
| | SISSSSSYIYYADSVKG | CDR3 (SEQ ID NO: 101) - |
| | CDR3 (non-Kabat) (SEQ ID NO: 178) - | QQGVSFPRT |
| | ARGAPLGAAAGWFDP or CDR3 | |
| | (SEQ ID NO: 179) - | |
| | GAPLGAAAGWFDP | |
| A49MF | EVQLVESGGGLVKPGGSLRLSCAA | DIQMTQSPSSVSASVGDRVTI |
| | SGFTFSSYSMNWVRQAPGKGLEW | TCRASQGISSWLAWYQQKPG |
| | VSSISSSSSYIYYADSVKGRFTISRD | KAPKLLIYAASSLQSGVPSRF |
| | NAKNSLYLQMNSLRAEDTAVYYC | SGSGSGTDFTLTISSLQPEDFA |
| | ARGAPFGAAAGWFDPWGQGTLV | TYYCQQGVSFPRTFGGGTKV |
| | TVSS | EIK |
| | (SEQ ID NO: 180) | (SEQ ID NO: 98) |
| | CDR1 (SEQ ID NO: 95) - | CDR1 (SEQ ID NO: 99) - |
| | FTFSSYSMN (non-Kabat) or SYSMN | RASQGISSWLA |
| | (SEQ ID NO: 168) | CDR2 (SEQ ID NO: 100) - |
| | CDR2 (SEQ ID NO: 96) - | AASSLQS CDR3 (SEQ ID NO: 101) - |
| | SISSSSSYIYYADSVKG | QQGVSFPRT |
| | CDR3 (non-Kabat) (SEQ ID NO: 181) - | |
| | ARGAPFGAAAGWFDP or CDR3 | |
| | (SEQ ID NO: 182) - | |
| | GAPFGAAAGWFDP | |
| A49MV | EVQLVESGGGLVKPGGSLRLSCAA | DIQMTQSPSSVSASVGDRVTI |
| | SGFTFSSYSMNWVRQAPGKGLEW | TCRASQGISSWLAWYQQKPG |
| | VSSISSSSSYIYYADSVKGRFTISRD | KAPKLLIYAASSLQSGVPSRF |
| | NAKNSLYLQMNSLRAEDTAVYYC | SGSGSGTDFTLTISSLQPEDFA |
| | ARGAPVGAAAGWFDPWGQGTLV | TYYCQQGVSFPRTFGGGTKV |
| | TVSS | EIK |
| | (SEQ ID NO: 183) | (SEQ ID NO: 98) |
| | CDR1 (SEQ ID NO: 95) - | CDR1 (SEQ ID NO: 99) - |
| | | RASQGISSWLA |
| | FTFSSYSMN (non-Kabat) or SYSMN | CDR2 (SEQ ID NO: 100) - |
| | (SEQ ID NO: 168) | AASSLQS |
| | CDR2 (SEQ ID NO: 96) - | CDR3 (SEQ ID NO: 101) - |
| | SISSSSSYIYYADSVKG | QQGVSFPRT |
| | CDR3 (non-Kabat) (SEQ ID NO: 184) - | |
| | ARGAPVGAAAGWFDP or CDR3 | |
| | (SEQ ID NO: 185) - | |
| | GAPVGAAAGWFDP | |
| A49- | EVQLVESGGGLVKPGGSLRLSCAA | DIQMTQSPSSVSASVGDRVTI |
| consensus | SGFTFSSYSMNWVRQAPGKGLEW | TCRASQGISSWLAWYQQKPG |
| | VSSISSSSSYIYYADSVKGRFTISRD | KAPKLLIYAASSLQSGVPSRF |
| | NAKNSLYLQMNSLRAEDTAVYYC | SGSGSGTDFTLTISSLQPEDFA |
| | ARGAPXGAAAGWFDPWGQGTLV | TYYCQQGVSFPRTFGGGTKV |
| | TVSS, wherein X is M, L, I, V, | EIK |
| | Q, or F | (SEQ ID NO: 98) |
| | (SEQ ID NO: 186) | CDR1 (SEQ ID NO: 99) - |
| | CDR1 (SEQ ID NO: 95) - | RASQGISSWLA |
| | FTFSSYSMN (non-Kabat) or SYSMN | CDR2 (SEQ ID NO: 100) - |
| | (SEQ ID NO: 168) | AASSLQS |
| | CDR2 (SEQ ID NO: 96) - | CDR3 (SEQ ID NO: 101) - |
| | SISSSSSYIYYADSVKG | QQGVSFPRT |
| | CDR3 (non-Kabat) (SEQ ID NO: 187) - | |
| | ARGAPXGAAAGWFDP or CDR3 | |
| | (SEQ ID NO: 188) - | |
| | GAPXGAAAGWFDP, wherein X is | |
| | M, L, I, V, Q, or F | |

Alternatively, a heavy chain variable domain represented by SEQ ID NO:110 can be paired with a light chain variable domain represented by SEQ ID NO:111 to form an antigen-binding site that can bind to NKG2D, as illustrated in U.S. Pat. No. 9,273,136.

SEQ ID NO: 110
QVQLVESGGGLVKPGGSLRLSCAASGFTFSSYGMHWVRQAPGKGLEWVA
FIRYDGSNKYYADSVKGRFTISRDNSKNTLYLQMNSLRAEDTAVYYCAK
DRGLGDGTYFDYWGQGTTVTVSS
SEQ ID NO: 111
QSALTQPASVSGSPGQSITISCSGSSSNIGNNAVNWYQQLPGKAPKLLI
YYDDLLPSGVSDRFSGSKSGTSAFLAISGLQSEDEADYYCAAWDDSLNG
PVFGGGTKLTVL

Alternatively, a heavy chain variable domain represented by SEQ ID NO:112 can be paired with a light chain variable domain represented by SEQ ID NO:113 to form an antigen-binding site that can bind to NKG2D, as illustrated in U.S. Pat. No. 7,879,985.

SEQ ID NO: 112
QVHLQESGPGLVKPSETLSLTCTVSDDSISSYYWSWIRQPPGKGLEWIG
HISYSGSANYNPSLKSRVTISVDTSKNQFSLKLSSVTAADTAVYYCANW
DDAFNIWGQGTMVTVSS
SEQ ID NO: 113
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPWT
FGQGTKVEIK

The multi-specific binding proteins can bind to NKG2D-expressing cells, which include but are not limited to NK cells, γδ T cells and CD8+αβ T cells. Upon NKG2D binding, the multi-specific binding proteins may block natural ligands, such as ULBP6 and MICA, from binding to NKG2D and activating NK cells.

The multi-specific binding proteins binds to cells expressing CD16, an Fc receptor on the surface of leukocytes including natural killer cells, macrophages, neutrophils, eosinophils, mast cells, and follicular dendritic cells. A protein of the present disclosure binds to NKG2D with an affinity of K_{D} of 2 nM to 120 nM, e.g., 2 nM to 110 nM, 2 nM to 100 nM, 2 nM to 90 nM, 2 nM to 80 nM, 2 nM to 70 nM, 2 nM to 60 nM, 2 nM to 50 nM, 2 nM to 40 nM, 2 nM to 30 nM, 2 nM to 20 nM, 2 nM to 10 nM, about 15 nM, about 14 nM, about 13 nM, about 12 nM, about 11 nM, about 10 nM, about 9 nM, about 8 nM, about 7 nM, about 6 nM, about 5 nM, about 4.5 nM, about 4 nM, about 3.5 nM, about 3 nM, about 2.5 nM, about 2 nM, about 1.5 nM, about 1 nM, between about 0.5 nM to about 1 nM, about 1 nM to about 2 nM, about 2 nM to 3 nM, about 3 nM to 4 nM, about 4 nM to about 5 nM, about 5 nM to about 6 nM, about 6 nM to about 7 nM, about 7 nM to about 8 nM, about 8 nM to about 9 nM, about 9 nM to about 10 nM, about 1 nM to about 10 nM, about 2 nM to about 10 nM, about 3 nM to about 10 nM, about 4 nM to about 10 nM, about 5 nM to about 10 nM, about 6 nM to about 10 nM, about 7 nM to about 10 nM, or about 8 nM to about 10 nM. In some embodiments, NKG2D-binding sites bind to NKG2D with a K_{D} of 10 to 62 nM.

### HER2-Binding Site

The HER2-binding site of the multi-specific binding protein disclosed herein comprises a heavy chain variable domain and a light chain variable domain fused together to from an scFv.

Table 2 lists peptide sequences of heavy chain variable domains and light chain variable domains that, in combination, can bind to HER2.

TABLE 2

| | Heavy chain variable | Light chain variable |
|---|---|---|
| Clones | domain amino acid sequence | domain amino acid sequence |
| Trastuzumab | EVQLVESGGGLVQPGGSLRLSCA | DIQMTQSPSSLSASVGDRVTITCR |
| | ASGFNIKDTYIHWVRQAPGKGLE | ASQDVNTAVAWYQQKPGKAPK |
| | WVARIYPTNGYTRYADSVKGRF | LLIYSASFLYSGVPSRFSGSRSGT |
| | TISADTSKNTAYLQMNSLRAEDT | DFTLTISSLQPEDFATYYCQQHY |
| | AVYYCSRWGGDGFYAMDYWG | TTPPTFGQGTKVEIK |
| | QGTLVTVSS | (SEQ ID NO: 118) |
| | (SEQ ID NO: 114) | CDR1(SEQ ID NO: 119) - |
| | CDR1(SEQ ID NO: 115) - GFNIKDT | QDVNTAVA |
| | CDR2 (SEQ ID NO: 116) - YPTNGY | CDR2 (SEQ ID NO: 120) - |
| | CDR3 (SEQ ID NO: 117) - | SASFLYS |
| | WGGDGFYAMDY | CDR3 (SEQ ID NO: 121) - |
| | | QQHYTTPPT |
| Trastuzumab | EVQLVESGGGLVQPGGSLRLSCA | DIQMTQSPSSLSASVGDRVTITCR |
| (VH and VL | ASGFNIKDTYIHWVRQAPGKCLE | ASQDVNTAVAWYQQKPGKAPK |
| in scFv | WVARIYPTNGYTRYADSVKGRF | LLIYSASFLYSGVPSRFSGSRSGT |
| construct) | TISADTSKNTAYLQMNSLRAEDT | DFTLTISSLQPEDFATYYCQQHY |
| | AVYYCSRWGGDGFYAMDYWG | TTPPTFGCGTKVEIK |
| | QGTLVTVSS (SEQ ID NO: 195) | (SEQ ID NO: 196) |
| | CDR1 (SEQ ID NO: 115) - GFNIKDT | CDR1(SEQ ID NO: 119) - |
| | CDR2 (SEQ ID NO: 116) - YPTNGY | QDVNTAVA |
| | CDR3 (SEQ ID NO: 117) - | CDR2 (SEQ ID NO: 120) - |
| | WGGDGFYAMDY | SASFLYS |
| | | CDR3 (SEQ ID NO: 121) - |
| | | QQHYTTPPT |
| Trastuzumab- | DIQMTQSPSSLSASVGDRVTITCRASQDVNTAVAWYQQKPGKAPKLL | |
| scFv | IYSASFLYSGVPSRFSGSRSGTDFTLTISSLQPEDFATYYCQQHYTTPPT | |
| | FGCGTKVEIKGGGGSGGGGSGGGGSGGGGSEVQLVESGGGLVQPGG | |
| | SLRLSCAASGFNIKDTYIHWVRQAPGKCLEWVARIYPTNGYTRYADS | |
| | VKGRFTISADTSKNTAYLQMNSLRAEDTAVYYCSRWGGDGFYAMD | |
| | YWGQGTLVTVSS (SEQ ID NO: 139) | |
| Pertuzumab | EVQLVESGGGLVQPGGSLRLSCA | DIQMTQSPSSLSASVGDRVTITCK |
| | ASGFTFTDYTMDWVRQAPGKGL | ASQDVSIGVAWYQQKPGKAPKL |
| | EWVADVNPNSGGSIYNQRFKGR | LIYSASYRYTGVPSRFSGSGSGTD |
| | FTLSVDRSKNTLYLQMNSLRAED | FTLTISSLQPEDFATYYCQQYYIY |
| | TAVYYCARNLGPSFYFDYWGQG | PYTFGQGTKVEIKR |
| | TLVTVSSA | (SEQ ID NO: 126) |
| | (SEQ ID NO: 122) | CDR1 (SEQ ID NO: 127) - |
| | CDR1 (SEQ ID NO: 123) - | QDVSIGVA |
| | GFTFTDY | CDR2 (SEQ ID NO: 128) - |
| | CDR2 (SEQ ID NO: 124) - NPNSGG | SASYRYT |
| | CDR3 (SEQ ID NO: 125) - | CDR3 (SEQ ID NO: 129) - |
| | NLGPSFYFDY | QQYYIYPYT |
| Pertuzumab | EVQLVESGGGLVQPGGSLRLSCA | DIQMTQSPSSLSASVGDRVTITCK |
| (VH and VL | ASGFTFTDYTMDWVRQAPGKCL | ASQDVSIGVAWYQQKPGKAPKL |
| in scFv | EWVADVNPNSGGSIYNQRFKGR | LIYSASYRYTGVPSRFSGSGSGTD |
| construct) | FTLSVDRSKNTLYLQMNSLRAED | FTLTISSLQPEDFATYYCQQYYIY |
| | TAVYYCARNLGPSFYFDYWGQG | PYTFGCGTKVEIKR |
| | TLVTVSSA (SEQ ID NO: 197) | (SEQ ID NO: 198) |
| | CDR1 (SEQ ID NO: 123) - | CDR1 (SEQ ID NO: 127) - |
| | GFTFTDY | QDVSIGVA |
| | CDR2 (SEQ ID NO: 124) - NPNSGG | CDR2 (SEQ ID NO: 128) - |
| | CDR3 (SEQ ID NO: 125) - | SASYRYT |
| | NLGPSFYFDY | CDR3 (SEQ ID NO: 129) - |
| | | QQYYIYPYT |
| Pertuzumab | DIQMTQSPSSLSASVGDRVTITCKASQDVSIGVAWYQQKPGKAPKLLI | |
| scFv | YSASYRYTGVPSRFSGSGSGTDFTLTISSLQPEDFATYYCQQYYIYPYT | |
| | FGCGTKVEIKRGGGGSGGGGSGGGGSGGGGSEVQLVESGGGLVQPG | |
| | GSLRLSCAASGFTFTDYTMDWVRQAPGKCLEWVADVNPNSGGSIYN | |
| | QRFKGRFTLSVDRSKNTLYLQMNSLRAEDTAVYYCARNLGPSFYFDY | |
| | WGQGTLVTVSSA (SEQ ID NO: 189) | |
| MGAH22 | QVQLQQSGPELVKPGASLKLSCT | DIVMTQSHKFMSTSVGDRVSITC |
| (US | ASGFNIKDTYIHWVKQRPEQGLE | KASQDVNTAVAWYQQKPGHSP |
| 8,802,093) | WIGRIYPTNGYTRYDPKFQDKAT | KLLIYSASFRYTGVPDRFTGSRSG |
| | ITADTSSNTAYLQVSRLTSEDTA | TDFTFTISSVQAEDLAVYYCQQH |
| | VYYCSRWGGDGFYAMDYWGQ | YTTPPTFGGGTKVEIK |
| | GASVTVSS | (SEQ ID NO: 134) |
| | (SEQ ID NO: 130) | CDR1 (SEQ ID NO: 135) - |
| | CDR1 (SEQ ID NO: 131) - | QDVNTAVA |
| | GFNIKDT | CDR2 (SEQ ID NO: 136) - |
| | CDR2 (SEQ ID NO: 132) - YPTNGY | SASFRYT |
| | CDR3 (SEQ ID NO: 133) - | CDR3 (SEQ ID NO: 137) - |
| | WGGDGFYAMDY | QQHYTTPPT |
| MGAH22 | QVQLQQSGPELVKPGASLKLSCT | DIVMTQSHKFMSTSVGDRVSITC |
| (VH and VL | ASGFNIKDTYIHWVKQRPEQCLE | KASQDVNTAVAWYQQKPGHSP |
| in scFv | WIGRIYPTNGYTRYDPKFQDKAT | KLLIYSASFRYTGVPDRFTGSRSG |
| construct) | ITADTSSNTAYLQVSRLTSEDTA | TDFTFTISSVQAEDLAVYYCQQH |
| | VYYCSRWGGDGFYAMDYWGQ | YTTPPTFGCGTKVEIKR (SEQ ID |
| | GASVTVSSA (SEQ ID NO: 199) | NO: 200) |
| | CDR1 (SEQ ID NO: 131) - | CDR1 (SEQ ID NO: 135) - |
| | GFNIKDT | QDVNTAVA |
| | CDR2 (SEQ ID NO: 132) - YPTNGY | CDR2 (SEQ ID NO: 136) - |
| | CDR3 (SEQ ID NO: 133) - | SASFRYT |
| | WGGDGFYAMDY | CDR3 (SEQ ID NO: 137) - |
| | | QQHYTTPPT |

| | |
|---|---|
| MGAH22 | DIVMTQSHKFMSTSVGDRVSITCKASQDVNTAVAWYQQKPGHSPKL |
| scFv | LIYSASFRYTGVPDRFTGSRSGTDFTFTISSVQAEDLAVYYCQQHYTTP |
| | PTFGCGTKVEIKRGGGGSGGGGSGGGGSGGGGSQVQLQQSGPELVKP |
| | GASLKLSCTASGFNIKDTYIHWVKQRPEQCLEWIGRIYPTNGYTRYDP |
| | KFQDKATITADTSSNTAYLQVSRLTSEDTAVYYCSRWGGDGFYAMD |
| | YWGQGASVTVSSA |
| | (SEQ ID NO: 171) |

Alternatively, novel antigen-binding sites that can bind to HER2 can be identified by screening for binding to the amino acid sequence defined by SEQ ID NO:138 or a mature extracellular fragment thereof.

(SEQ ID NO: 138)
MELAALCRWGLLLALLPPGAASTQVCTGTDMKLRLPASPETHLDMLRHL
YQGCQVVQGNLELTYLPTNASLSFLQDIQEVQGYVLIAHNQVRQVPLQR
LRIVRGTQLFEDNYALAVLDNGDPLNNTTPVTGASPGGLRELQLRSLTE
ILKGGVLIQRNPQLCYQDTILWKDIFHKNNQLALTLIDTNRSRACHPCS
PMCKGSRCWGESSEDCQSLTRTVCAGGCARCKGPLPTDCCHEQCAAGCT
GPKHSDCLACLHFNHSGICELHCPALVTYNTDTFESMPNPEGRYTFGAS
CVTACPYNYLSTDVGSCTLVCPLHNQEVTAEDGTQRCEKCSKPCARVCY
GLGMEHLREVRAVTSANIQEFAGCKKIFGSLAFLPESFDGDPASNTAPL
QPEQLQVFETLEEITGYLYISAWPDSLPDLSVFQNLQVIRGRILHNGAY
SLTLQGLGISWLGLRSLRELGSGLALIHHNTHLCFVHTVPWDQLFRNPH
QALLHTANRPEDECVGEGLACHQLCARGHCWGPGPTQCVNCSQFLRGQE
CVEECRVLQGLPREYVNARHCLPCHPECQPQNGSVTCFGPEADQCVACA
HYKDPPFCVARCPSGVKPDLSYMPIWKFPDEEGACQPCPINCTHSCVDL
DDKGCPAEQRASPLTSIISAVVGILLVVVLGVVFGILIKRRQQKIRKYT
MRRLLQETELVEPLTPSGAMPNQAQMRILKETELRKVKVLGSGAFGTVY
KGIWIPDGENVKIPVAIKVLRENTSPKANKEILDEAYVMAGVGSPYVSR
LLGICLTSTVQLVTQLMPYGCLLDHVRENRGRLGSQDLLNWCMQIAKGM
SYLEDVRLVHRDLAARNVLVKSPNHVKITDFGLARLLDIDETEYHADGG
KVPIKWMALESILRRRFTHQSDVWSYGVTVWELMTFGAKPYDGIPAREI
PDLLEKGERLPQPPICTIDVYMIMVKCWMIDSECRPRFRELVSEFSRMA
RDPQRFVVIQNEDLGPASPLDSTFYRSLLEDDDMGDLVDAEEYLVPQQG
FFCPDPAPGAGGMVHHRHRSSSTRSGGGDLTLGLEPSEEEAPRSPLAPS
EGAGSDVFDGDLGMGAAKGLQSLPTHDPSPLQRYSEDPTVPLPSETDGY
VAPLTCSPQPEYVNQPDVRPQPPSPREGPLPAARPAGATLERPKTLSPG
KNGVVKDVFAFGGAVENPEYLTPQGGAAPQPHPPPAFSPAFDNLYYWDQ
DPPERGAPPSTFKGTPTAENPEYLGLDVPV.

The VH and VL of the scFv can be positioned in various orientations. In certain embodiments, the VL is positioned N-terminal to the VH. In certain embodiments, the VL is positioned C-terminal to the VH.

The VH and VL of the scFv can be connected via a linker, e.g., a peptide linker. In certain embodiments, the peptide linker is a flexible linker. Regarding the amino acid composition of the linker, peptides are selected with properties that confer flexibility, do not interfere with the structure and function of the other domains of the proteins of the present invention, and resist cleavage from proteases. For example, glycine and serine residues generally provide protease resistance. In certain embodiments, the VL is positioned N-terminal to the VH and is connected to the VH via a linker.

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-25, 15-20, 20-50, 20-40, 20-30, or 20-25 amino acid residues in length.

In certain embodiments, the linker comprises or consists of a (GS)ₙ (SEQ ID NO: 204), (GGS)ₙ (SEQ ID NO:205), (GGGS)ₙ (SEQ ID NO:206), (GGSG)ₙ (SEQ ID NO: 207), (GGSGG)ₙ (SEQ ID NO:208), and (GGGGS)ₙ (SEQ ID NO:209) sequence, wherein n is 1, 2, 3, 4, 5, 6, 7, 8, 9, 10, 11, 12, 13, 14, 15, 16, 17, 18, 19, or 20. In certain embodiments, the linker comprises or consists of an amino acid sequence selected from SEQ ID NO: 143, SEQ ID NO:201, SEQ ID NO:202, SEQ ID NO: 103, SEQ ID NO: 104, SEQ ID NO: 83, SEQ ID NO:84, SEQ ID NO: 150, SEQ ID NO:152, and SEQ ID NO: 154, as listed in Table 3. In certain embodiments, the linker is a (G₄S)₄ (SEQ ID NO:143) linker consisting of the sequence of SEQ ID NO:143.

TABLE 3

| SEQ ID | Amino Acid Sequence |
|---|---|
| SEQ ID | GSGSGSGSGSGSGSGSGSGS |
| NO: 201 | |
| SEQ ID | GGSGGSGGSGGSGGSGGSGGSGGSGGSGGS |
| NO: 202 | |
| SEQ ID | GGGSGGGSGGGSGGGSGGGSGGGSGGGSG |
| NO: 103 | GGSGGGSGGGS |
| SEQ ID | GGSGGGSGGGSGGGSGGGSGGGSGGGSGG |
| NO: 104 | GSGGGSGGGSG |
| SEQ ID | GGSGGGGSGGGGSGGGGSGGGGSGGGGSG |
| NO: 83 | GGGSGGGGSGGGGSGGGGSGG |
| SEQ ID | GGGGSGGGGSGGGGSGGGGSGGGGSGGGG |
| NO: 84 | SGGGGSGGGGSGGGGSGGGGS |
| SEQ ID | GGGGSGGGGSGGGGSGGGGS |
| NO: 143 | |
| SEQ ID | GGGGSGGGGSGGGGS |
| NO: 150 | |
| SEQ ID | GGGGSGGGGSGGGGSGGGGSGGGGSGGGG |
| NO: 152 | SGGGGSGGGGSGGGGSGGGGSGGGGSGGG |
| | GSGGGGSGGGGSGGGGSGGGGSGGGGSGG |
| | GGSGGGGSGGGGS |
| SEQ ID | GGSGGGGSGGGGSGGGGSGGGGSGGGGSG |
| NO: 154 | GGGSGGGGSGGGGSGGGGSGGGGSGGGGS |
| | GGGGSGGGGSGGGGSGGGGSGGGGSGGGG |
| | SGGGGSGGGGSGG |

### Fc Domain

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al, Nature, 406 (6793): 267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine(S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as a human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the C_{κ} of a human IgG1 constant region may be at amino acid E123, F116, S176, V163, S174, and/or T164.

Amino acid substitutions could be selected from the following sets of substitutions shown in Table 4.

TABLE 4

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 5.

TABLE 5

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 6.

TABLE 6

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 7.

TABLE 7

| First Polypeptide | Second Polypeptide |
|---|---|
| L351Y, D399R, D399K, | T366V, T366I, T366L, T366M, N390D, |
| S400K, S400R, | N390E, K392L, K392M, K392V, K392F |
| Y407A, Y407I, Y407V | K392D, K392E, K409F, K409W, T411D |
| | and T411E |

Alternatively, at least one amino acid substitutions could be selected from the following set of substitutions in Table 8, where the position(s) indicated in the First Polypeptide column is replaced by any known negatively-charged amino acid, and the position(s) indicated in the Second Polypeptide Column is replaced by any known positively-charged amino acid.

TABLE 8

| First Polypeptide | Second Polypeptide |
|---|---|
| K392, K370, K409, or K439 | D399, E356, or E357 |

Alternatively, at least one amino acid substitutions could be selected from the following set of in Table 9, where the position(s) indicated in the First Polypeptide column is replaced by any known positively-charged amino acid, and the position(s) indicated in the Second Polypeptide Column is replaced by any known negatively-charged amino acid.

TABLE 9

| First Polypeptide | Second Polypeptide |
|---|---|
| D399, E356, or E357 | K409, K439, K370, or K392 |

Alternatively, amino acid substitutions could be selected from the following set in Table 10.

TABLE 10

| First Polypeptide | Second Polypeptide |
|---|---|
| T350V, L351Y, F405A, and Y407V | T350V, T366L, K392L, and T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, S400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, S400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

Certain proteins described in the present disclosure have an Fc domain, which comprises one or more mutations that reduce the ability of the Fc sequence to induce antibody-dependent cellular cytotoxicity (ADCC) and/or antibody-dependent cellular phagocytosis (ADCP). At least one mutation is located in the regions including amino acid positions 232-239, 265-270, 296-299, and 325-332 (see Want et al., Protein Cell (2018) 9(1): 63-73). The mutations may include an amino acid substitution (relative to wild-type human IgG1) at one or more positions 233, 234, 235, 297, and 329. The one or more mutations may include E233P; L234A; L235A; N297A, N297Q, N297G, or N297D; and/or P329A, P329G, or P329R relative to wild-type human IgG1. The one or more mutations may include L234A and L235A relative to wild-type human IgG1. Alternatively, the one or more mutations may include L234A, L235A, and P329A relative to wild-type human IgG1. The mutation may be present on each of the two polypeptide chains of the Fc domain.

### Exemplary Multi-Specific Binding Proteins

Listed below are examples of TriNKETs comprising a HER2-binding scFv and an NKG2D-binding Fab each linked to an antibody constant region, wherein the antibody constant regions include mutations that enable heterodimerization of two Fc chains. The scFv comprises a heavy chain variable domain (VH) and a light chain variable domain (VL) derived from an anti-HER2 antibody (e.g., trastuzumab), and further comprises substitution of Cys for the amino acid residues at position 100 of VL and position 44 of VH, thereby facilitating formation of a disulfide bridge between the VH and VL of the scFv. The VL is linked N-terminal to the VH via a (G₄S)₄ linker (SEQ ID NO:143), and the VH is linked N-terminal to an Fc via an Ala-Ser linker. The Ala-Ser linker is included at the elbow hinge region sequence to balance between flexibility and optimal geometry. In certain embodiments, an additional sequence Thr-Lys-Gly can be added N-terminal or C-terminal to the Ala-Ser sequence at the hinge. As used herein to describe these exemplary TriNKETs, Fc includes an antibody hinge, CH2, and CH3.

Accordingly, each of the TriNKETs described below comprises the following three polypeptide chains:
Chain A, comprising from N-terminus to C-terminus: VH of an NKG2D-binding Fab, CH1, and Fc;
Chain B, comprising from N-terminus to C-terminus: VL of a HER2-binding scFv, (G₄S)₄ linker (SEQ ID NO:143), VH of the HER2-binding scFv, Ala-Ser linker, and Fc; and
Chain C, comprising from N-terminus to C-terminus: VL of the NKG2D-binding Fab, and CL.

The amino acid sequences of the exemplary TriNKETs are summarized in Table

TABLE 11

| | NKG2D | HER2 | Human | | | |
|---|---|---|---|---|---|---|
| TriNKET | Binding | Binding | IgG1 | Chain | Chain | Chain |
| Construct | Fab | scFv | Fc | A | B | C |
| A49-F3'- | A49 | Trastuzumab | EW- | SEQ | SEQ | SEQ |
| TriNKET- | | | RVT | ID | ID | ID |
| Trastuzumab | | | | NO: | NO: | NO: |
| | | | | 141 | 140 | 142 |
| A49-F3'- | A49 | Trastuzumab | KIH | SEQ | SEQ | SEQ |
| KiH- | | | | ID | ID | ID |
| TriNKET- | | | | NO: | NO: | NO: |
| Trastuzumab | | | | 147 | 146 | 142 |
| A49-F3'- | A49 | Pertuzumab | EW- | SEQ | SEQ | SEQ |
| TriNKET- | | | RVT | ID | ID | ID |
| Pertuzumab | | | | NO: | NO: | NO: |
| | | | | 141 | 190 | 142 |
| A49-F3'- | A49 | Pertuzumab | KIH | SEQ | SEQ | SEQ |
| KiH- | | | | ID | ID | ID |
| TriNKET- | | | | NO: | NO: | NO: |
| Pertuzumab | | | | 147 | 191 | 142 |
| A49-F3'- | A49 | MGAH22 | EW- | SEQ | SEQ | SEQ |
| TriNKET- | | | RVT | ID | ID | ID |
| MGAH22 | | | | NO: | NO: | NO: |
| | | | | 141 | 192 | 142 |
| A49-F3'- | A49 | MGAH22 | KIH | SEQ | SEQ | SEQ |
| KiH- | | | | ID | ID | ID |
| TriNKET- | | | | NO: | NO: | NO: |
| MGAH22 | | | | 147 | 193 | 142 |
| A49MI-F3'- | A49MI | Trastuzumab | EW- | SEQ | SEQ | SEQ |
| TriNKET- | | | RVT | ID | ID | ID |
| Trastuzumab | | | | NO: | NO: | NO: |
| | | | | 145 | 140 | 142 |
| A49MI-F3'- | A49MI | Trastuzumab | KIH | SEQ | SEQ | SEQ |
| KiH- | | | | ID | ID | ID |
| TriNKET- | | | | NO: | NO: | NO: |
| Trastuzumab | | | | 194 | 146 | 142 |
| A49MI-F3'- | A49MI | Pertuzumab | EW- | SEQ | SEQ | SEQ |
| TriNKET- | | | RVT | ID | ID | ID |
| Pertuzumab | | | | NO: | NO: | NO: |
| | | | | 145 | 190 | 142 |
| A49MI-F3'- | A49MI | Pertuzumab | KIH | SEQ | SEQ | SEQ |
| KiH- | | | | ID | ID | ID |
| TriNKET- | | | | NO: | NO: | NO: |
| Pertuzumab | | | | 194 | 191 | 142 |
| A49MI-F3'- | A49MI | MGAH22 | EW- | SEQ | SEQ | SEQ |
| TriNKET- | | | RVT | ID | ID | ID |
| MGAH22 | | | | NO: | NO: | NO: |
| | | | | 145 | 192 | 142 |
| A49MI-F3'- | A49MI | MGAH22 | KIH | SEQ | SEQ | SEQ |
| KiH- | | | | ID | ID | ID |
| TriNKET- | | | | NO: | NO: | NO: |
| MGAH22 | | | | 194 | 193 | 142 |
| A44-F3'- | A44 | Trastuzumab | EW- | SEQ | SEQ | SEQ |
| TriNKET- | | | RVT | ID | ID | ID |
| Trastuzumab | | | | NO: | NO: | NO: |
| | | | | 155 | 140 | 149 |
| A44-F3'- | A44 | Trastuzumab | KIH | SEQ | SEQ | SEQ |
| KiH- | | | | ID | ID | ID |
| TriNKET- | | | | NO: | NO: | NO: |
| Trastuzumab | | | | 148 | 146 | 149 |
| A44-F3'- | A44 | Pertuzumab | EW- | SEQ | SEQ | SEQ |
| TriNKET- | | | RVT | ID | ID | ID |
| Pertuzumab | | | | NO: | NO: | NO: |
| | | | | 155 | 190 | 149 |
| A44-F3'- | A44 | Pertuzumab | KIH | SEQ | SEQ | SEQ |
| KiH- | | | | ID | ID | ID |
| TriNKET- | | | | NO: | NO: | NO: |
| Pertuzumab | | | | 148 | 191 | 149 |
| A44-F3'- | A44 | MGAH22 | EW- | SEQ | SEQ | SEQ |
| TriNKET- | | | RVT | ID | ID | ID |
| MGAH22 | | | | NO: | NO: | NO: |
| | | | | 155 | 192 | 149 |
| A44-F3'- | A44 | MGAH22 | KIH | SEQ | SEQ | SEQ |
| KiH- | | | | ID | ID | ID |
| TriNKET- | | | | NO: | NO: | NO: |
| MGAH22 | | | | 148 | 193 | 149 |

In certain embodiments, the multi-specific binding protein of the present disclosure comprises a first polypeptide chain, a second polypeptide chain, and a third polypeptide chain, wherein the first, second, and third polypeptide chains comprise the amino acid sequences of Chain A, Chain B, and Chain C, respectively, of a TriNKET disclosed in Table 11. In certain embodiments, the first, second, and third polypeptide chains consist of the amino acid sequences of Chain A, Chain B, and Chain C, respectively, of a TriNKET disclosed in Table 11.

In an exemplary embodiment, the Fc domain linked to the NKG2D-binding Fab fragment comprises the mutations of Q347R, D399V, and F405T, and the Fc domain linked to the HER2 scFv comprises matching mutations K360E and K409W for forming a heterodimer. In another exemplary embodiment, the Fc domain linked to the NKG2D-binding Fab fragment comprises knob mutations T366S, L368A, and Y407V, and the Fc domain linked to the HER2-binding scFv comprises a “hole” mutation T366W. In an exemplary embodiment, the Fc domain linked to the NKG2D-binding Fab fragment includes an S354C substitution in the CH3 domain, which forms a disulfide bond with a Y349C substitution on the Fc linked to the HER2-binding scFv.

Specific TriNKETs and their polypeptide chains are described in more detail below. In the amino acid sequences, (G₄S)₄ (SEQ ID NO:143) and Ala-Ser linkers are bold-underlined; Cys residues in scFv that form disulfide bridges are bold-italic-underlined; Fc heterodimerization mutations are bold-underlined; and CDR sequences under Kabat are underlined.

For example, a TriNKET of the present disclosure is A49-F3′-TriNKET-Trastuzumab. A49-F3′-TriNKET-Trastuzumab includes a single-chain variable fragment (scFv) (SEQ ID NO:139) derived from trastuzumab that binds HER2, linked via a hinge comprising Ala-Ser to an Fc domain; and an NKG2D-binding Fab fragment derived from A49 including a heavy chain portion comprising a heavy chain variable domain (SEQ ID NO: 94) and a CH1 domain, and a light chain portion comprising a light chain variable domain (SEQ ID NO:98) and a light chain constant domain, wherein the heavy chain variable domain is connected to the CH1 domain, and the CH1 domain is connected to the Fc domain. A49-F3′-TriNKET-Trastuzumab includes three polypeptides, having the sequences of SEQ ID NO: 140, SEQ ID NO: 141, and SEQ ID NO:142.

SEQ ID NO:140 represents the full sequence of the HER2-binding scFv linked to an Fc domain via a hinge comprising Ala-Ser (scFv-Fc). The Fc domain linked to the scFv includes Q347R, D399V, and F405T substitutions for heterodimerization and an S354C substitution for forming a disulfide bond with a Y349C substitution in SEQ ID NO:141 as described below. The scFv (SEQ ID NO:139) includes a heavy chain variable domain of trastuzumab connected to the N-terminus of a light chain variable domain of trastuzumab via a (G₄S)₄ linker (SEQ ID NO:143), the scFv represented as VL-(G₄S)₄-VH (“(G₄S)₄” is represented by SEQ ID NO: 143). The heavy and the light variable domains of the scFv are also connected through a disulfide bridge between C100 of VL and C44 of VH, as a result of Q100C and G44C substitutions in the VL and VH, respectively.

Trastuzumab scFv
(SEQ ID NO: 139)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GCGTKVEIKGGGGSGGGGSGGGGSGGGGSEVQLVESGGGLVQPGGSLRL
SCAASGFNIKDTYIHWVRQAPGKCLEWVARIYPTNGYTRYADSVKGRFT
ISADTSKNTAYLQMNSLRAEDTAVYYCSRWGGDGFYAMDYWGQGTLVTV
SS
Trastuzumab scFv-Fc (RVT)
(SEQ ID NO: 140)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GCGTKVEIKGGGGSGGGGSGGGGSGGGGSEVQLVESGGGLVQPGGSLRL
SCAASGFNIKDTYIHWVRQAPGKCLEWVARIYPTNGYTRYADSVKGRFT
ISADTSKNTAYLQMNSLRAEDTAVYYCSRWGGDGFYAMDYWGQGTLVTV
SSASDKTHTCPPCPAPELLGGPSVFLFPPKPKDTLMISRTPEVTCVVVD
VSHEDPEVKENWYVDGVEVHNAKTKPREEQYNSTYRVVSVLTVLHQDWL
NGKEYKCKVSNKALPAPIEKTISKAKGQPREPRVYTLPPCRDELTKNQV
SLTCLVKGFYPSDIAVEWESNGQPENNYKTTPPVLVSDGSFTLYSKLTV
DKSRWQQGNVFSCSVMHEALHNHYTQKSLSLSPG

SEQ ID NO:141 represents the heavy chain portion of the Fab fragment, which comprises a heavy chain variable domain (SEQ ID NO:94) of an NKG2D-binding site and a CH1 domain, connected to an Fc domain. The Fc domain in SEQ ID NO:141 includes a Y349C substitution in the CH3 domain, which forms a disulfide bond with an S354C substitution on the Fc linked to the HER2-binding scFv (SEQ ID NO:140). In SEQ ID NO: 141, the Fc domain also includes K360E and K409W substitutions for heterodimerization with the Fc in SEQ ID NO:140.

A49 VH
(SEQ ID NO: 94)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAPMGAAAGWFDPWGQGTLVTVSS
A49 VH-CH1-Fc (EW)
(SEQ ID NO: 141)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAPMGAAAGWFDPWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVCTLPPSRDELTENQVSLTCLVKGFYPSDIAVEWESNGQP
ENNYKTTPPVLDSDGSFFLYSWLTVDKSRWQQGNVFSCSVMHEALHNHY
TQKSLSLSPG

SEQ ID NO:142 represents the light chain portion of the Fab fragment comprising a light chain variable domain (SEQ ID NO:98) of an NKG2D-binding site and a light chain constant domain.

A49 VL
(SEQ ID NO: 98)
DIQMTQSPSSVSASVGDRVTITCRASQGISSWLAWYQQKPGKAPKLLIY
AASSLQSGVPSRFSGSGSGTDFTLTISSLQPEDFATYYCQQGVSFPRTF
GGGTKVEIK
A49 VL-LC
(SEQ ID NO: 142)
DIQMTQSPSSVSASVGDRVTITCRASQGISSWLAWYQQKPGKAPKLLIY
AASSLQSGVPSRFSGSGSGTDFTLTISSLQPEDFATYYCQQGVSFPRTF
GGGTKVEIKRTVAAPSPPSDEQLKSGTASVVCLLNNFYPREAKVQWKVD
NALQSGNSQESVTEQDSKDSTYSLSSTLTLSKADYEKHKVYACEVTHQG
LSSPVTKSFNRGEC

Another TriNKET of the present disclosure is A49MI-F3′-TriNKET-Trastuzumab. A49MI-F3′-TriNKET-Trastuzumab includes the same Her2-binding scFv (SEQ ID NO:139) as in A49-F3′-TriNKET-Trastuzumab linked via a hinge comprising Ala-Ser to an Fc domain; and an NKG2D-binding Fab fragment derived from A49MI including a heavy chain portion comprising a heavy chain variable domain (SEQ ID NO:144) and a CH1 domain, and a light chain portion comprising a light chain variable domain (SEQ ID NO:98) and a light chain constant domain, wherein the heavy chain variable domain is connected to the CH1 domain, and the CH1 domain is connected to the Fc domain. A49MI-F3′-TriNKET-Trastuzumab includes three polypeptides, having the sequences of SEQ ID NO:140 (as in A49-F3′-TriNKET-Trastuzumab), SEQ ID NO:145, and SEQ ID NO: 142 (as in A49-F3′-TriNKET-Trastuzumab).

SEQ ID NO:145 represents a heavy chain portion of the Fab fragment, which comprises a heavy chain variable domain (SEQ ID NO:144) of an NKG2D-binding site and a CH1 domain, connected to an Fc domain. In SEQ ID NO:144, wherein a methionine in the CDR3 of SEQ ID NO:94 has been substituted by isoleucine (M→I substitution; shown within a third bracket [ ] in SEQ ID NO:144 and SEQ ID NO:145). The Fc domain in SEQ ID NO: 145 includes a Y349C substitution in the CH3 domain, which forms a disulfide bond with an S354C substitution in the Fc linked to the HER2-binding scFv (SEQ ID NO:140). In SEQ ID NO:145, the Fc domain also includes K360E and K409W substitutions.

A49MI VH
(SEQ ID NO: 144)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAP[I]GAAAGWEDPWGQGTLVTVSS
A49MI VH-CH1-Fc (EW)
(SEQ ID NO: 145)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAP[I]GAAAGWFDPWGQGTLVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTI
SKAKGQPREPQVCTLPPSRDELTENQVSLTCLVKGFYPSDIAVEWESNG
QPENNYKTTPPVLDSDGSFFLYSWLTVDKSRWQQGNVFSCSVMHEALHN
HYTQKSLSLSPG

Another TriNKET of the present disclosure is A49-F3′-KiH-TriNKET-Trastuzumab. KiH refers to the knobs-into-holes (KiH) Fc technology, which involves engineering of the CH3 domains to create either a “knob” or a “hole” in each heavy chain to promote heterodimerization. The concept behind the KiH Fc technology was to introduce a “knob” in one CH3 domain (CH3A) by substitution of a small residue with a bulky one (e.g., T366WCH3A in EU numbering). To accommodate the “knob,” a complementary “hole” surface was created on the other CH3 domain (CH3B) by replacing the closest neighboring residues to the knob with smaller ones (e.g., T366S/L368A/Y407VCH3B). The “hole” mutation was optimized by structured-guided phage library screening (Atwell S, Ridgway J B, Wells J A, Carter P., Stable heterodimers from remodeling the domain interface of a homodimer using a phage display library, J. Mol. Biol. (1997) 270 (l): 26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426 (9): 1947-57; Mimoto F, Kadono S, Katada H, Igawa T, Kamikawa T, Hattori K. Crystal structure of a novel asymmetrically engineered Fc variant with improved affinity for FcγRs. Mol. Immunol. (2014) 58 (l): 132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

A49-F3′-KiH-TriNKET-Trastuzumab includes the same Her2-binding scFv (SEQ ID NO: 139) as in A49-F3′-TriNKET-Trastuzumab linked via a hinge comprising Ala-Ser to an Fc domain comprising the “hole” substitutions of T366S, L368A, and Y407V; and the same NKG2D-binding Fab fragment as in A49-F3′-TriNKET-Trastuzumab, the CH1 domain of which is connected to an Fc domain comprising the “knob” substitution of T366W. A49-F3′-KiH-TriNKET-Trastuzumab includes three polypeptides, having the sequences of SEQ ID NO: 146, SEQ ID NO: 147, and SEQ ID NO: 142 (as in A49-F3′-TriNKET-Trastuzumab).

SEQ ID NO: 146 represents the full sequence of the HER2-binding scFv (SEQ ID NO: 139) linked to an Fc domain via a hinge comprising Ala-Ser (scFv-Fc). The Fc domain linked to the scFv includes T366S, L368A, and Y407V substitutions for heterodimerization and an S354C substitution for forming a disulfide bond with a Y349C substitution in SEQ ID NO: 147 as described below.

Trastuzumab scFv-Fc (KiH)
(SEQ ID NO: 146)
DIQMTQSPSSLSASVGDRVTITCRASQDVNTAVAWYQQKPGKAPKLLIY
SASFLYSGVPSRFSGSRSGTDFTLTISSLOPEDFATYYCQQHYTTPPTF
GCGTKVEIKGGGGSGGGGSGGGGSGGGGSEVQLVESGGGLVQPGGSLRL
SCAASGFNIKDTYIHWVRQAPGKCLEWVARIYPTNGYTRYADSVKGRFT
ISADTSKNTAYLQMNSLRAEDTAVYYCSRWGGDGFYAMDYWGQGTLVTV
SSASDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVCTLPPSRDELTKNQV
SLSCAVKGFYPSDIAVEWESNGQPENNYKTTPPVLDSDGSFFLVSKLTV
DKSRWQQGNVFSCSVMHEALHNHYTQKSLSLSPG

SEQ ID NO:147 represents the heavy chain portion of a Fab fragment, which comprises a heavy chain variable domain (SEQ ID NO:94) of an NKG2D-binding site derived from A49 and a CH1 domain, connected to an Fc domain. The Fc domain in SEQ ID NO: 147 includes an S354C substitution, which forms a disulfide bond with a Y349C substitution in the CH3 domain of the Fc linked to the HER2-binding scFv (SEQ ID NO: 146). In SEQ ID NO:147, the Fc domain also includes a T366W substitution.

A49 VH-CH1-Fc (KiH)
(SEQ ID NO: 147)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAPMGAAAGWFDPWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPCRDELTKNQVSLWCLVKGFYPSDIAVEWESNGQP
ENNYKTTPPVLDSDGSFFLYSKLTVDKSRWQQGNVFSCSVMHEALHNHY
TQKSLSLSPG

Another TriNKET of the present disclosure is A49MI-F3′-KiH-TriNKET-Trastuzumab. A49MI-F3′-KiH-TriNKET-Trastuzumab includes the same Her2-binding scFv (SEQ ID NO:139) as in A49-F3′-TriNKET-Trastuzumab linked via a hinge comprising Ala-Ser to an Fc domain comprising the “hole” substitutions of T366S, L368A, and Y407V; and the same NKG2D-binding Fab fragment as in A49MI-F3′-TriNKET-Trastuzumab, the CH1 domain of which is connected to an Fc domain comprising the “knob” substitution of T366W. A49MI-F3′-KiH-TriNKET-Trastuzumab includes three polypeptides, having the sequences of SEQ ID NO:146 (as in A49-F3′-KiH-TriNKET-Trastuzumab), SEQ ID NO: 194, and SEQ ID NO:142 (as in A49-F3′-TriNKET-Trastuzumab).

SEQ ID NO:194 represents the heavy chain portion of a Fab fragment, which comprises a heavy chain variable domain (SEQ ID NO:144) of an NKG2D-binding site derived from A49MI and a CH1 domain, connected to an Fc domain. The Fc domain in SEQ ID NO: 194 includes an S354C substitution, which forms a disulfide bond with a Y349C substitution in the CH3 domain of the Fc linked to the HER2-binding scFv (SEQ ID NO: 146). In SEQ ID NO:194, the Fc domain also includes a T366W substitution.

A49MI VH-CH1-Fc (KiH)
(SEQ ID NO: 194)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAPIGAAAGWFDPWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPCRDELTKNQVSLWCLVKGFYPSDIAVEWESNGQP
ENNYKTTPPVLDSDGSFFLYSKLTVDKSRWQQGNVFSCSVMHEALHNHY
TQKSLSLSPG

Another exemplary TriNKET of the present disclosure is A44-F3′-TriNKET-Trastuzumab. A44-F3′-TriNKET-Trastuzumab includes the same Her2-binding scFv (SEQ ID NO: 139) as in A49-F3′-TriNKET-Trastuzumab linked via a hinge comprising Ala-Ser to an Fc domain; and an NKG2D-binding Fab fragment derived from A44 including a heavy chain portion comprising a heavy chain variable domain (SEQ ID NO:86) and a CH1 domain, and a light chain portion comprising a light chain variable domain (SEQ ID NO:90) and a light chain constant domain, wherein the heavy chain variable domain is connected to the CH1 domain, and the CH1 domain is connected to the Fc domain. A44-F3′-TriNKET-Trastuzumab includes three polypeptides, having the sequences of SEQ ID NO:140 (as in A49-F3′-TriNKET-Trastuzumab), SEQ ID NO: 155, and SEQ ID NO:149.

SEQ ID NO:155 represents a heavy chain variable domain (SEQ ID NO:86) of an NKG2D-binding site derived from A44, connected to an Fc domain. The Fc domain in SEQ ID NO: 155 includes a Y349C substitution in the CH3 domain, which forms a disulfide bond with an S354C substitution on the Fc linked to the HER2-binding scFv (SEQ ID NO:140). In SEQ ID NO:155, the Fc domain also includes K360E and K409W substitutions.

A44 VH
(SEQ ID NO: 86)
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DGGYYDSGAGDYWGQGTLVTVSS
A44 VH-CH1-Fc (EW)
(SEQ ID NO: 155)
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DGGYYDSGAGDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVCTLPPSRDELTENQVSLTCLVKGFYPSDIAVEWESNGQPE
NNYKTTPPVLDSDGSFFLYSWLTVDKSRWQQGNVFSCSVMHEALHNHYT
QKSLSLSPG

SEQ ID NO:149 represents the light chain portion of the Fab fragment comprising a light chain variable domain (SEQ ID NO:90) of an NKG2D-binding site and a light chain constant domain.

A44 VL
(SEQ ID NO: 90)
DIQMTQSPSSVSASVGDRVTITCRASQGIDSWLAWYQQKPGKAPKLLIY
AASSLQSGVPSRFSGSGSGTDFTLTISSLQPEDFATYYCQQGVSYPRTF
GGGTKVEIK
A44 VL-CL
(SEQ ID NO: 149)
DIQMTQSPSSVSASVGDRVTITCRASQGIDSWLAWYQQKPGKAPKLLIY
AASSLQSGVPSRFSGSGSGTDFTLTISSLQPEDFATYYCQQGVSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Another exemplary TriNKET of the present disclosure is A44-F3′-KiH-TriNKET-Trastuzumab. A44-F3′-KiH-TriNKET-Trastuzumab includes the same Her2-binding scFv (SEQ ID NO: 139) as in A49-F3′-TriNKET-Trastuzumab linked via a hinge comprising Ala-Ser to an Fc domain comprising the “hole” substitutions of T366S, L368A, and Y407V; and the same NKG2D-binding Fab fragment as in A44-F3′-TriNKET-Trastuzumab, the CH1 domain of which is connected to an Fc domain comprising the “knob” substitution of T366W. A44-F3′-KiH-TriNKET-Trastuzumab includes three polypeptides, having the sequences of SEQ ID NO:146 (as in A49-F3′-KiH-TriNKET-Trastuzumab), SEQ ID NO: 148, and SEQ ID NO: 149 (as in A44-F3′-TriNKET-Trastuzumab).

SEQ ID NO:148 represents a heavy chain variable domain (SEQ ID NO:86) of an NKG2D-binding site derived from A44, connected to an Fc domain. The Fc domain in SEQ ID NO:148 includes a Y349C substitution in the CH3 domain, which forms a disulfide bond with an S354C substitution on the Fc linked to the HER2-binding scFv (SEQ ID NO: 146). In SEQ ID NO:148, the Fc domain also includes a T366W substitution.

A44 VH-CHI-Fc (KiH)
(SEQ ID NO: 148)
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DGGYYDSGAGDYWGQGTLVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPCRDELTKNQVSLWCLVKGFYPSDIAVEWESNGQPE
NNYKTTPPVLDSDGSFFLYSKLTVDKSRWQQGNVFSCSVMHEALHNHYT
QKSLSLSPG

Another TriNKET of the present disclosure is A49-F3′-TriNKET-Pertuzumab. A49-F3′-TriNKET-Pertuzumab includes an scFv (SEQ ID NO:189) derived from pertuzumab that binds HER2, linked via a hinge comprising Ala-Ser to an Fc domain; and and the same NKG2D-binding Fab fragment as in A49-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A49-F3′-TriNKET-Pertuzumab includes three polypeptides, having the sequences of SEQ ID NO: 190, SEQ ID NO:141 (as in A49-F3′-TriNKET-Trastuzumab), and SEQ ID NO:142 (as in A49-F3′-TriNKET-Trastuzumab).

SEQ ID NO: 190 represents the full sequence of the HER2-binding scFv linked to an Fc domain via a hinge comprising Ala-Ser (scFv-Fc). The Fc domain linked to the scFv includes Q347R, D399V, and F405T substitutions for heterodimerization and an S354C substitution for forming a disulfide bond with a Y349C substitution in SEQ ID NO:141 as described above. The scFv (SEQ ID NO:189) includes a heavy chain variable domain of pertuzumab connected to the N-terminus of a light chain variable domain of pertuzumab via a (G₄S)₄ linker (SEQ ID NO:143), the scFv represented as VL-(G₄S)₄-VH (“(G₄S)₄” is represented by SEQ ID NO: 143). The heavy and the light variable domains of the scFv are also connected through a disulfide bridge between C100 of VL and C44 of VH, as a result of Q100C and G44C substitutions in the VL and VH, respectively.

Pertuzumab scFv
(SEQ ID NO: 189)
DIQMTQSPSSLSASVGDRVTITCKASQDVSIGVAWYQQKPGKAPKLLIY
SASYRYTGVPSRFSGSGSGTDFTLTISSLQPEDFATYYCQQYYIYPYTF
GCGTKVEIKRGGGGSGGGGSGGGGSGGGGSEVQLVESGGGLVQPGGSLR
LSCAASGFTFTDYTMDWVRQAPGKCLEWVADVNPNSGGSIYNQRFKGRF
TLSVDRSKNTLYLQMNSLRAEDTAVYYCARNLGPSFYFDYWGQGTLVTV
SSA
Pertuzumab scFv-Fc
(SEQ ID NO: 190)
DIQMTQSPSSLSASVGDRVTITCKASQDVSIGVAWYQQKPGKAPKLLIY
SASYRYTGVPSRFSGSGSGTDFTLTISSLQPEDFATYYCQQYYIYPYTF
GCGTKVEIKRGGGGSGGGGSGGGGSGGGGSEVQLVESGGGLVQPGGSLR
LSCAASGFTFTDYTMDWVRQAPGKCLEWVADVNPNSGGSIYNQRFKGRF
TLSVDRSKNTLYLQMNSLRAEDTAVYYCARNLGPSFYFDYWGQGTLVTV
SSAASDKTHTCPPCPAPELLGGPSVFLFPPKPKDTLMISRTPEVTCVVV
DVSHEDPEVKENWYVDGVEVHNAKTKPREEQYNSTYRVVSVLTVLHQDW
LNGKEYKCKVSNKALPAPIEKTISKAKGQPREPRVYTLPPCRDELTKNQ
VSLTCLVKGFYPSDIAVEWESNGQPENNYKTTPPVLVSDGSFTLYSKLT
VDKSRWQQGNVFSCSVMHEALHNHYTQKSLSLSPG

Another exemplary TriNKET of the present disclosure is A49MI-F3′-TriNKET-Pertuzumab. A49MI-F3′-TriNKET-Pertuzumab includes the same Her2-binding scFv (SEQ ID NO: 189) as in A49-F3′-TriNKET-Pertuzumab linked via a hinge comprising Ala-Ser to an Fc domain; and the same NKG2D-binding Fab fragment as in A49MI-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A49MI-F3′-TriNKET-Pertuzumab includes three polypeptides, having the sequences of SEQ ID NO: 190 (as in A49-F3′-KiH-TriNKET-Pertuzumab), SEQ ID NO: 145 (as in A49MI-F3′-TriNKET-Trastuzumab), and SEQ ID NO:142 (as in A49-F3′-TriNKET-Trastuzumab).

Another exemplary TriNKET of the present disclosure is A49-F3′-KiH-TriNKET-Pertuzumab. A49-F3′-KiH-TriNKET-Pertuzumab includes the same Her2-binding scFv (SEQ ID NO: 189) as in A49-F3′-TriNKET-Pertuzumab linked via a hinge comprising Ala-Ser to an Fc domain; and the same NKG2D-binding Fab fragment as in A49-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A49-F3′-KiH-TriNKET-Pertuzumab includes three polypeptides, having the sequences of SEQ ID NO:191, SEQ ID NO:147 (as in A49-F3′-KiH-TriNKET-Trastuzumab), and SEQ ID NO: 142 (as in A49-F3′-TriNKET-Trastuzumab).

SEQ ID NO: 191 represents the full sequence of the HER2-binding scFv (SEQ ID NO: 189) linked to an Fc domain via a hinge comprising Ala-Ser (scFv-Fc). The Fc domain linked to the scFv includes T366S, L368A, and Y407V substitutions for heterodimerization and an S354C substitution for forming a disulfide bond with a Y349C substitution in SEQ ID NO: 191 as described above.

Pertuzumab scFv-Fc (KiH)
(SEQ ID NO: 191)
DIQMTQSPSSLSASVGDRVTITCKASQDVSIGVAWYQQKPGKAPKLLIY
SASYRYTGVPSRFSGSGSGTDFTLTISSLOPEDFATYYCQQYYIYPYTF
GCGTKVEIKRGGGGSGGGGGGGGSGGGGSEVQLVESGGGLVQPGGSLRL
SCAASGFTFTDYTMDWVRQAPGKCLEWVADVNPNSGGSIYNQRFKGRFT
LSVDRSKNTLYLQMNSLRAEDTAVYYCARNLGPSFYFDYWGQGTLVTVS
SAASDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVCTLPPSRDELTKNQV
SLSCAVKGFYPSDIAVEWESNGQPENNYKTTPPVLDSDGSFFLVSKLTV
DKSRWQQGNVFSCSVMHEALHNHYTQKSLSLSPG

Another exemplary TriNKET of the present disclosure is A49MI-F3′-KiH-TriNKET-Pertuzumab. A49MI-F3′-KiH-TriNKET-Pertuzumab includes the same Her2-binding scFv (SEQ ID NO: 189) as in A49-F3′-TriNKET-Pertuzumab linked via a hinge comprising Ala-Ser to an Fc domain; and the same NKG2D-binding Fab fragment as in A49MI-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A49MI-F3′-KiH-TriNKET-Pertuzumab includes three polypeptides, having the sequences of SEQ ID NO:191 (as in A49-F3′-KiH-TriNKET-Pertuzumab), SEQ ID NO:194 (as in A49MI-F3′-KiH-TriNKET-Trastuzumab), and SEQ ID NO:142 (as in A49-F3′-TriNKET-Trastuzumab).

Another exemplary TriNKET of the present disclosure is A44-F3′-TriNKET-Pertuzumab. A44-F3′-TriNKET-Pertuzumab includes the same Her2-binding scFv (SEQ ID NO: 189) as in A49-F3′-TriNKET-Pertuzumab linked via a hinge comprising Ala-Ser to an Fc domain; and the same NKG2D-binding Fab fragment as in A44-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A44-F3′-TriNKET-Pertuzumab includes three polypeptides, having the sequences of SEQ ID NO: 190 (as in A49-F3′-KiH-TriNKET-Pertuzumab), SEQ ID NO:155 (as in A44-F3′-TriNKET-Trastuzumab), and SEQ ID NO: 149 (as in A44-F3′-TriNKET-Trastuzumab).

Another exemplary TriNKET of the present disclosure is A44-F3′-KiH-TriNKET-Pertuzumab. A44-F3′-KiH-TriNKET-Pertuzumab includes the same Her2-binding scFv (SEQ ID NO:189) as in A49-F3′-TriNKET-Pertuzumab linked via a hinge comprising Ala-Ser to an Fc domain; and the same NKG2D-binding Fab fragment as in A44-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A44-F3′-KiH-TriNKET-Pertuzumab includes three polypeptides, having the sequences of SEQ ID NO: 191 (as in A49-F3′-KiH-TriNKET-Pertuzumab), SEQ ID NO:148 (as in A44-F3′-KiH-TriNKET-Trastuzumab), and SEQ ID NO:149 (as in A44-F3′-TriNKET-Trastuzumab).

Another TriNKET of the present disclosure is A49-F3′-TriNKET-MGAH22.

A49-F3′-TriNKET-MGAH22 includes an scFv (SEQ ID NO:171) derived from MGAH22 that binds HER2, linked via a hinge comprising Ala-Ser to an Fc domain; and the same NKG2D-binding Fab fragment as in A49-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A49-F3′-TriNKET-MGAH22 includes three polypeptides, having the sequences of SEQ ID NO:192, SEQ ID NO: 141 (as in A49-F3′-TriNKET-Trastuzumab), and SEQ ID NO:142 (as in A49-F3′-TriNKET-Trastuzumab).

SEQ ID NO:192 represents the full sequence of the HER2-binding scFv linked to an Fc domain via a hinge comprising Ala-Ser (scFv-Fc). The Fc domain linked to the scFv includes Q347R, D399V, and F405T substitutions for heterodimerization and an S354C substitution for forming a disulfide bond with a Y349C substitution in SEQ ID NO:141 as described above. The scFv (SEQ ID NO:171) includes a heavy chain variable domain of pertuzumab connected to the N-terminus of a light chain variable domain of pertuzumab via a (G₄S)₄ linker (SEQ ID NO:143), the scFv represented as VL-(G₄S)₄-VH (“(G₄S)₄” is represented by SEQ ID NO:143). The heavy and the light variable domains of the scFv are also connected through a disulfide bridge between C100 of VL and C44 of VH, as a result of G100C and G44C substitutions in the VL and VH, respectively.

MGAH22 scFv
(SEQ ID NO: 171)
DIVMTQSHKFMSTSVGDRVSITCKASQDVNTAVAWYQQKPGHSPKLLIY
SASFRYTGVPDRFTGSRSGTDFTFTISSVQAEDLAVYYCQQHYTTPPTF
GCGTKVEIKRGGGGSGGGGSGGGGSGGGGSQVQLQQSGPELVKPGASLK
LSCTASGFNIKDTYIHWVKQRPEQCLEWIGRIYPTNGYTRYDPKFQDKA
TITADTSSNTAYLQVSRLTSEDTAVYYCSRWGGDGFYAMDYWGQGASVT
VSSA
MGAH22 scFv-Fc
(SEQ ID NO: 192)
DIVMTQSHKFMSTSVGDRVSITCKASQDVNTAVAWYQQKPGHSPKLLIY
SASFRYTGVPDRFTGSRSGTDFTFTISSVQAEDLAVYYCQQHYTTPPTF
GCGTKVEIKRGGGGSGGGGSGGGGSGGGGSQVQLQQSGPELVKPGASLK
LSCTASGFNIKDTYIHWVKQRPEQCLEWIGRIYPTNGYTRYDPKFQDKA
TITADTSSNTAYLQVSRLTSEDTAVYYCSRWGGDGFYAMDYWGQGASVT
VSSAASDKTHTCPPCPAPELLGGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPRVYTLPPCRDELTKN
QVSLTCLVKGFYPSDIAVEWESNGQPENNYKTTPPVLVSDGSFTLYSKL
TVDKSRWQQGNVFSCSVMHEALHNHYTQKSLSLSPG

Another TriNKET of the present disclosure is A49MI-F3′-TriNKET-MGAH22. A49MI-F3′-TriNKET-MGAH22 includes the same Her2-binding scFv (SEQ ID NO:171) as in A49-F3′-TriNKET-MGAH22 linked via a hinge comprising Ala-Ser to an Fc domain; and the same NKG2D-binding Fab fragment as in A49MI-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A49MI-F3′-KiH-TriNKET-MGAH22 includes three polypeptides, having the sequences of SEQ ID NO:192 (as in A49-F3′-TriNKET-MGAH22), SEQ ID NO:145 (as in A49MI-F3′-TriNKET-Trastuzumab), and SEQ ID NO: 142 (as in A49-F3′-TriNKET-Trastuzumab).

Another TriNKET of the present disclosure is A49-F3′-KiH-TriNKET-MGAH22. A49-F3′-KiH-TriNKET-MGAH22 includes the same Her2-binding scFv (SEQ ID NO: 171) as in A49-F3′-TriNKET-MGAH22 linked via a hinge comprising Ala-Ser to an Fc domain; and the same NKG2D-binding Fab fragment as in A49-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A49-F3′-KiH-TriNKET-MGAH22 includes three polypeptides, having the sequences of SEQ ID NO: 193, SEQ ID NO: 147 (as in A49-F3′-KiH-TriNKET-Trastuzumab), and SEQ ID NO:142 (as in A49-F3′-TriNKET-Trastuzumab).

SEQ ID NO: 193 represents the full sequence of the HER2-binding scFv (SEQ ID NO: 171) linked to an Fc domain via a hinge comprising Ala-Ser (scFv-Fc). The Fc domain linked to the scFv includes T366S, L368A, and Y407V substitutions for heterodimerization and an S354C substitution for forming a disulfide bond with a Y349C substitution in SEQ ID NO: 147 as described above.

MGAH22 scFv-Fc (KiH)
(SEQ ID NO: 193)
DIVMTQSHKFMSTSVGDRVSITCKASQDVNTAVAWYQQKPGHSPKLLIY
SASFRYTGVPDRFTGSRSGTDFTFTISSVQAEDLAVYYCQQHYTTPPTF
GCGTKVEIKRGGGGSGGGGSGGGGSGGGGSQVQLQQSGPELVKPGASLK
LSCTASGFNIKDTYIHWVKQRPEQCLEWIGRIYPTNGYTRYDPKFQDKA
TITADTSSNTAYLQVSRLTSEDTAVYYCSRWGGDGFYAMDYWGQGASVT
VSSAASDKTHTCPPCPAPELLGGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPQVCTLPPSRDELTKN
QVSLSCAVKGFYPSDIAVEWESNGQPENNYKTTPPVLDSDGSFFLVSKL
TVDKSRWQQGNVFSCSVMHEALHNHYTQKSLSLSPG

Another exemplary TriNKET of the present disclosure is A49MI-F3′-KiH-TriNKET-MGAH22. A49MI-F3′-KiH-TriNKET-MGAH22 includes the same Her2-binding scFv (SEQ ID NO:171) as in A49-F3′-TriNKET-MGAH22 linked via a hinge comprising Ala-Ser to an Fc domain; and the same NKG2D-binding Fab fragment as in A49MI-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A49MI-F3′-KiH-TriNKET-MGAH22 includes three polypeptides, having the sequences of SEQ ID NO: 193 (as in A49-F3′-KiH-TriNKET-MGAH22), SEQ ID NO: 194 (as in A49MI-F3′-KiH-TriNKET-Trastuzumab), and SEQ ID NO:142 (as in A49-F3′-TriNKET-Trastuzumab).

Another exemplary TriNKET of the present disclosure is A44-F3′-TriNKET-MGAH22. A44-F3′-TriNKET-MGAH22 includes the same Her2-binding scFv (SEQ ID NO: 171) as in A49-F3′-TriNKET-MGAH22 linked via a hinge comprising Ala-Ser to an Fc domain; and the same NKG2D-binding Fab fragment as in A44-F3′-TriNKET-Trastuzumab, the CH1 domain of which is connected to an Fc domain. The Fc domain linked to the scFv includes Q347R, D399V, and F405T substitutions, and the Fc domain linked to the Fab fragment includes K360E and K409W substitutions. A44-F3′-TriNKET-MGAH22 includes three polypeptides, having the sequences of SEQ ID NO: 192 (as in A49-F3′-TriNKET-MGAH22), SEQ ID NO:155 (as in A44-F3′-TriNKET-Trastuzumab), and SEQ ID NO:149 (as in A44-F3′-TriNKET-Trastuzumab).

Another exemplary TriNKET of the present disclosure is A44-F3′-KiH-TriNKET-MGAH22. A44-F3′-KiH-TriNKET-MGAH22 includes the same Her2-binding scFv (SEQ ID NO:171) as in A49-F3′-TriNKET-MGAH22 linked via a hinge comprising Ala-Ser to an Fc domain; and the same NKG2D-binding Fab fragment as in A44-F3′-TriNKET-Trastuzumab, the CH1 domain of which is connected to an Fc domain. The Fc domain linked to the scFv includes the “hole” substitutions of T366S, L368A, and Y407V, and the Fc domain linked to the Fab fragment includes the “knob” substitution of T366W. A44-F3′-KiH-TriNKET-MGAH22 includes three polypeptides, having the sequences of SEQ ID NO: 193 (as in A49-F3′-KiH-TriNKET-MGAH22), SEQ ID NO: 148 (as in A44-F3′-KiH-TriNKET-Trastuzumab), and SEQ ID NO:149 (as in A44-F3′-TriNKET-Trastuzumab).

In a certain embodiment, a TriNKET of the present disclosure is identical to one of the exemplary TriNKETs described above that includes the EW-RVT Fc mutations, except that the Fc domain linked to the NKG2D-binding Fab fragment comprises the substitutions of Q347R, D399V, and F405T, and the Fc domain linked to the HER2-binding scFv comprises matching substitutions K360E and K409W for forming a heterodimer. In certain embodiments, a TriNKET of the present disclosure is identical to one of the exemplary TriNKETs described above that includes the KiH Fc mutations, except that the Fc domain linked to the NKG2D-binding Fab fragment comprises the “hole” substitutions of T366S, L368A, and Y407V, and the Fc domain linked to the HER2-binding scFv comprises the “knob” substitution of T366W for forming a heterodimer.

In certain embodiments, a TriNKET of the present disclosure is identical to one of the exemplary TriNKETs described above, except that the Fc domain linked to the NKG2D-binding Fab fragment includes an S354C substitution in the CH3 domain, and the Fc domain linked to the HER2-binding scFv includes a matching Y349C substitution in the CH3 domain for forming a disulfide bond.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that comprise an Fc domain at its C-terminus. Accordingly, in some embodiments where the C-terminal residue of an amino acid sequence of a polypeptide (e.g., an Fc domain sequence) is K, a corresponding amino acid sequence with the K removed is also contemplated herein.

The multi-specific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins,

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multi-specific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of the Multi-Specific Proteins

In certain embodiments, a multi-specific binding protein of the present disclosure, e.g., A49-F3′-TriNKET-Trastuzumab, which include an NKG2D-binding Fab fragment and a HER2-binding scFv domain, bind to cells expressing low levels of HER2 at a level higher than a monoclonal antibody having the same HER2-binding domain. For example, the multi-specific binding proteins that include an NKG2D-binding Fab domain and a HER2-binding svFv domain derived from trastuzumab, e.g., A49-F3′-TriNKET-Trastuzumab, can bind to low-HER2 expressing cells at a level higher than trastuzumab.

Moreover, the multi-specific binding proteins described herein are more effective in reducing tumor growth and killing cancer cells. For example, a multi-specific binding protein of the present disclosure that targets HER2-expressing tumor/cancer cells is more effective than trastuzumab. A TriNKET of the present disclosure A49-F3′-TriNKET-Trastuzumab (comprising an HER2-binding scFv (SEQ ID NO:139) linked to an Fc domain via a hinge comprising Ala-Ser (scFv-Fc represented by SEQ ID NO:140); and an NKG2D-binding Fab fragment including a heavy chain portion comprising a heavy chain variable domain of ADI-27749 (A49) (SEQ ID NO:94) and a CH1 domain, and a light chain portion comprising a light chain variable domain (SEQ ID NO:98) and a light chain constant domain, where the heavy chain variable domain is connected to the CH1, and the CH1 domain is connected to the Fc domain (heavy chain portion represented as VH-CH1-Fc, amino acid sequence set forth in SEQ ID NO:141)) is effective in promoting NK-mediated cell lysis of a human cancer cell line with low level of HER2 expression (HER2+), while trastuzumab shows little activity against this cell line. Moreover, A49-F3′-TriNKET-Trastuzumab has superior NK-mediated cell lysis of a human cancer cell line with higher expression than the HER2+ cell line (HER2++) compared to trastuzumab. And even against a human cancer cell line with the highest level of HER2 expression (compared to HER+ and HER2++ cell lines) (HER2+++), A49-F3′-TriNKET-Trastuzumab has superior NK-mediated cell lysis compared to trastuzumab.

In some embodiments, the multi-specific binding proteins described herein including an NKG2D-binding domain (e.g., A49-F3′-TriNKET-Trastuzumab, A49MI-F3′-TriNKET-Trastuzumab, A49-F3′-KiH-TriNKET-Trastuzumab, A44-F3′-TriNKET-Trastuzumab) delay progression of the tumor more effectively than monoclonal antibodies that include the same tumor antigen-binding domain. In some embodiments, the multi-specific binding proteins including an NKG2D-binding domain (e.g., A49-F3′-TriNKET-Trastuzumab, A49MI-F3′-TriNKET-Trastuzumab, A49-F3′-KiH-TriNKET-Trastuzumab, A44-F3′-TriNKET-Trastuzumab) are more effective against cancer metastases than monoclonal antibodies that include the same tumor antigen-binding domain.

The multi-specific binding proteins described herein including an NKG2D-binding domain (e.g., A49-F3′-TriNKET-Trastuzumab, A49MI-F3′-TriNKET-Trastuzumab, A49-F3′-KiH-TriNKET-Trastuzumab, A44-F3′-TriNKET-Trastuzumab) bind to non-cancerous human cells (e.g., human cardiomyocytes) to a similar extent as binding to HER2++ cancer cells (medium level expression). However, despite the comparable binding, the multi-specific binding proteins do not induce NK-mediated killing of healthy non-cancerous human cells (e.g., human cardiomyocytes).

The multi-specific binding proteins described herein including an NKG2D-binding domain (e.g., A49-F3′-TriNKET-Trastuzumab, A49MI-F3′-TriNKET-Trastuzumab, A49-F3′-KiH-TriNKET-Trastuzumab, A44-F3′-TriNKET-Trastuzumab) trigger CD8+ T cell lysis of Tumor-Associated Antigen positive (TAA+) tumor cells. For example, A49-F3′-TriNKET-Trastuzumab enhances the cytotoxic activity of human primary CD8+ T cells after culture with IL-15 in a dose-dependent manner (FIG. 13A). A49-F3′-TriNKET-Trastuzumab also enhances the cytotoxic activity of human primary CD8+ T cells after culture with IL-2 (FIG. 13B). In contrast, anti-HER2 monoclonal antibodies margetuximab or trastuzumab does not have similar effects. Margetuximab (also called MGAH22) is an Fc-optimized monoclonal antibody that binds HER2. The heavy chain variable domain of margetuximab is represented by SEQ ID NO: 130, and the light chain variable domain of margetuximab is represented by SEQ ID NO: 134. Margetuximab includes F243L, R292P, Y300L, and P396L substitutions in the Fc domain, which are designed to be ADCC enhancing mutations. The heavy chain and light chain sequences are provided in SEQ ID NO:151 and SEQ ID NO:153, respectively. The F243L, R292P, Y300L, and P396L substitutions are bold-underlined.

MGAH22 heavy chain
(SEQ ID NO: 151)
QVQLQQSGPELVKPGASLKLSCTASGFNIKDTYIHWVKQRPEQGLEWIG
RIYPTNGYTRYDPKFQDKATITADTSSNTAYLQVSRLTSEDTAVYYCSR
WGGDGFYAMDYWGQGASVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELVGGPSVFL
LPPKPKDTLMISRTPEVTCVVVDVSHEDPEVKFNWYVDGVEVHNAKTKP
PEEQYNSTLRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPLVLDSDGSFFLYSKLTVDKSRWQQGNVFSCSVMHEALHNHYTQ
KSLSLSPGK
MGAH22 light chain
(SEQ ID NO: 153)
DIVMTQSHKFMSTSVGDRVSITCKASQDVNTAVAWYQQKPGHSPKLLIY
SASFRYTGVPDRFTGSRSGTDFTFTISSVQAEDLAVYYCQQHYTTPPTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

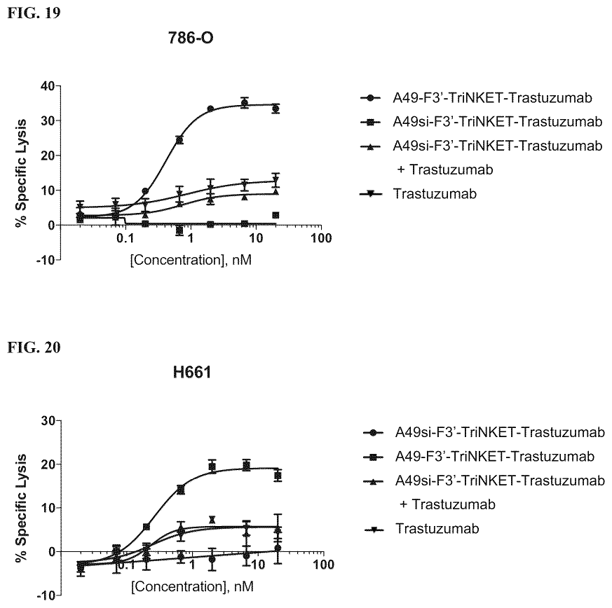

Compared to several HER2-targeted TriNKETs, A49-F3′-TriNKET-Trastuzumab shows weak binding to cells expressing NKG2D. The multi-specific binding proteins described herein including an NKG2D-binding domain (e.g., A49-F3′-TriNKET-Trastuzumab) exhibit a significant advantage in potency and maximum lysis of target cells compared to the combination of Fc-silent TriNKET (“A49si-F3′-TriNKET-Trastuzumab”; the amino acid sequence of the constant region has L234A, L235A, and P329G (LALAPG) mutations, which reduce effector functions of the Fc), and trastuzumab, when the target cells were HER2+ (786-O cells) (FIG. 19) or HER2++ (H661 cells) (FIG. 20), suggesting A49-F3′-TriNKET-Trastuzumab can mediate robust effector cell dependent killing of cancer cells expressing HER2.

Accordingly, compared to monoclonal antibodies, the multi-specific binding proteins described herein (e.g., A49-F3′-TriNKET-Trastuzumab) are advantageous in treating HER2-expressing cancers.

### III. Therapeutic Applications

The invention provides methods for treating cancer using a multi-specific binding protein described herein and/or a pharmaceutical composition described herein. The methods may be used to treat a variety of cancers which express HER2 by administering to a patient in need thereof a therapeutically effective amount of a multi-specific binding protein described herein.

The therapeutic method can be characterized according to the cancer to be treated. For example, in certain embodiments, the cancer is breast, ovarian, esophageal, bladder or gastric cancer, salivary duct carcinoma, salivary duct carcinomas, adenocarcinoma of the lung or aggressive forms of uterine cancer, such as uterine serous endometrial carcinoma.

In certain other embodiments, the cancer is brain cancer, breast cancer, cervical cancer, colon cancer, colorectal cancer, endometrial cancer, esophageal cancer, leukemia, lung cancer, liver cancer, melanoma, ovarian cancer, pancreatic cancer, rectal cancer, renal cancer, stomach cancer, testicular cancer, or uterine cancer. In yet other embodiments, the cancer is a squamous cell carcinoma, adenocarcinoma, small cell carcinoma, melanoma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well-differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

In certain other embodiments, the cancer is breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, lung squamous, mesothelioma, liver cancer, sarcoma, and gall bladder cancer.

The cancer to be treated can be characterized according to the presence of a particular antigen expressed on the surface of the cancer cell. In certain embodiments, the cancer cell can express one or more of the following in addition to HER2: CD2, CD19, CD20, CD30, CD38, CD40, CD52, CD70, EGFR/ERBB1, IGF1R, HER3/ERBB3, HER4/ERBB4, MUC1, cMET, SLAMF7, PSCA, MICA, MICB, TRAILR1, TRAILR2, MAGE-A3, B7.1, B7.2, CTLA4, and PD1.

### IV. Combination Therapy

Another aspect of the invention provides for combination therapy. A multi-specific binding protein described herein can be used in combination with additional therapeutic agents to treat cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma (IFN-γ), colony stimulating factor-1, colony stimulating factor-2, denileukin diftitox, interleukin-2, luteinizing hormone releasing factor and variations of the aforementioned agents that may exhibit differential binding to its cognate receptor, or increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T lymphocyte-associated antigen 4 (CTLA4), (ii) programmed cell death protein 1 (PD1), (iii) PDL1, (iv) LAG3, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in 12 to 60 vials. In certain embodiments, the formulation may be freeze-dried and 45 mg of the freeze-dried formulation may be contained in one vial. In certain embodiments, the about 40 mg—about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

The protein could exist in a liquid aqueous pharmaceutical formulation including a therapeutically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes about 1 to 1.5 mg/mL of citric acid, about 0.25 to 0.5 mg/mL of sodium citrate, about 1.25 to 1.75 mg/mL of disodium phosphate dihydrate, about 0.7 to 1.1 mg/mL of sodium dihydrogen phosphate dihydrate, and about 6.0 to 6.4 mg/mL of sodium chloride. In certain embodiments, the pH of the formulation is adjusted with sodium hydroxide.

A polyol, which acts as a tonicifier and may stabilize the antibody, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to 15 mg/mL. In certain embodiments, the concentration of mannitol may be about 10 to 14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at about a 10 mg/ml concentration in either a USP/Ph Eur type I 50R vial closed with a rubber stopper and sealed with an aluminum crimp seal closure. The stopper may be made of elastomer complying with USP and Ph Eur. In certain embodiments vials may be filled with about 61.2 mL of the protein product solution in order to allow an extractable volume of about 60 mL. In certain embodiments, the liquid formulation may be diluted with about 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variant of peptides and proteins that may occur during fermentation, harvest/cell clarification, purification, drug substance/drug product storage and during sample analysis. Deamidation is the loss of NH₃ from a protein forming a succinimide intermediate that can undergo hydrolysis. The succinimide intermediate results in a 17 dalton mass decrease of the parent peptide. The subsequent hydrolysis results in an 18 dalton mass increase. Isolation of the succinimide intermediate is difficult due to instability under aqueous conditions. As such, deamidation is typically detectable as 1 dalton mass increase. Deamidation of an asparagine results in either aspartic or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain affect deamidation rates. Gly and Ser following an Asn in protein sequences results in a higher susceptibility to deamidation.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of about 10 mM to 200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The protein of the present disclosure could exist in a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product of the current disclosure is reconstituted with either Sterile Water for Injection, USP (SWFI) or about 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50 to 5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308:43-53, 2001; Steimer et al., Clinica Chimica Acta 308:33-41, 2001).

In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

The description above describes multiple aspects and embodiments of the invention. The patent application specifically contemplates all combinations and permutations of the aspects and embodiments.

### Examples

The invention now being generally described, will be more readily understood by reference to the following examples, which are included merely for purposes of illustration of certain aspects and embodiments of the present invention, and is not intended to limit the invention.

### Example 1-Primary Human NK Cell Cytotoxicity Assay

Peripheral blood mononuclear cells (PBMCs) were isolated from human peripheral blood buffy coats using density gradient centrifugation. Isolated PBMCs were washed and prepared for NK cell isolation. NK cells were isolated using a negative selection technique with magnetic beads, purity of isolated NK cells was typically >90% CD3-CD56+. Isolated NK cells were rested overnight, rested NK cells were used the following day in cytotoxicity assays.

### DELFIA Cytotoxicity Assay:

Human cancer cell lines expressing HER2 were harvested from culture, cells were washed with HBS, and were resuspended in growth media at 106/mL for labeling with BATDA reagent (Perkin Elmer AD0116). Manufacturer instructions were followed for labeling of the target cells. After labeling cells were washed 3× with HBS, and were resuspended at 0.5-1.0×10⁵/mL in culture media. To prepare the background wells an aliquot of the labeled cells was put aside, and the cells were spun out of the media. 100 μl of the media were carefully added to wells in triplicate to avoid disturbing the pelleted cells. 100 μl of BATDA labeled cells were added to each well of the 96-well plate. Wells were saved for spontaneous release from target cells, and wells were prepared for max lysis of target cells by addition of 1% Triton-X. Monoclonal antibodies or a TriNKET against HER2 (A49-F3′-TriNKET-Trastuzumab (comprising an HER2-binding scFv (SEQ ID NO:139) linked to an Fc domain via a hinge comprising Ala-Ser (scFv-Fc represented by SEQ ID NO:140); and an NKG2D-binding Fab fragment including a heavy chain portion comprising a heavy chain variable domain of ADI-27749 (A49) (SEQ ID NO:94) and a CH1 domain, and a light chain portion comprising a light chain variable domain (SEQ ID NO:98) and a light chain constant domain, where the heavy chain variable domain is connected to the CH1, and the CH1 domain is connected to the Fc domain (heavy chain portion represented as VH-CH1-Fc, amino acid sequence set forth in SEQ ID NO:141))) were diluted in culture media, and 50 μl of diluted mAb or the TriNKET were added to each well. Rested NK cells were harvested from culture, cells were washed, and were resuspended at 105-2.0×10⁶/mL in culture media depending on the desired E:T ratio. 50 μl of NK cells were added to each well of the plate to make a total of 200 μl culture volume. The plate was incubated at 37° C. with 5% CO2 for 2-3 hours before developing the assay.

After culturing for 2-3 hours, the plate was removed from the incubator and the cells were pelleted by centrifugation at 200g for 5 minutes. 20 μl of culture supernatant was transferred to a clean microplate provided from the manufacturer, 200 μl of room temperature europium solution was added to each well. The plate was protected from the light and incubated on a plate shaker at 250 rpm for 15 minutes. Plate was read using either Victor 3 or SpectraMax i3X instruments. % Specific lysis was calculated as follows: % Specific lysis=((Experimental release-Spontaneous release)/(Maximum release-Spontaneous release))*100%.

Long term human PBMC cytotoxicity assay:

SkBr-3 cells stably expressing NucLight Green were generated using IncuCyte NucLight Green Reagent (catalog #4475). NucLight Green expressing cells were selected in puromycin to obtain a homogenous population. SkBr-3-NucLight Green cells were maintained in growth media containing puromycin before use in assays. SkBr-3-NucLight Green target cells were prepared as follows for cytotoxicity assays.

NucLight Green expressing cells were harvested from culture, and were washed to remove residual selection antibiotic, cells were resuspended in fresh culture media and seeded into a 96 well flat bottom plate. The plate was placed in the IncuCyte S3 overnight to monitor cell attachment and growth. The next day human PBMCs were isolated using density gradient centrifugation, and A49-F3′-TriNKET-Trastuzumab or mAb dilutions were prepared in primary cell culture media. Diluted A49-F3′-TriNKET-Trastuzumab and mAbs were added to SkBr-3-NucLight Green cells, followed by freshly isolated PBMCs. The plate was then returned to the IncuCyte S3.

Image collection was setup on the IncuCyte S3. Images for the phase and green channels were collected every hour, with 2 images per well. Image analysis was done using the IncuCyte S3 software. Masks for the green channel were created to count the number of SkBr-3 tumor cells. Percent growth was calculated as follows: % Growth=((Green object count time X)/(green object count time zero))*100%.

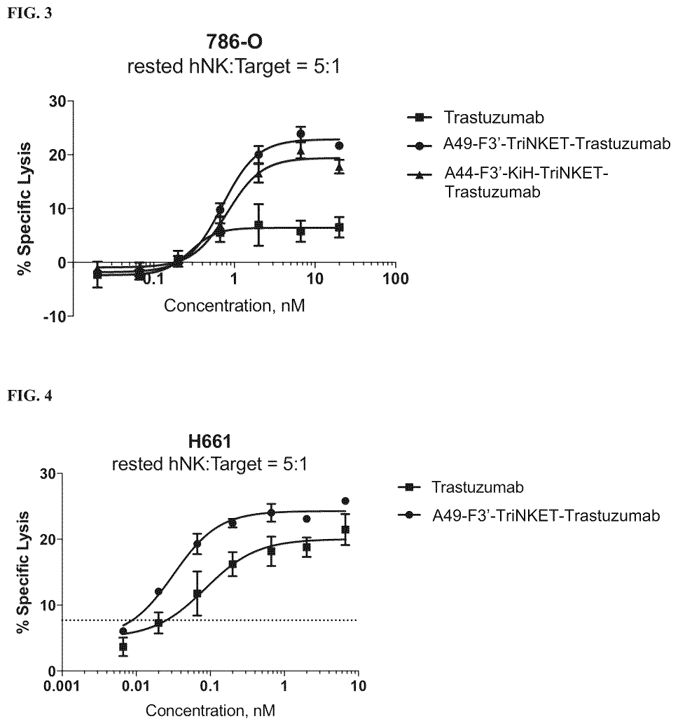

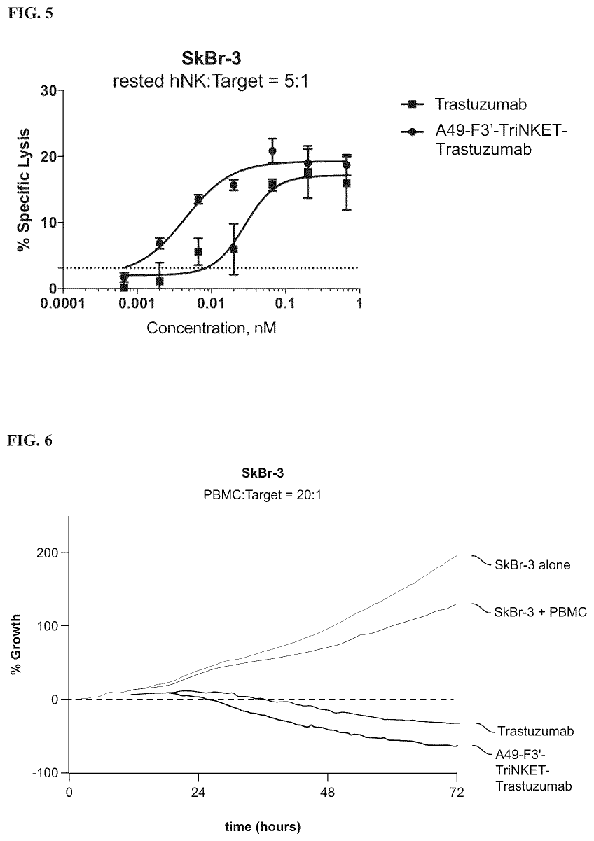

FIG. 3, FIG. 4, and FIG. 5 show TriNKET-mediated killing of three cell lines with different levels of HER2 expression. TriNKETs provided more potent, and higher maximal killing against all HER2-positive cells lines compared to the anti-HER2 monoclonal antibody trastuzumab.

FIG. 3 shows NK-mediated cell lysis of the HER2 1+ human cancer cell line 786-O. trastuzumab shows little activity against HER2 1+ cell lines, increasing specific lysis slightly higher than background killing. However, A49-F3′-TriNKET-Trastuzumab (comprising an HER2-binding scFv (SEQ ID NO:139) linked to an Fc domain via a hinge comprising Ala-Ser (scFv-Fc represented by SEQ ID NO:140); and an NKG2D-binding Fab fragment including a heavy chain portion comprising a heavy chain variable domain of ADI-27749 (A49) (SEQ ID NO:94) and a CH1 domain, and a light chain portion comprising a light chain variable domain (SEQ ID NO:98) and a light chain constant domain, where the heavy chain variable domain is connected to the CH1 domain, and the CH1 domain is connected to the Fc domain (heavy chain portion represented as VH-CH1-Fc, amino acid sequence set forth in SEQ ID NO:141)), and A44-F3′-KiH-TriNKET-Trastuzumab (comprising an HER2-binding scFv comprising SEQ ID NO:139, linked to an Fc domain via a hinge comprising Ala-Ser (scFv-Fc represented by SEQ ID NO:146), and an NKG2D-binding Fab fragment including a heavy chain portion comprising a heavy chain variable domain of ADI-27744 (A44) (SEQ ID NO:86) and a CH1 domain, and a light chain portion comprising a light chain variable domain (SEQ ID NO:90) and a light chain constant domain, where the heavy chain variable domain is connected to the CH1 domain, and the CH1 domain is connected to the Fc domain (heavy chain portion represented as VH-CH1-Fc, amino acid sequence set forth in SEQ ID NO:148)) were more effective at targeting 786-O cells, showing greater specific lysis than monoclonal antibody.

FIG. 4 demonstrates NK-mediated cell lysis of the human cancer cell line H661 with higher level of HER2 expression that the 786-O cell line (denoted as HER2++). Compared to the 786-O cell line, trastuzumab showed improved killing against higher levels of HER2 expressed on H661 target cells. Despite improved lysis mediated by trastuzumab, A49-F3′-TriNKET-Trastuzumab still showed superior lysis of H661 target cells for both potency and maximal killing.

FIG. 5 shows NK-mediated cell lysis of the cancer cell line SkBr-3 with the highest level of HER2 expression among the three cell lines tested (denoted as HER2+++). Trastuzumab showed increased potency against SkBr-3 cells compared to HER2+ and HER2++ cell lines, but A49-F3′-TriNKET-Trastuzumab still showed superior potency and maximal killing.

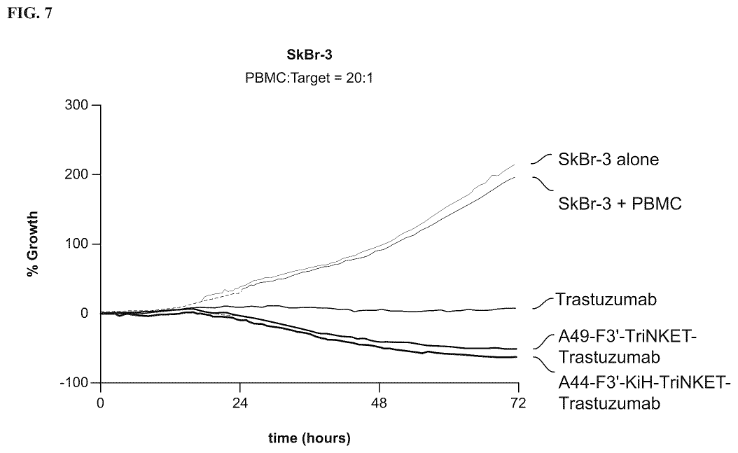

FIG. 6 and FIG. 7 show the effect of TriNKET or mAb on 72-hour co-cultures of human PBMCs and SkBr-3 HER2+ target cells. SkBr-3 cells proliferated about 3-fold over a 72-hour period when cultured without effector PBMCs. Donor variability was evident in effector PBMCs. When PBMCs were added to SkBr-3 cultures, growth of the target SkBr-3 cells was nominally reduced (FIG. 6). In another experiment, the addition of PBMC effector cells had negligible effect on SkBr-3 cell growth (FIG. 7). When trastuzumab was added to the co-culture, the effector PBMCs had an increased ability to lyse target SkBr-3 cells, indicated as a decrease in % growth in FIG. 6 and FIG. 7. When A49-F3′-TriNKET-Trastuzumab (FIG. 6 and FIG. 7) and A44-F3′-KiH-TriNKET-Trastuzumab (FIG. 7) were added to the co-culture, the effector PBMCs were even more effective in lysing SkBr-3 target cells compared to trastuzumab, resulting in a faster and more complete reduction in SkBr-3 cells.

### Example 2-Binding of TriNKETs in Human Whole Blood

100 μl of heparinized human whole blood was added to each tube/well. Trispecific-binding proteins (TriNKETs) or monoclonal Ab (mAb) was added directly into whole blood, and samples were incubated at room temperature for 20 minutes. For detection of unlabeled TriNKETs/mAbs, blood was washed 3× following incubation with TriNKET. Directly labeled immunophenotyping mAbs and secondary antibody specific to trastuzumab were added to samples. After a 20 minute incubation, 2 mL of 1× RBC lysis/fixation solution was added to each sample for 15 minutes at room temperature (RT), samples were then washed to remove red blood cells (RBCs). After washing, the samples were resuspended for FACS analysis.

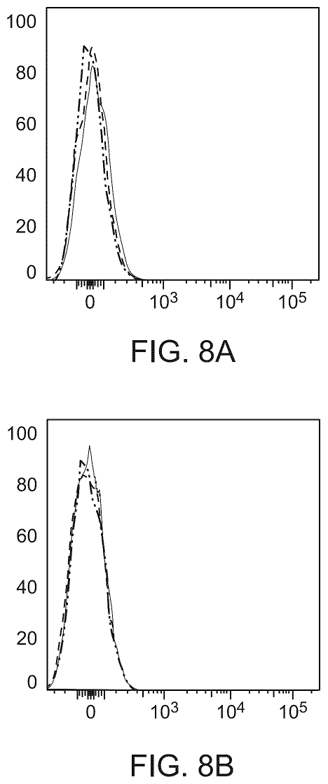

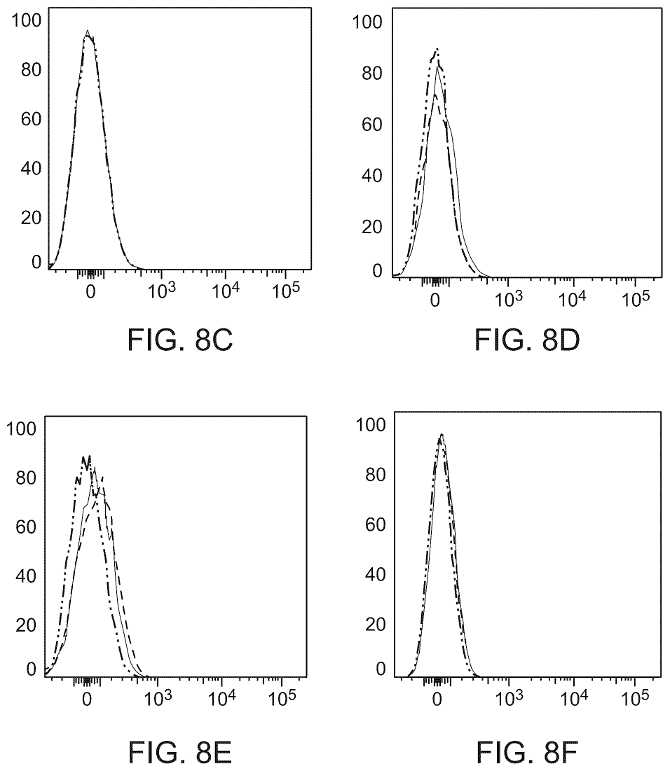

Binding of the A49-F3′-TriNKET-Trastuzumab was compared to trastuzumab and secondary antibody control samples. FIGS. 8A-8F show binding of A49-F3′-TriNKET-Trastuzumab (an NKG2D-binding domain from clone ADI-27749; and an HER2-binding scFv comprising SEQ ID NO:139, derived from trastuzumab monoclonal antibody) in whole human blood. A49-F3′-TriNKET-Trastuzumab binding in human whole blood was the same as trastuzumab. A49-F3′-TriNKET-Trastuzumab and trastuzumab demonstrated minimal binding to all populations of immune cells in blood. Small shifts were observed for trastuzumab and A49-F3′-TriNKET-Trastuzumab in both B cell and monocyte populations compared to secondary control samples. Binding observed on B cells and monocytes can likely be attributed to FcR interactions, rather than being Fab specific.

### Example 3-Assessment of A49-F3′-TriNKET-Trastuzumab Binding to Human Cardiomyocytes Versus Human Cancer Cells Expressing Different Levels of HER2

Human cardiomyocytes differentiated from induced pluripotent stem cells (Cellular Dynamics/Fuji Film), 786-O, H661 and SKBR3 cancer cells were used to evaluate binding of A49-F3′-TriNKET-Trastuzumab to these cells. The human renal cell carcinoma cell line 786-O expresses low levels of HER2, the human lung cancer cell line H661 expresses moderate levels of HER2, while the human breast cancer cell line SKBR3 expresses high levels of HER2. TriNKETs were diluted to 3.8e-4 to 100 μg/mL, and the dilutions were used as primary antibody stain. Binding of the TriNKET was detected using a fluorophore-conjugated anti-human IgG secondary antibody. Cells were analyzed by flow cytometry. Binding fluorescence intensity (MFI) to cells expressing HER2 was normalized to cells stained with a control (non-specific) TriNKET to obtain fold over background (FOB) values.

### Human PBMC Cytotoxicity Assay

PBMCs were isolated from human peripheral blood buffy coats using density gradient centrifugation. SKBR3 target cells were labeled with BacMam 3.0 NucLight Green (#4622) to allow for tracking of the target cells. The manufacturer's protocol was followed for labeling of SKBR3 target cells. Human cardiomyocytes were unlabeled. Monoclonal antibodies or TriNKETs were diluted into culture media. 50 μl of TriNKETs and human PBMCs were added to wells of a 96-well plate already containing target cells, 50 μl of complete culture media was added for a total of 200 μl culture volume.

Image collection was setup on the IncuCyte S3. Image analysis was done using the IncuCyte S3 software. Masks for the green channel was created to count the number of tumor cells. Confluency of cardiomyocytes in the phase channel was used to assess cell viability and calculate % killing.

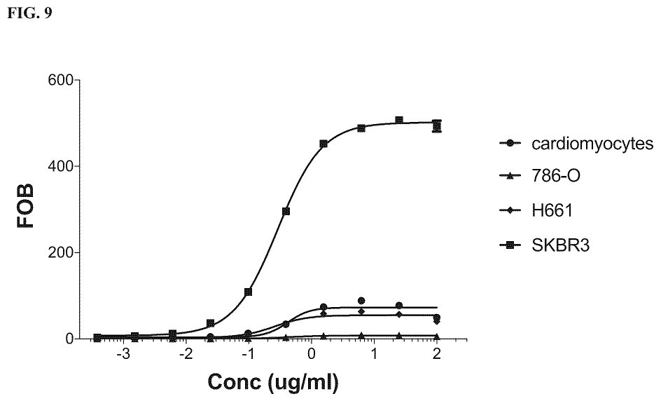

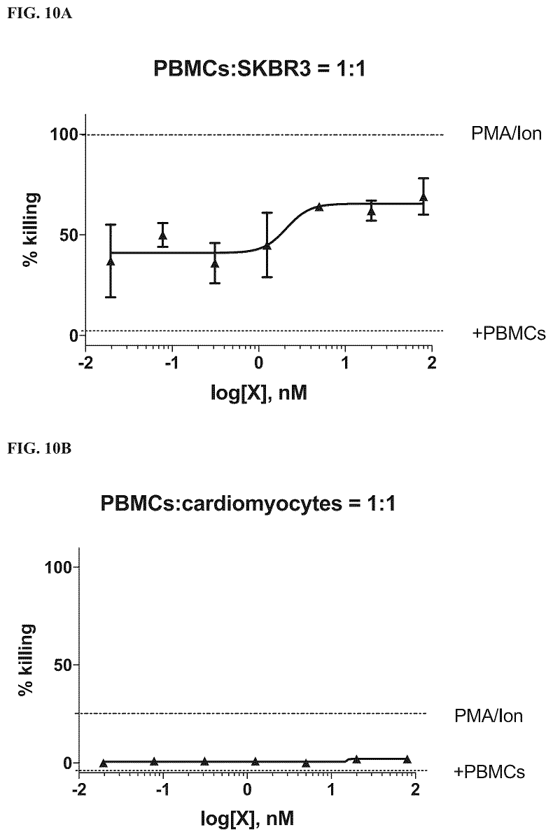

A49-F3′-TriNKET-Trastuzumab binds to human cardiomyocytes to similar extent as binding to H661 cells. FIG. 9 shows binding of A49-F3′-TriNKET-Trastuzumab to human cardiomyocytes, SKBR3, H661 and 786-O cancer cells, where the binding to cardiomyocytes is similar to H661 cells (medium HER2 surface expression levels).

### Primary Human PBMC Cytotoxicity Assay

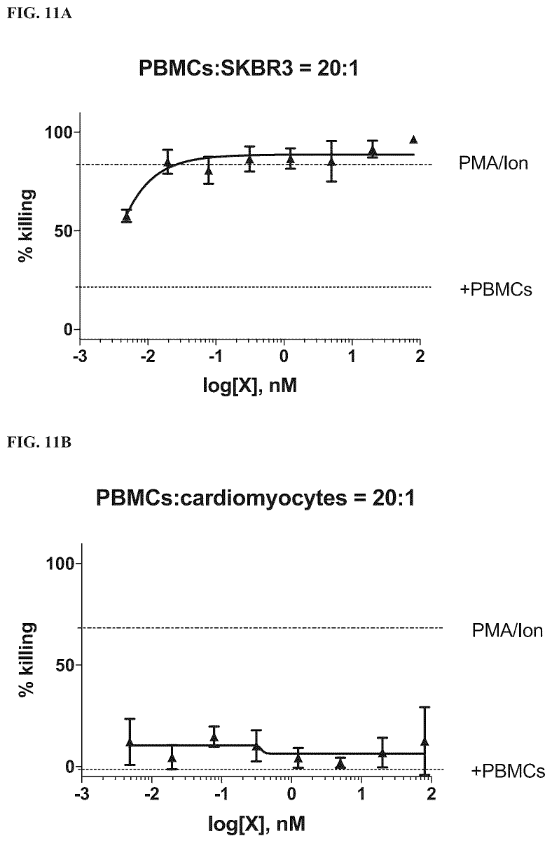

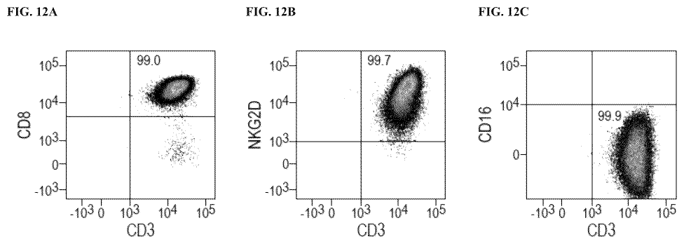

FIGS. 10A-10B and FIGS. 11A-11B show killing of SKBR3 cells by human PBMCs in the presence of A49-F3′-TriNKET-Trastuzumab, whereas viability of cardiomyocytes are minimally affected.

FIG. 10A shows A49-F3′-TriNKET-Trastuzumab-mediated human PBMC killing of SKBR3 cancer cells; FIG. 10B shows that A49-F3′-TriNKET-Trastuzumab did not kill non-malignant healthy cardiomyocytes after 3 days in co-culture at PBMC to target cell ratio (E:T) of 1:1.

FIG. 11A shows A49-F3′-TriNKET-Trastuzumab-mediated human PBMC killing of SKBR3 cancer cells; FIG. 11B shows that A49-F3′-TriNKET-Trastuzumab did not kill non-malignant healthy cardiomyocytes after 3 days in co-culture at E:T of 20:1.

### Example 4-TriNKETs Trigger CD8+ T Cell Lysis of TAA+ Tumor Cells

### Primary Human CD8 T Cell Cytotoxicity Assay: Primary Human CD8 Effector T Cell Generation

Human PBMCs were isolated from human peripheral blood buffy coats using density gradient centrifugation. Isolated PBMCs were stimulated with 1 μg/mL Concanavalin A (ConA) at 37° C. for 18 hours. Then ConA was removed and cultured with 25 unit/mL IL-2 at 37° C. for 4 days. CD8+ T cells were purified using a negative selection technique with magnetic beads, then cultured in media containing 25 unit/mL IL-2 or 10 ng/ml IL-15 at 37° C. for 8-10 days.

### Primary Human CD8 Effector T Cell Characterization

Human effector CD8+ T cells generated above were analyzed by flow cytometry for CD8+ T cell purity as well as NKG2D and CD16 expression. Cells were stained with fluorophore conjugated antibodies against CD3, CD8, NKG2D, CD16, and analyzed by flow cytometry.

### Short-Term CD8 Effector T Cell DELFIA Cytotoxicity Assay

Human cancer cell line SkBr-3 expressing a target of interest, HER2, was harvested from culture. Cells were washed and resuspended in growth media at 106/mL for labeling with BATDA reagent (Perkin Elmer AD0116). Manufacturer instructions were followed for labeling of the target cells. After labeling cells were washed three times with HBS, and were resuspended at 0.5×10⁵/mL in culture media. 100 μl of BATDA labeled cells were added to each well of the 96-well plate. Wells were saved for spontaneous release from target cells, and wells were prepared for max lysis of target cells by addition of 1% Triton-X.

Monoclonal antibodies, TriNKETs and controls were diluted in culture media; 50 μl of diluted mAb/TriNKET were added to each well. CD8 effector T cells were harvested from culture, washed, and resuspended at 5×10⁶/mL in culture media (E:T ratio=50:1). Then 50 μl of CD8 T cells were added to each well of the plate to make a total of 200 μl culture volume. The plate was incubated at 37° C. with 5% CO2 for 3.5 hours before developing the assay. After incubation, the plate was removed from the incubator and the cells were pelleted by centrifugation at 500 g for 5 minutes. Then 20 μl of culture supernatant were transferred to a clean microplate provided from the manufacturer, 200 μl of room temperature europium solution were added to each well. The plate was protected from the light and incubated on a plate shaker at 250 rpm for 15 minutes. The plate was read using either Victor 3 or SpectraMax i3X instruments. % Specific lysis was calculated as follows: % Specific lysis= ((Experimental release-Spontaneous release)/(Maximum release-Spontaneous release))*100%.

### Long-Term CD8 Effector T Cell Incucyte Cytotoxicity Assay

Human cancer cell line SkBr-3 expressing a target of interest, HER2, was labeled with BacMam 3.0 NucLight Green (#4622) to allow for tracking of the target cells. SkBr-3 target cells were harvested from culture, washed, resuspended in growth media, and plated at 5,000/well in a 96-well plate. The plate was incubated at 37° C. with 5% CO2 overnight. Monoclonal antibodies, TriNKETs and controls were diluted in culture media; 50 μl of diluted mAb or TriNKET were added to each well. CD8 effector T cells were harvested from culture, washed, and resuspended at 1×10⁶/mL in culture media (E:T ratio=10:1). Then 50 μl of CD8+ T cells were added to each well of the plate to make a total of 200 μl culture volume. The plate was incubated at 37° C. with 5% CO2 for up to 7 days. Image collection was setup on the IncuCyte S3. Images for the phase and green channels were collected every hour, with 2 images per well. Image analysis was done using the IncuCyte S3 software. Green object count/well was used to measure the number of live tumor cells.

### Characterization of CD8 T Effector Cells Used in Cytotoxicity Assay

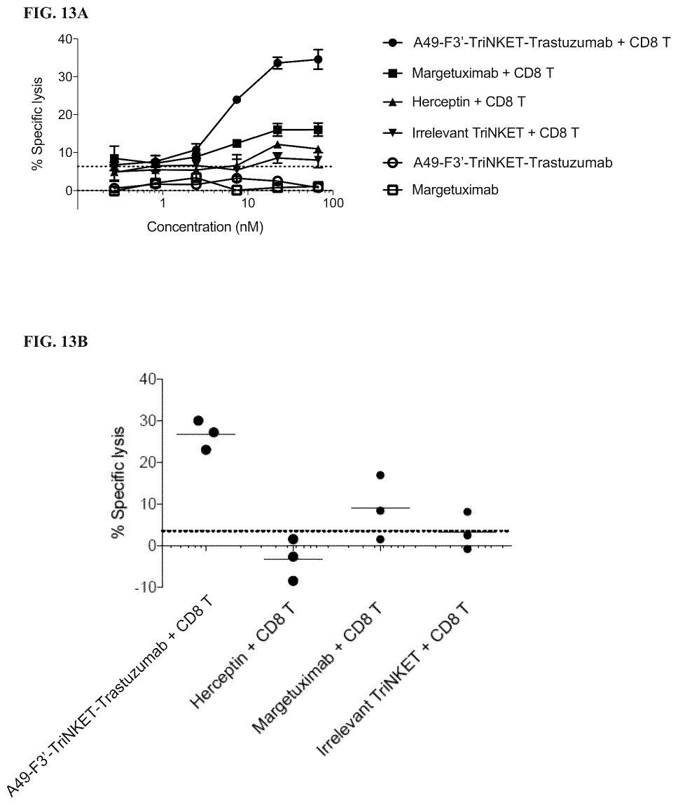

CD8+ T cells generated with conA stimulation and cultured with IL-15 were of high purity (99% of CD3+CD8+ cells), and all expressed NKG2D but not CD16 (FIGS. 12A-12C). Similar results are observed with CD8+ T cells generated with IL-2 culture.

### Short-Term CD8 Effector T Cell DELFIA Cytotoxicity Assay

The effect of A49-F3′-TriNKET-Trastuzumab on the cytotoxic activity of human primary CD8+ T cells after culture with IL-15 was assayed. A49-F3′-TriNKET-Trastuzumab enhanced the cytotoxic activity of human primary CD8+ T cells after culture with IL-15 in a dose-dependent manner (FIG. 13A). A49-F3′-TriNKET-Trastuzumab also enhanced the cytotoxic activity of human primary CD8+ T cells after culture with IL-2 (FIG. 13B). Margetuximab or Herceptin did not show the effects observed with A49-F3′-TriNKET-Trastuzumab.

### Long-Term CD8 Effector T Cell Incucyte Cytotoxicity Assay

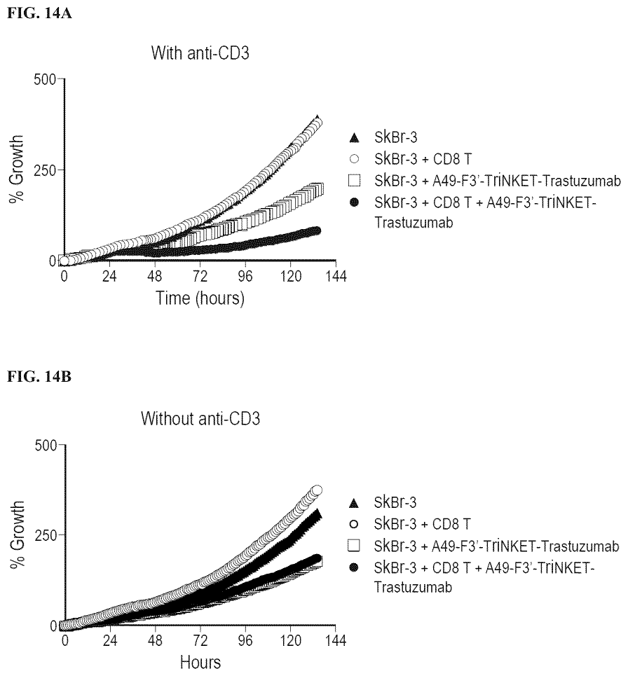

SkBr-3 cells cultured alone or co-cultured with CD8+ T cells proliferated in the culture (FIG. 14A). When the cells were treated with A49-F3′-TriNKET-Trastuzumab, the growth of SkBr-3 showed some inhibition due to HER2 signal blockade (FIG. 14A). In the presence of anti-CD3 antibody (mIgG1 antibody cross-linked with F (ab′) 2 to anti-mIgG1) (FIG. 14A), adding A49-F3′-TriNKET-Trastuzumab to the co-culture of SkBr-3 cells and CD8 T cells showed much more inhibition of tumor cell growth than A49-F3′-TriNKET-Trastuzumab alone, indicating that A49-F3′-TriNKET-Trastuzumab enhances CD8+ T cell cytotoxicity activity. When T cells were not activated by anti-CD3, the addition of CD8+ T cells did not further inhibit cell growth, indicating that the ability of A49-F3′-TriNKET-Trastuzumab to enhance CD8+ T cell cytotoxicity is dependent on T cell activation by anti-CD3 (FIG. 14B).

### Example 5-Assessment of TriNKET Binding to Cell Expressed Human Cancer Antigens TriNKET Binding Assay

Human cancer cell lines expressing HER2 were used to assess tumor antigen binding of a TriNKET (A49-F3′-TriNKET-Trastuzumab) and monoclonal antibodies. The human renal cell carcinoma cell line 786-O expressed low levels of HER2, the human lung cancer cell line NCI-H661 expressed moderate levels of HER2, and the human breast cancer cell line SkBr-3 expressed high levels of HER2. All three cell lines were used to assess their binding affinity to TriNKET. TriNKETs were diluted in a series of concentrations, and were incubated with the respective cells.

Binding of the TriNKET (A49-F3′-TriNKET-Trastuzumab) to the cells was detected using a fluorophore conjugated to an anti-human IgG secondary antibody, and the cells were analyzed by flow cytometry. The level of binding at each concentration of the TriNKET (A49-F3′-TriNKET-Trastuzumab) was calculated as a percentage value of median fluorescence intensity (MFI) of the cells relative to the maximum MFI observed with the cells incubated with 670 nM of TriNKET. Alternatively, the level of binding at each concentration of TriNKET was calculated as a fold over background (FOB) value of MFI of the cells relative to the background MFI observed with the cells incubated with the secondary antibody only. Trastuzumab was used as a control in place of TriNKET in each experiment.

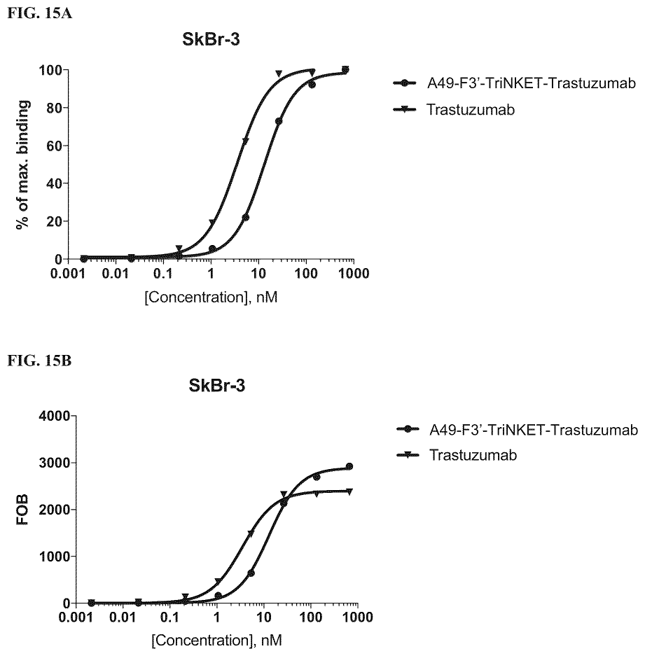

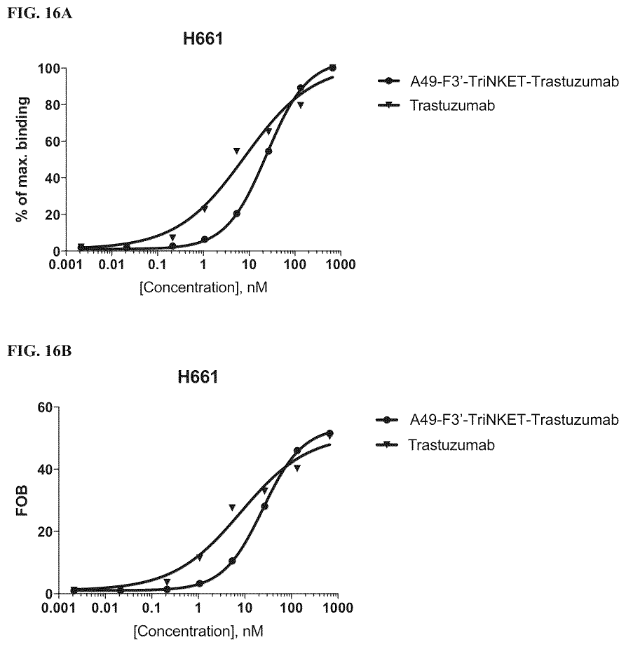

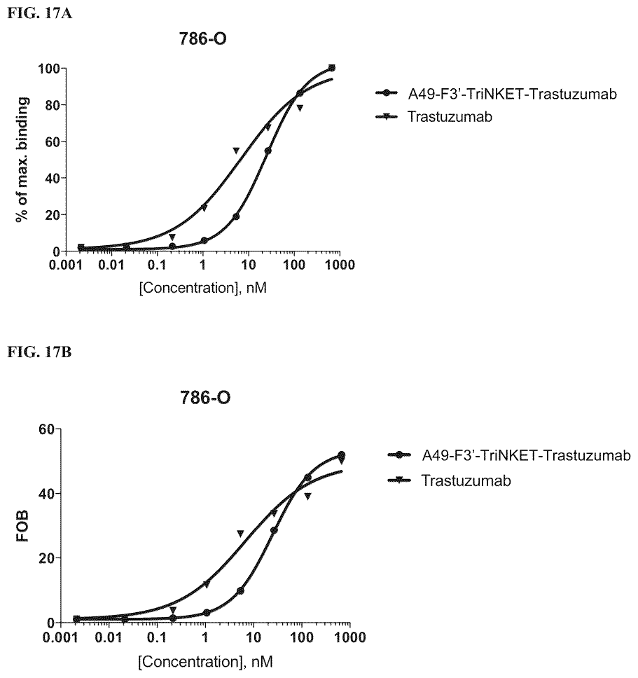

As shown in FIGS. 15A-15B, 16A-16B, and 17A-17B, the TriNKET (A49-F3′-TriNKET-Trastuzumab) and trastuzumab exhibited the most potent binding to the SkBr-3 cells (FIG. 15A), which had a high expression level of HER2 (HER+++). The binding affinity of the HER2-targeted TriNKET and trastuzumab to the NCI-H661, which had a moderate expression level of HER2 (HER2++), was similar (FIG. 16A) to but slightly higher than the binding affinity to the 786-O cells (FIG. 17A), which had a low expression level of HER2 (HER2+). This result suggested that the affinity of the HER2-targeted TriNKET and trastuzumab to HER2-expressing cells generally correlated with the expression level of HER2 on the cells.

Trastuzumab showed a higher binding affinity than the TriNKET (A49-F3′-TriNKET-Trastuzumab) with each of the three cell lines (SkBr-3 (FIG. 15A), NCI-H661 (FIG. 16A), and 786-O (FIG. 17A)). However, when expressed as fold over background (FOB) values, the maximum binding of the TriNKET to each of the three cell lines was greater than the maximum binding of trastuzumab (SkBr-3 (FIGS. 15B), NCI-H661 (FIG. 16B), and 786-O (FIG. 17B)). The difference was especially significant with the SkBr-3 cells, which had a high HER2 expression level (FIG. 15B).

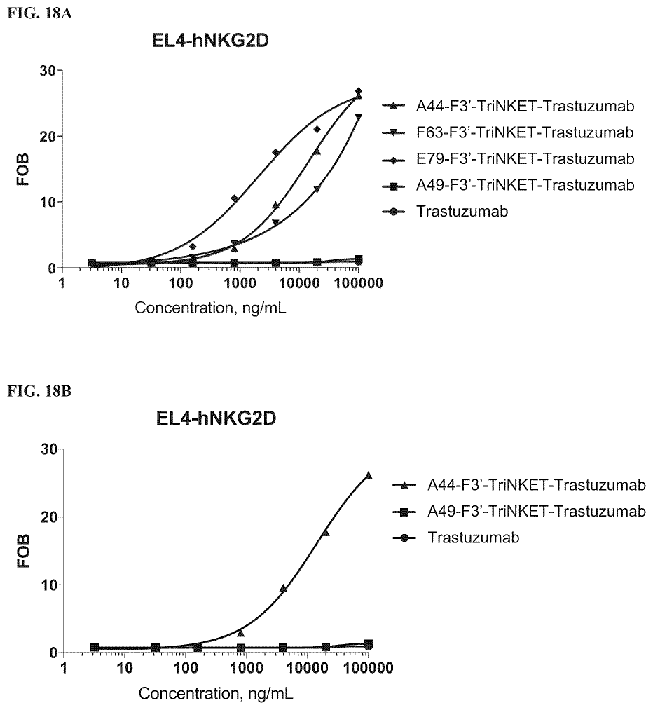

The affinity of TriNKETs to EL4 cells that express NKG2D was measured by a similar method. As shown in FIGS. 18A-18B, different NKG2D targeting domains used in the TriNKET resulted in different levels of binding to NKG2D expressed on EL4 cells. TriNKETs of clones A44, F63, and E79 bound to NKG2D strongly (FIG. 18A and FIG. 18B), whereas a TriNKET of clone A49 bound to NKG2D weakly (FIG. 18A and FIG. 18B).

### Primary Human NK Cell Cytotoxicity Assay

PBMCs were isolated from human peripheral blood buffy coats using density gradient centrifugation and were washed. NK cells were isolated from the PBMCs using a negative selection technique with magnetic beads. Typically, with this technique, more than 90% of the harvested cells were CD3-CD56+. The isolated NK cells were rested overnight. Rested NK cells were used the following day in cytotoxicity assays.

### DELFIA Cytotoxicity Assay

Human cancer cell lines expressing a target of interest were harvested from culture. The cells were washed with HBS, and were resuspended in growth media at 10⁶ cells/mL for labeling with BATDA reagent (Perkin Elmer AD0116). Manufacturer instructions were followed for labeling of the target cells. After labeling, the cells were washed three times with HBS, and were resuspended at 0.5-1.0×10⁵/mL in culture media. 100 μl of BATDA labeled cells were added to each well of the 96-well plate. Trastuzumab and A49-F3′-TriNKET-Trastuzumab was diluted in culture media, and 50 μl of diluted trastuzumab and A49-F3′-TriNKET-Trastuzumab were added, respectively, to each of their corresponding wells for the experiment. Rested and/or activated NK cells were harvested from culture. The cells were washed and resuspended at 105-2.0×10⁶/mL in culture media depending on the desired E:T ratio. 50 μl of NK cells were added to each well of the plate to make a total of 200 μl culture volume. To measure the spontaneous release of the BATDA hydrolysis product (e.g., due to spontaneous cell death), no NK cell, mAb, or TriNKET was added to the target cells. To measure the maximum release of the BATDA hydrolysis product, the target cells were lysed by addition of 1% Triton-X. The plate was incubated at 37° C. with 5% CO2 for 2-3 hours.

After culturing for 2-3 hours, the plate was removed from the incubator and the cells were pelleted by centrifugation at 200g for 5 minutes. 20 μl of culture supernatant were transferred to a clean microplate provided from the manufacturer, and 200 μl of room temperature europium solution were added to each well. The plate was protected from light and incubated on a plate shaker at 250 rpm for 15 minutes. Fluorescence levels were read using either Victor 3 or SpectraMax i3X instruments.

The percentage of specific lysis was calculated as: % Specific lysis= (Experimental release-Spontaneous release)/(Maximum release-Spontaneous release)*100%

To mimic the binding properties of TriNKETs on separate molecules, bispecific antibody engaging NKG2D and HER2 was combined with trastuzumab, and compared to the TriNKET containing all three binding domains on one molecule. Accordingly, 786-O and H661 target cells were incubated with isolated NK cells in the presence of HER2-targeted TriNKET, trastuzumab, Fc-silent TriNKET (comprising SEQ ID NO:156 and SEQ ID NO: 157, both of which include L234A, L235A, and P329G (LALAPG) substitutions in the Fc domain (shown within a third-bracket [ ])), or the combination of Fc-silent TriNKET and trastuzumab. Remarkably, TriNKET exhibited a significant advantage in potency and maximum lysis of target cells compared to the combination of Fc-silent TriNKET and trastuzumab, when the target cells were 786-O cells (FIG. 19) or H661 cells (FIG. 20).

scFv-Fc
(Includes L234A, L235A, and P329G (LALAPG)
substitutions (shown within a third-bracket [ ]))
(SEQ ID NO: 156)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GCGTKVEIKGGGGSGGGGSGGGGSGGGGSEVQLVESGGGLVQPGGSLRL
SCAASGFNIKDTYIHWVRQAPGKCLEWVARIYPTNGYTRYADSVKGRFT
ISADTSKNTAYLQMNSLRAEDTAVYYCSRWGGDGFYAMDYWGQGTLVTV
SSASDKTHTCPPCPAPE[AA]GGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKAL[G]APIEKTISKAKGQPREPRVYTLPPCRDELT
KNQVSLTCLVKGFYPSDIAVEWESNGQPENNYKTTPPVLVSDGSFTLYS
KLTVDKSRWQQGNVFSCSVMHEALHNHYTQKSLSLSPG
Whole chain VH-CH1-Fc
(Includes L234A, L235A, and P329G (LALAPG)
substitutions (shown within a third-bracket [ ]))
(SEQ ID NO: 157)
EVQLVESGGGLVKPGGSLRLSCAASGFTFSSYSMNWVRQAPGKGLEWVS
SISSSSSYIYYADSVKGRFTISRDNAKNSLYLQMNSLRAEDTAVYYCAR
GAPMGAAAGWFDPWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPE[AA]GGP
SVFLFPPKPKDTLMISRTPEVTCVVVDVSHEDPEVKFNWYVDGVEVHNA
KTKPREEQYNSTYRVVSVLTVLHQDWLNGKEYKCKVSNKAL[G]APIEK
TISKAKGQPREPQVCTLPPSRDELTENQVSLTCLVKGFYPSDIAVEWES
NGQPENNYKTTPPVLDSDGSFFLYSWLTVDKSRWQQGNVFSCSVMHEAL
HNHYTQKSLSLSPG

### Example 6-Variants of ADI-27749 and TriNKETs Containing the Variants

As described above, ADI-27749 (A49) contains, inter alia, a heavy chain CDR3 having the amino acid sequence of GAPMGAAAGWFDP (SEQ ID NO:169). The Met at position 102 of SEQ ID NO:94 (i.e., at position 4 of this CDR3 sequence) may be replaced by Gln, Leu, Ile, Phe, or Val, thereby generating NKG2D-binding sites A49MQ, A49ML, A49MI, A49MF, and A49MV, respectively, having the corresponding heavy chain variable region, light chain variable region, and CDR sequences provided in Table 1.

Binding of A49-F3′-TriNKET-Trastuzumab (“TriNKET A”) and a mutant form of TriNKET A having a substitution of Ile for the Met (“TriNKET A*”) to a fusion protein of human NKG2D and murine Fc (“mFc-hNKG2D”) was characterized by surface plasmon resonance (SPR) at 37° C. Steady state affinity fit was utilized to obtain the equilibrium affinity data. The equilibrium affinity constants were calculated, and data from two independent experiments for TriNKET A*and the independent experiments for TriNKET A were averaged.

TABLE 12

| | | Steady |
|---|---|---|
| | | State |
| | | Affinity K_{D} |
| Capture | Analyte | (M) |
| mFc- | TriNKET A* | 5.09 × 10⁻⁷ |
| hNKG2D | | |
| mFc- | TriNKET A* | 4.54 × 10⁻⁷ |
| hNKG2D | | |
| Average | | 4.81 × 10⁻⁷ |
| mFc- | TriNKET A | 3.70 × 10⁻⁷ |
| hNKG2D | | |
| mFc- | TriNKET A | 3.28 × 10⁻⁷ |
| hNKG2D | | |
| mFc- | TriNKET A | 3.13 × 10⁻⁷ |
| hNKG2D | | |
| Average ± stdev | | (3.37 ± 0.30) × 10⁻⁷ |

As shown in Table 12, the equilibrium affinity constant (K_{D}) obtained from the affinity fit was very similar between the replicates, which suggested a high confidence in the measured parameters. The K_{D} values indicated that the M102 variant has less than 2-fold reduced affinity for human NKG2D compared to TriNKET A. The K_{D} for TriNKET A*was (4.81±0.39)×10⁻⁷ M, while the K_{D} for TriNKET A was (3.37±0.30)×10⁻⁷ M (calculated from the affinity fit). Thesee K_{D} values suggested that the M102 mutation had only a minor effect on the binding of an A49-containing TriNKET to human NKG2D.

Additionally, the effect of the M102 mutation on the potency of TriNKETs was assessed in a cytotoxicity assay. Briefly, KHYG-1 cells expressing the high-affinity variant of CD16a (158V) were generated through retroviral transduction. Following transduction, cells were selected in puromycin-containing growth media to generate a selected population of KHYG-1-CD16V cells. The selected population was maintained in media containing 10 ng/ml human IL-2. To prepare the KHYG-1-CD16V cells for use as effectors in cytotoxicity assays, the cells were harvested from culture, pelleted, washed three times in culture media without IL-2, and resuspended in culture media without IL-2 and rested for 24 hours.

To measure the activity of TriNKET A and TriNKET A*, human cancer cell line SKBR-3 expressing the tumor antigen HER2 were selected as target cells. SKBR-3 expressing HER2 were harvested from culture. The cells were washed with Hepes Buffered Saline (HBS), and were resuspended in growth media at 10⁶ cells/mL for labeling with BATDA (hydrophobic esterified form of TDA (bis(acetoxymethyl) 2,2′: 6′,2″-terpyridine-6,6″-dicarboxylate) reagent (Perkin Elmer C136-100) (BATDA diffuses through the cell membrane of viable cells, and is hydrolyzed by intracellular esterases resulting in accumulation of membrane permeable TDA inside target cells. After incubation of the target cells with the effector cells, the TDA released from lysed cells into the supernatant is chelated with Eu3+, and the NK cell activity is quantified by measuring the intense fluorescence of the EuTDA chelate formed. (See see Blomberg et al. J. Immunol. Methods (1996) 193 (2): 199-206)). Manufacturer instructions were followed for labeling of the target cells. After labeling, the cells were washed three times with HBS and were resuspended at 0.5×10⁵ cells/mL in culture media. 100 μl of BATDA labeled cells were added to each well of a 96-well plate.

TriNKETs were serially diluted in culture media, and 50 μl of a diluted TriNKET were added to each well. Rested NK cells were harvested from culture, washed, and resuspended at 1.0×10⁶ cells/mL in culture media. 50 μl of NK cells were added to each well of the plate to attain a desired E:T ratio of 10:1 and to make a total of 200 μl culture volume in each well. The plate was incubated at 37° C. with 5% CO2 for 2-3 hours.

After culturing, the plate was removed from the incubator, and the cells were pelleted by centrifugation at 200×g for 5 minutes. 20 μl of culture supernatant were transferred to a clean microplate provided from the manufacturer. Supernatant from the labeled cells incubated alone without NK cells was used to measure spontaneous release of fluorescence enhancing ligand 2,2′: 6′,2″-terpyridine-6,6″-dicarboxylic acid (TDA) (see Blomberg et al. J. Immunol. Methods (1996) 193 (2): 199-206). Supernatant from labeled cells incubated with 1% Triton-X was used to measure maximum lysis of the target cells. Supernatant from the labeled cells prior to the 2-3 hours of incubation was used to measure the background and for quality control purposes.

200 μl of room temperature europium solution (Perkin Elmer C135-100) was added to each well containing culture supernatant. The plate was protected from light and incubated on a plate shaker at 250 rpm for 15 minutes. Fluorescence was measured using a SpectraMax i3X instrument. The fluorescent levels represented lysis of the target cells. The values of % specific lysis were calculated as: % specific lysis=((Experimental release-Spontaneous release)/(Maximum release-Spontaneous release))×100%.

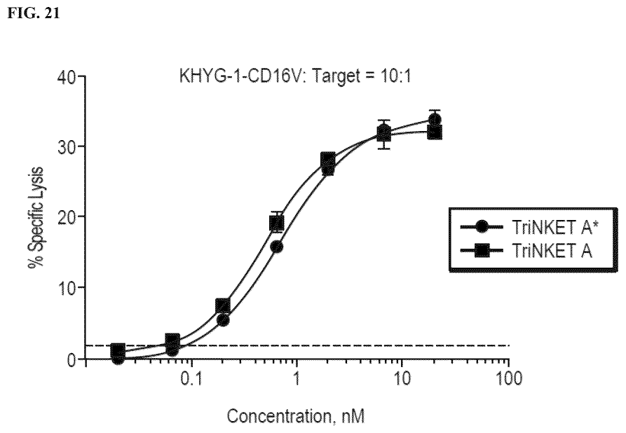

The % specific lysis values were plotted in FIG. 21, and the EC50 and maximum % specific lysis values are summarized in Table 13.

TABLE 13

| Protein | EC₅₀ (nM) | Max lysis (%) |
|---|---|---|
| TriNKET A | 0.39 | 35 |
| TriNKET A* | 0.65 | 36 |

The EC₅₀ was less than 2-fold increased and maximum % specific lysis values of TriNKET A*was identical to that of TriNKET A, suggesting that the M102 mutation did not have a substantial affect on the biological activity of TriNKET A.

### Numbered Embodiments

Embodiments disclosed herein include embodiments P1 to P49, as provided in the numbered embodiments of the disclosure:

Embodiment P1: A protein comprising: (a) a first antigen-binding site comprising an Fab fragment that binds NKG2D; (b) a second antigen-binding site comprising a single-chain variable fragment (scFv) that binds HER2; and (c) an antibody Fc domain or a portion thereof sufficient to bind CD16, or a third antigen-binding site that binds CD16.

Embodiment P2: The protein of embodiment P1, wherein the scFv is linked to the antibody Fc domain or a portion thereof sufficient to bind CD16, or the third antigen-binding site that binds CD16, via a hinge comprising Ala-Ser, wherein the scFv comprises a heavy chain variable domain and a light chain variable domain.

Embodiment P3: The protein according to embodiment P2, wherein the scFv is linked to the antibody Fc domain.

Embodiment P4: The protein according to embodiment P2 or P3, wherein the heavy chain variable domain of the scFv forms a disulfide bridge with the light chain variable domain of the scFv.

Embodiment P5: The protein according to embodiment P4, wherein the disulfide bridge is formed between C44 from the heavy chain variable domain and C100 from the light chain variable domain.

Embodiment P6: The protein according to embodiment P5, wherein the scFv is linked to the antibody Fc domain, wherein the light chain variable domain of the scFv is positioned at the N-terminus of the heavy chain variable domain of the scFv, and is linked to the heavy chain variable domain of the scFv via a flexible linker (GlyGlyGlyGlySer) 4 ((G4S)₄) (SEQ ID NO:143), and the Fab is linked to the antibody Fc domain.

Embodiment P7: The protein according to any one of embodiments P2-P6, wherein the heavy chain variable domain of the scFv is linked to the light chain variable domain of the scFv via a flexible linker.

Embodiment P8: The protein according to embodiment P7, wherein the flexible linker comprises (GlyGlyGlyGlySer) 4 ((G4S)₄) (SEQ ID NO:143).

Embodiment P9: The protein according to any one of embodiments P2-P8, wherein the heavy chain variable domain of the scFv is positioned at the N-terminus or the C-terminus of the light chain variable domain of the scFv.

Embodiment P10: The protein according to embodiment P9, wherein the light chain variable domain of the scFv is positioned at the N-terminus of the heavy chain variable domain of the scFv.

Embodiment P11: The protein according to any one of embodiments P1 to P10, wherein the Fab fragment is linked to the antibody Fc domain or a portion thereof sufficient to bind CD16 or the third antigen-binding site that binds CD16.

Embodiment P12: The protein according to embodiment P11, wherein the heavy chain portion of the Fab fragment comprises a heavy chain variable domain and a CH1 domain, and wherein the heavy chain variable domain is linked to the CH1 domain.

Embodiment P13: The protein according to embodiment P11 or P12, wherein the Fab is linked to the antibody Fc domain.

Embodiment P14: A protein according to any of the preceding embodiments comprising a sequence of SEQ ID NO:139.

Embodiment P15: A protein according to any one of embodiments P2-P14 comprising an scFv linked to an antibody Fc domain, wherein the scFv linked to the antibody Fc domain is represented by a sequence selected from SEQ ID NO:140 and SEQ ID NO:146.

Embodiment P16: A protein according to any of the preceding embodiments comprising a sequence of SEQ ID NO:141, SEQ ID NO:145, SEQ ID NO:147, or SEQ ID NO: 148.

Embodiment P17: A protein comprising a sequence at least 90% identical to an amino acid sequence of SEQ ID NO:139.

Embodiment P18: A protein comprising a sequence at least 95% identical to an amino acid sequence of SEQ ID NO:139.

Embodiment P19: A protein comprising a sequence at least 99% identical to an amino acid sequence of SEQ ID NO:139.

Embodiment P20: A protein comprising a sequence at least 90% identical to an amino acid sequence selected from SEQ ID NO:140 and SEQ ID NO:146.

Embodiment P21: A protein comprising a sequence at least 95% identical to an amino acid sequence selected from SEQ ID NO:140 and SEQ ID NO:146.

Embodiment P22: A protein comprising a sequence at least 99% identical to an amino acid sequence selected from SEQ ID NO:140 and SEQ ID NO:146.

Embodiment P23: The protein according any one of embodiments P1-P13, wherein the first antigen-binding site that binds NKG2D comprises a heavy chain variable domain at least 90% identical to an amino acid sequence selected from SEQ ID NO:86 and SEQ ID NO:94.

Embodiment P24: The protein according any one of embodiments P1-P13, wherein the first antigen-binding site that binds NKG2D comprises a heavy chain variable domain at least 90% identical to SEQ ID NO:86 and a light chain variable domain at least 90% identical to SEQ ID NO:90.

Embodiment P25: The protein according any one of embodiments P1-P13, wherein the first antigen-binding site that binds NKG2D comprises a heavy chain variable domain at least 95% identical to SEQ ID NO:86 and a light chain variable domain at least 95% identical to SEQ ID NO:90.

Embodiment P26: The protein according any one of embodiments P1-P13, wherein the first antigen-binding site that binds NKG2D comprises a heavy chain variable domain at least 90% identical to SEQ ID NO:94 and a light chain variable domain at least 90% identical to SEQ ID NO:98.

Embodiment P27: The protein according any one of embodiments P1-P13, wherein the first antigen-binding site that binds NKG2D comprises a heavy chain variable domain at least 95% identical to SEQ ID NO:94 and a light chain variable domain at least 95% identical to SEQ ID NO:98.

Embodiment P28: The protein according any one of embodiments P1-P13, wherein the first antigen-binding site that binds NKG2D comprises a heavy chain variable domain at least 90% identical to SEQ ID NO:144 and a light chain variable domain at least 90% identical to SEQ ID NO:98.

Embodiment P29: The protein according any one of embodiments P1-P13, wherein the first antigen-binding site that binds NKG2D comprises a heavy chain variable domain at least 95% identical to SEQ ID NO:144 and a light chain variable domain at least 95% identical to SEQ ID NO:98.

Embodiment P30: The protein according any one of embodiments P1-P13, wherein the first antigen-binding site that binds NKG2D comprises a heavy chain variable domain identical to SEQ ID NO:86 and a light chain variable domain identical to SEQ ID NO: 90.

Embodiment P31: The protein according any one of embodiments P1-P13, wherein the first antigen-binding site that binds NKG2D comprises a heavy chain variable domain identical to SEQ ID NO:94 and a light chain variable domain identical to SEQ ID NO: 98.

Embodiment P32: The protein according any one of embodiments P1-P13, wherein the first antigen-binding site that binds NKG2D comprises a heavy chain variable domain identical to SEQ ID NO:144 and a light chain variable domain identical to SEQ ID NO: 98.

Embodiment P33: The protein according any one of embodiments P1-P13 and P23-P32, wherein the antibody Fc domain comprises hinge and CH2 domains of a human IgG1 antibody.

Embodiment P34: The protein according to embodiment P33, wherein the Fc domain comprises an amino acid sequence at least 90% identical to amino acids 234-332 of a human IgG1 antibody.

Embodiment P35: The protein according to embodiment P33 or P34, wherein the Fc domain comprises amino acid sequence at least 90% identical to the Fc domain of human IgG1 and differs at one or more positions selected from the group consisting of Q347, Y349, T350, L351, S354, E356, E357, K360, Q362, S364, T366, L368, K370, N390, K392, T394, D399, S400, D401, F405, Y407, K409, T411, K439.

Embodiment P36: The protein according to any one of embodiments P1-P13 and P23-P34, wherein the Fc domain is an Fc domain of a human IgG1 comprising Q347R, D399V, and F405T substitutions.

Embodiment P37: The protein according to embodiment P36, wherein the Fc domain comprising the substitutions is linked to the scFv.

Embodiment P38: The protein according to any one of embodiments P1-P13 and P23-P34, wherein the Fc domain is an Fc domain of a human IgG1 comprising K360E and K409W substitutions.

Embodiment P39: The protein according to embodiment P38, wherein the Fc domain comprising the substitutions is linked to the Fab fragment.

Embodiment P40: The protein according to any one of embodiments P1-P13 and P23-P34, wherein the Fc domain is an Fc domain of a human IgG1 comprising a T366W substitution.

Embodiment P41: The protein according to embodiment P40, wherein the Fc domain comprising the substitution is linked to the Fab fragment.

Embodiment P42: The protein according to any one of embodiments P1-P13 and P23-P34, wherein the Fc domain is an Fc domain of a human IgG1 comprising T366S, L368A, and Y407V substitutions.

Embodiment P43: The protein according to embodiment P42, wherein the Fc domain comprising the substitutions is linked to the scFv.

Embodiment P44: A protein according to any one of embodiments P1-P43, wherein the protein binds to NKG2D with an affinity of K_{D} of 10 nM or lower.

Embodiment P45: A formulation comprising a protein according to any one of the preceding embodiments and a pharmaceutically acceptable carrier.

Embodiment P46: A cell comprising one or more nucleic acids expressing a protein according to any one of embodiments P1-P44.

Embodiment P47: A method of directly and/or indirectly enhancing tumor cell death, the method comprising exposing a tumor and natural killer cells to a protein according to any one of embodiments P1-P44.

Embodiment P48: A method of treating cancer, wherein the method comprises administering a protein according to any one of embodiments P1-P44 or a formulation according to embodiment P45 to a patient.

Embodiment P49: The method of embodiment P48, wherein the cancer is selected from the group consisting of breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, lung squamous, mesothelioma, liver cancer, mesothelioma, sarcoma, and gall bladder cancer.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A protein comprising:
(a) a first antigen-binding site comprising an Fab fragment that binds NKG2D;
(b) a second antigen-binding site comprising a single-chain variable fragment (scFv) that binds HER2; and
(c) an antibody Fc domain or a portion thereof sufficient to bind CD16, or a third antigen-binding site that binds CD16.

2. The protein according to claim 1, wherein the scFv is linked to the antibody Fc domain or a portion thereof sufficient to bind CD16, or the third antigen-binding site that binds CD16, via a hinge comprising Ala-Ser.

3. The protein according to claim 2, wherein the scFv is linked to the antibody Fc domain.

4. The protein according to any one of claims 1-3, wherein the scFv comprises a heavy chain variable domain and a light chain variable domain.

5. The protein according to claim 4, wherein the heavy chain variable domain of the scFv forms a disulfide bridge with the light chain variable domain of the scFv.

6. The protein according to claim 5, wherein the disulfide bridge is formed between C44 of the heavy chain variable domain and C100 of the light chain variable domain.

7. The protein according to any one of claims 4-6, wherein the light chain variable domain of the scFv is linked to the heavy chain variable domain of the scFv via a flexible linker.

8. The protein according to claim 7, wherein the flexible linker comprises the amino acid sequence of SEQ ID NO:143.

9. The protein according to claim 8, wherein the flexible linker consists of the amino acid sequence of SEQ ID NO:143.

10. The protein according to any one of claims 4-9, wherein the light chain variable domain is positioned to the N-terminus or C-terminus to the heavy chain variable domain.

11. The protein according to claim 10, wherein the light chain variable domain is positioned to the N-terminus of the heavy chain variable domain.

12. The protein according to any one of claims 1-11, wherein the Fab fragment is linked to the antibody Fc domain or a portion thereof sufficient to bind CD16, or the third antigen-binding site that binds CD16.

13. A protein according to any one of claims 1-12, wherein the first antigen-binding site that binds NKG2D comprises:
(a) a heavy chain variable domain comprising complementarity-determining region 1 (CDR1), complementarity-determining region 2 (CDR2), and complementarity-determining region 3 (CDR3) sequences represented by the amino acid sequences of SEQ ID NOs: 168, 96, and 169, respectively; and a light chain variable domain comprising CDR1, CDR2, and CDR3 sequences represented by the amino acid sequences of SEQ ID NOs: 99, 100, and 101, respectively;
(b) a heavy chain variable domain comprising CDR1, CDR2, and CDR3 sequences represented by the amino acid sequences of SEQ ID NOs: 168, 96, and 173, respectively; and a light chain variable domain comprising CDR1, CDR2, and CDR3 sequences represented by the amino acid sequences of SEQ ID NOs: 99, 100, and 101, respectively;
(c) a heavy chain variable domain comprising CDR1, CDR2, and CDR3 sequences represented by the amino acid sequences of SEQ ID NOs: 95, 96, and 97, respectively; and a light chain variable domain comprising CDR1, CDR2, and CDR3 sequences represented by the amino acid sequences of SEQ ID NOs: 99, 100, and 101, respectively;
(d) a heavy chain variable domain comprising CDR1, CDR2, and CDR3 sequences represented by the amino acid sequences of SEQ ID NOs: 166, 88, and 167, respectively; and
a light chain variable domain comprising CDR1, CDR2, and CDR3 sequences represented by the amino acid sequences of SEQ ID NOs: 91, 92, and 93, respectively;
(e) a heavy chain variable domain comprising CDR1, CDR2, and CDR3 sequences represented by the amino acid sequences of SEQ ID NOs: 162, 72, and 170, respectively; and a light chain variable domain comprising CDR1, CDR2, and CDR3 sequences represented by the amino acid sequences of SEQ ID NOs: 107, 108, and 109, respectively;
(f) a heavy chain variable domain comprising CDR1, CDR2, and CDR3 sequences represented by the amino acid sequences of SEQ ID NOs: 162, 72, and 163, respectively; and a light chain variable domain comprising CDR1, CDR2, and CDR3 sequences represented by the amino acid sequences of SEQ ID NOs: 75, 76, and 77, respectively;
(g) a heavy chain variable domain comprising CDR1, CDR2, and CDR3 sequences represented by the amino acid sequences of SEQ ID NOs: 164, 80, and 165, respectively; and a light chain variable domain comprising CDR1, CDR2, and CDR3 sequences represented by the amino acid sequences of SEQ ID NOs: 75, 76, and 85, respectively;
(h) a heavy chain variable domain comprising CDR1, CDR2, and CDR3 sequences represented by the amino acid sequences of SEQ ID NOs: 168, 96, and 176, respectively; and a light chain variable domain comprising CDR1, CDR2, and CDR3 sequences represented by the amino acid sequences of SEQ ID NOs: 99, 100, and 101, respectively;
(i) a heavy chain variable domain comprising CDR1, CDR2, and CDR3 sequences represented by the amino acid sequences of SEQ ID NOs: 168, 96, and 179, respectively; and a light chain variable domain comprising CDR1, CDR2, and CDR3 sequences represented by the amino acid sequences of SEQ ID NOs: 99, 100, and 101, respectively;
(j) a heavy chain variable domain comprising CDR1, CDR2, and CDR3 sequences represented by the amino acid sequences of SEQ ID NOs: 168, 96, and 182, respectively; and a light chain variable domain comprising CDR1, CDR2, and CDR3 sequences represented by the amino acid sequences of SEQ ID NOs: 99, 100, and 101, respectively;
(k) a heavy chain variable domain comprising CDR1, CDR2, and CDR3 sequences represented by the amino acid sequences of SEQ ID NOs: 168, 96, and 185, respectively; and a light chain variable domain comprising CDR1, CDR2, and CDR3 sequences represented by the amino acid sequences of SEQ ID NOs: 99, 100, and 101, respectively; or
(l) a heavy chain variable domain comprising CDR1, CDR2, and CDR3 sequences represented by the amino acid sequences of SEQ ID NOs: 168, 96, and 188, respectively; and a light chain variable domain comprising CDR1, CDR2, and CDR3 sequences represented by the amino acid sequences of SEQ ID NOs: 99, 100, and 101, respectively.

14. The protein according to any one of claims 1-13, wherein the first antigen-binding site that binds NKG2D comprises a heavy chain variable domain at least 90% identical to an amino acid sequence selected from SEQ ID NO:86 and SEQ ID NO:94.

15. The protein according to any one of claims 1-14, wherein the first antigen-binding site that binds NKG2D comprises:
(a) a heavy chain variable domain at least 90% identical to SEQ ID NO:94 and a light chain variable domain at least 90% identical to SEQ ID NO:98;
(b) a heavy chain variable domain at least 90% identical to SEQ ID NO:144 and a light chain variable domain at least 90% identical to SEQ ID NO:98; or
(c) a heavy chain variable domain at least 90% identical to SEQ ID NO:86 and a light chain variable domain at least 90% identical to SEQ ID NO:90.

16. The protein according to any one of claims 1-15, wherein the first antigen-binding site that binds NKG2D comprises:
(a) a heavy chain variable domain at least 95% identical to SEQ ID NO:94 and a light chain variable domain at least 95% identical to SEQ ID NO:98;
(b) a heavy chain variable domain at least 95% identical to SEQ ID NO:144 and a light chain variable domain at least 95% identical to SEQ ID NO:98; or
(c) a heavy chain variable domain at least 95% identical to SEQ ID NO:86 and a light chain variable domain at least 95% identical to SEQ ID NO:90.

17. The protein according to any one of claims 1-16, wherein the first antigen-binding site that binds NKG2D comprises:
(a) a heavy chain variable domain identical to SEQ ID NO:94 and a light chain variable domain identical to SEQ ID NO:98;
(b) a heavy chain variable domain identical to SEQ ID NO:144 and a light chain variable domain identical to SEQ ID NO:98; or
(c) a heavy chain variable domain identical to SEQ ID NO:86 and a light chain variable domain identical to SEQ ID NO:90.

18. A protein according to any one of claims 1-17, wherein the protein binds to NKG2D with a K_{D} of 2 nM to 120 nM, as measured by surface plasmon resonance.

19. The protein according to any one of claims 1-18, wherein the second antigen-binding site that binds HER2 comprises:
(a) a heavy chain variable domain comprising CDR1, CDR2, and CDR3 sequences represented by the amino acid sequences of SEQ ID NOs: 115, 116, and 117, respectively, and a light chain variable domain comprising CDR1, CDR2, and CDR3 sequences represented by the amino acid sequences of SEQ ID NOs: 119, 120, and 121, respectively;
(b) a heavy chain variable domain comprising CDR1, CDR2, and CDR3 sequences represented by the amino acid sequences of SEQ ID NOs: 123, 124, and 125, respectively, and a light chain variable domain comprising CDR1, CDR2, and CDR3 sequences represented by the amino acid sequences of SEQ ID NOs: 127, 128, and 129, respectively; or
(c) a heavy chain variable domain comprising CDR1, CDR2, and CDR3 sequences represented by the amino acid sequences of SEQ ID NOs: 131, 132, and 133, respectively, and a light chain variable domain comprising CDR1, CDR2, and CDR3 sequences represented by the amino acid sequences of SEQ ID NOs: 135, 136, and 137, respectively.

20. The protein according to any one of claims 1-19, wherein the second antigen-binding site that binds HER2 comprises:
(a) a heavy chain variable domain comprising an amino acid sequence at least 90% identical to SEQ ID NO:195 and a light chain variable domain comprising an amino acid sequence at least 90% identical to SEQ ID NO:196;
(b) a heavy chain variable domain comprising an amino acid sequence at least 90% identical to SEQ ID NO:197 and a light chain variable domain comprising an amino acid sequence at least 90% identical to SEQ ID NO:198; or
(c) a heavy chain variable domain comprising an amino acid sequence at least 90% identical to SEQ ID NO:199 and a light chain variable domain comprising an amino acid sequence at least 90% identical to SEQ ID NO:200.

21. The protein according to any one of claims 1-20, wherein the second antigen-binding site that binds HER2 comprises:
(a) a heavy chain variable domain comprising an amino acid sequence at least 95% identical to SEQ ID NO:195 and a light chain variable domain comprising an amino acid sequence at least 95% identical to SEQ ID NO:196;
(b) a heavy chain variable domain comprising an amino acid sequence at least 95% identical to SEQ ID NO:197 and a light chain variable domain comprising an amino acid sequence at least 95% identical to SEQ ID NO:198; or
(c) a heavy chain variable domain comprising an amino acid sequence at least 95% identical to SEQ ID NO:199 and a light chain variable domain comprising an amino acid sequence at least 95% identical to SEQ ID NO:200.

22. The protein according to any one of claims 1-21, wherein the second antigen-binding site that binds HER2 comprises:
(a) a heavy chain variable domain comprising an amino acid sequence identical to SEQ ID NO:195 and a light chain variable domain comprising an amino acid sequence identical to SEQ ID NO:196;
(b) a heavy chain variable domain comprising an amino acid sequence identical to SEQ ID NO:197 and a light chain variable domain comprising an amino acid sequence identical to SEQ ID NO:198; or
(c) a heavy chain variable domain comprising an amino acid sequence identical to SEQ ID NO:199 and a light chain variable domain comprising an amino acid sequence identical to SEQ ID NO:200.

23. The protein according to any one of claims 1-22, wherein the second antigen-binding site that binds HER2 comprises the amino acid sequence of SEQ ID NO:139, SEQ ID NO: 189, or SEQ ID NO:171.

24. The protein according to claim 23, wherein the second antigen-binding site that binds HER2 comprises the amino acid sequence of SEQ ID NO:139.

25. The protein according to any one of claims 1-24, wherein the antibody Fc domain comprises hinge and CH2 domains of a human IgG1 antibody.

26. The protein according to claim 25, wherein the antibody Fc domain comprises an amino acid sequence at least 90% identical to amino acids 234-332 of a human IgG1 antibody.

27. The protein according to claim 25 or 26, wherein the antibody Fc domain comprises amino acid sequence at least 90% identical to the Fc domain of human IgG1 and differs at one or more positions selected from the group consisting of Q347, Y349, T350, L351, S354, E356, E357, K360, Q362, S364, T366, L368, K370, N390, K392, T394, D399, S400, D401, F405, Y407, K409, T411, K439.

28. The protein according to any one of claims 1-27, wherein the antibody Fc domain is an Fc domain of a human IgG1 comprising Q347R, D399V, and F405T substitutions.

29. The protein according to claim 28, wherein the antibody Fc domain comprising the substitutions is linked to the scFv.

30. The protein according to any one of claims 1-29, wherein the antibody Fc domain is an Fc domain of a human IgG1 comprising K360E and K409W substitutions.

31. The protein according to claim 30, wherein the antibody Fc domain comprising the substitutions is linked to the Fab fragment.

32. The protein according to any one of claims 1-27, wherein the antibody Fc domain is an Fc domain of a human IgG1 comprising a T366W substitution.

33. The protein according to claim 32, wherein the antibody Fc domain comprising the substitution is linked to the Fab fragment.

34. The protein according to any one of claims 1-27 and 32-33, wherein the antibody Fc domain is an Fc domain of a human IgG1 comprising T366S, L368A, and Y407V substitutions.

35. The protein according to claim 34, wherein the antibody Fc domain comprising the substitutions is linked to the scFv.

36. The protein according to any of claims 1-35, comprising a sequence of SEQ ID NO: 141, SEQ ID NO:145, SEQ ID NO:147, SEQ ID NO:194, SEQ ID NO:155, or SEQ ID NO: 148.

37. The protein according to any one of claims 1-36, wherein the scFv linked to the antibody Fc domain is represented by the amino acid sequence of SEQ ID NO:140 or SEQ ID NO: 146.

38. A protein comprising:
(a) a first polypeptide comprising an amino acid sequence at least 90% identical to SEQ ID NO:141;
(b) a second polypeptide comprising an amino acid sequence at least 90% identical to SEQ ID NO:140, SEQ ID NO: 190, or SEQ ID NO:192; and
(c) a third polypeptide comprising an amino acid sequence at least 90% identical to SEQ ID NO:142.

39. The protein according to claim 38, wherein the second polypeptide comprising an amino acid sequence at least 90% identical to SEQ ID NO:140.

40. A protein comprising:
(a) a first polypeptide comprising an amino acid sequence at least 95% identical to SEQ ID NO:141;
(b) a second polypeptide comprising an amino acid sequence at least 95% identical to SEQ ID NO:140, SEQ ID NO: 190, or SEQ ID NO: 192; and
(c) a third polypeptide comprising an amino acid sequence at least 95% identical to SEQ ID NO:142.

41. The protein according to claim 40, wherein the second polypeptide comprising an amino acid sequence at least 95% identical to SEQ ID NO:140.

42. A protein comprising:
(a) a first polypeptide comprising the amino acid sequence of SEQ ID NO:141;
(b) a second polypeptide comprising the amino acid sequence of SEQ ID NO:140, SEQ ID NO: 190, or SEQ ID NO: 192; and
(c) a third polypeptide comprising the amino acid sequence of SEQ ID NO:142.

43. The protein according to claim 42, wherein the second polypeptide comprises the amino acid sequence of SEQ ID NO:140.

44. A formulation comprising a protein according to any one of the preceding claims and a pharmaceutically acceptable carrier.

45. A cell comprising one or more nucleic acids expressing a protein according to any one of claims 1-43.

46. A method of directly and/or indirectly enhancing tumor cell death, the method comprising exposing a tumor and natural killer cells to a protein according to any one of claims 1-43.

47. A method of treating cancer, wherein the method comprises administering a protein according to any one of claims 1-43 or a formulation according to claim 44 to a patient.

48. The method of claim 47, wherein the cancer is selected from the group consisting of breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, lung squamous, mesothelioma, liver cancer, mesothelioma, sarcoma, and gall bladder cancer.
